SEQUENCE LISTING (78 sequences)

SEQ ID NO: 1 - DNA, 720 bp, Homo sapiens
atgggtgacctgtggttattcctgctcctgcccctctcagccttccatggagtcaaaggctgtttagaatgtgaccccaaatttatagaggatgttggctccttgctgggaaatctgataccttcagaagtccccggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctctatttctcatattgctggcaacagctgtaatactgggaagtgctgtgttactattccatttttgcatctttcatcggaggaaaatgaaggcaatacgaaggtcgctaaaggaatatgtggagaagaaacttgaagaattaatggggaagatagatgagaaggaggagaaagactttagactcagaaaataa

SEQ ID NO: 2 - protein, 239 aa, Homo sapiens
MGDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIALFLILLATAVILGSAVLLFHFCIFHRRKMKAIRRSLKEYVEKKLEELMGKIDEKEEKDFRLRK

SEQ ID NO: 3 - DNA, 660 bp, Homo sapiens
tgtttagaatgtgaccccaaatttatagaggatgttggctccttgctgggaaatctgataccttcagaagtccccggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctctatttctcatattgctggcaacagctgtaatactgggaagtgctgtgttactattccatttttgcatctttcatcggaggaaaatgaaggcaatacgaaggtcgctaaaggaatatgtggagaagaaacttgaagaattaatggggaagatagatgagaaggaggagaaagactttagactcagaaaataa

SEQ ID NO: 4 - protein, 219 aa, Homo sapiens
CLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIALFLILLATAVILGSAVLLFHFCIFHRRKMKAIRRSLKEYVEKKLEELMGKIDEKEEKDFRLRK

SEQ ID NO: 5 - DNA, 531 bp, Homo sapiens
atgggtgacctgtggttattcctgctcctgcccctctcagccttccatggagtcaaaggctgtttagaatgtgaccccaaatttatagaggatgttggctccttgctgggaaatctgataccttcagaagtccccggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctcta

SEQ ID NO: 6 - protein, 177 aa, Homo sapiens
MGDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIAL

SEQ ID NO: 7 - DNA, 471 bp, Homo sapiens
tgtttagaatgtgaccccaaatttatagaggatgttggctccttgctgggaaatctgataccttcagaagtccccggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctcta

SEQ ID NO: 8 - protein, 157 aa, Homo sapiens
CLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIAL

SEQ ID NO: 9 - DNA, 117 bp, Homo sapiens
catcggaggaaaatgaaggcaatacgaaggtcgctaaaggaatatgtggagaagaaacttgaagaattaatggggaagatagatgagaaggaggagaaagactttagactcagaaaa

SEQ ID NO: 10 - protein, 39 aa, Homo sapiens
HRRKMKAIRRSLKEYVEKKLEELMGKIDEKEEKDFRLRK

SEQ ID NO: 11 - DNA, 1023 bp, Homo sapiens
atgcatgtggcagaggtagcagtgaacgtgatattgctcctaagcatggggtggacatcagactctctctgctcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcctggaaccagtgccattttgtacaacataacagacggtatagatccggatttgctggtttttgaacaatctcccacatatctaaatactcgttcttcatccaatagatgggacagactaaggattctaaaagctatgaatttagataaacaaaccaccacgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacctggactcaacagtagcaaacaattactaaacaaaaccaaaggatacaatagcagaaaccacacatctgcaaatgaagatgaggtatctgtgacttcaaagacttggctggtttctgtggccctttgcacctctgtcatctttctcggctgttgtatagtcatcctggcatctggatgctgtggaaagcagcagggccagtataaaccaggacagagaaaatcaggatccttgcaaattaaaaaccgtaaccatatgaaggagaactcttcatag

SEQ ID NO: 12 - protein, 340 aa, Homo sapiens
MHVAEVAVNVILLLSMGWTSDSLCSPTIFYRDCWIRRFPGLLINLEESQKLGAQFLKYYSESTGQKCSRSCCLRKDVSCNLAVFYHSPIHDNINCLHVHCPTLESCILEPGTSAILYNITDGIDPDLLVFEQSPTYLNTRSSSNRWDRLRILKAMNLDKQTTTINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTWLVSVALCTSVIFLGCCIVILASGCCGKQQGQYKPGQRKSGSLQIKNRNHMKENSS

SEQ ID NO: 13 - DNA, 951 bp, Homo sapiens
tcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcctggaaccagtgccattttgtacaacataacagacggtatagatccggatttgctggtttttgaacaatctcccacatatctaaatactcgttcttcatccaatagatgggacagactaaggattctaaaagctatgaatttagataaacaaaccaccacgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacctggactcaacagtagcaaacaattactaaacaaaaccaaaggatacaatagcagaaaccacacatctgcaaatgaagatgaggtatctgtgacttcaaagacttggctggtttctgtggccctttgcacctctgtcatctttctcggctgttgtatagtcatcctggcatctggatgctgtggaaagcagcagggccagtataaaccaggacagagaaaatcaggatccttgcaaattaaaaaccgtaaccatatgaaggagaactcttcatag

SEQ ID NO: 14 - protein, 316 aa, Homo sapiens
SPTIFYRDCWIRRFPGLLINLEESQKLGAQFLKYYSESTGQKCSRSCCLRKDVSCNLAVFYHSPIHDNINCLHVHCPTLESCILEPGTSAILYNITDGIDPDLLVFEQSPTYLNTRSSSNRWDRLRILKAMNLDKQTTTINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTWLVSVALCTSVIFLGCCIVILASGCCGKQQGQYKPGQRKSGSLQIKNRNHMKENSS

SEQ ID NO: 15 - DNA, 855 bp, Homo sapiens
atgcatgtggcagaggtagcagtgaacgtgatattgctcctaagcatggggtggacatcagactctctctgctcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcctggaaccagtgccattttgtacaacataacagacggtatagatccggatttgctggtttttgaacaatctcccacatatctaaatactcgttcttcatccaatagatgggacagactaaggattctaaaagctatgaatttagataaacaaaccaccacgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacctggactcaacagtagcaaacaattactaaacaaaaccaaaggatacaatagcagaaaccacacatctgcaaatgaagatgaggtatctgtgacttcaaagacttgg

SEQ ID NO: 16 - protein, 285 aa, Homo sapiens
MHVAEVAVNVILLLSMGWTSDSLCSPTIFYRDCWIRRFPGLLINLEESQKLGAQFLKYYSESTGQKCSRSCCLRKDVSCNLAVFYHSPIHDNINCLHVHCPTLESCILEPGTSAILYNITDGIDPDLLVFEQSPTYLNTRSSSNRWDRLRILKAMNLDKQTTTINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTW

SEQ ID NO: 17 - DNA, 783 bp, Homo sapiens
tcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcctggaaccagtgccattttgtacaacataacagacggtatagatccggatttgctggtttttgaacaatctcccacatatctaaatactcgttcttcatccaatagatgggacagactaaggattctaaaagctatgaatttagataaacaaaccaccacgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacctggactcaacagtagcaaacaattactaaacaaaaccaaaggatacaatagcagaaaccacacatctgcaaatgaagatgaggtatctgtgacttcaaagacttgg

SEQ ID NO: 18 - protein, 261 aa, Homo sapiens
SPTIFYRDCWIRRFPGLLINLEESQKLGAQFLKYYSESTGQKCSRSCCLRKDVSCNLAVFYHSPIHDNINCLHVHCPTLESCILEPGTSAILYNITDGIDPDLLVFEQSPTYLNTRSSSNRWDRLRILKAMNLDKQTTTINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTW

SEQ ID NO: 19 - DNA, 246 bp, Homo sapiens
ttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcct

SEQ ID NO: 20 - protein, 82 aa, Homo sapiens
FYRDCWIRRFPGLLINLEESQKLGAQFLKYYSESTGQKCSRSCCLRKDVSCNLAVFYHSPIHDNINCLHVHCPTLESCILEP

SEQ ID NO: 21 - DNA, 96 bp, Homo sapiens
tgctgtggaaagcagcagggccagtataaaccaggacagagaaaatcaggatccttgcaaattaaaaaccgtaaccatatgaaggagaactcttca

SEQ ID NO: 22 - protein, 32 aa, Homo sapiens
CCGKQQGQYKPGQRKSGSLQIKNRNHMKENSS

SEQ ID NO: 23 - DNA, 638 bp, Homo sapiens
atgggtgacctgtggttattcctgctcctgcccctctcagccttccatggagtcaaaggctgtttagaatgtgaccccaaatttatagaggatgttggctccttgctgggaaatctgataccttcagaagtccctggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctctatttctcatattgctggcaacagctgtaatactgggaagtgctgtgttactattccatttttgcatctttcatcggaggaaaatgaaggcaatacgaaggtcgctaaa

SEQ ID NO: 24 - protein, 212 aa, Homo sapiens
MGDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIALFLILLATAVILGSAVLLFHFCIFHRRKMKAIRRSL

SEQ ID NO: 25 - DNA, 559 bp, Homo sapiens
ggtgacctgtggttattcctgctcctgcccctctcagccttccatggagtcaaaggctgtttagaatgtgaccccaaatttatagaggatgttggctccttgctgggaaatctgataccttcagaagtccctggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgttgtggttgaaggtcccattcttgattgttggacgtgtcttcgcatgactaacaggtgcttcaaaggagaatattgtggagacgaggatccaagaaaggctgagaatcgagagattgctctatttctcatattgctggcaacagctgtaatactgggaagtgctgtgttac

SEQ ID NO: 26 - protein, 186 aa, Homo sapiens
GDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIVVVEGPILDCWTCLRMTNRCFKGEYCGDEDPRKAENREIALFLILLATAVILGSAVL

SEQ ID NO: 27 - DNA, 578 bp, Homo sapiens
ggtgacctgtggttattcctgctcctgcccctctcagccttccatggagtcaaaggctgtttagaatgtgaccccaaatttatagaggatgttggctccttgctgggaaatctgataccttcagaagtccctggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctcagaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctctatttctcatattgctggcaacagctgtaatactggggaagtgctgtgttac

SEQ ID NO: 28 - protein, 192 aa, Homo sapiens
GDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIACSEDCIVVEGPILDCWTCLRMTNRCFKGEYCGDEDPRKAENREIALFLILLATAVILGKCCV

SEQ ID NO: 29 - DNA, 543 bp, Homo sapiens
ggtgacctgtggttattcctgctcctgcccctctcagccttccatggagtcaaaggctgtttagaatgtgaccccaaatttatagaggatgttggctccttgctgggaaatctgataccttcagaagtccctggccgaactcagctgcttgaacggcagattaaggagatgattcatttaagcttcaagctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgcttgttctgaagattgcattgtggttgaaggtcccattcttgattgttggacgtgtcttcgcatgactaacaggtgcttcaaaggagaatattgtggagacgaggatccaagaaaggctgagaatcgagagattgctctatttctcatattgccggcaacagctgtaatactgggaagtgctgtgttac

SEQ ID NO: 30 - protein, 70 aa, Homo sapiens
GDLWLFLLLPLSAFHGVKGCLECDPKFIEDVGSLLGNLIPSEVPGRTQLLERQIKEMIHLSFKLFSRLLS

SEQ ID NO: 31 - DNA, 510 bp, Homo sapiens
ggtgacctgtggttattcctgctcctgcccctctcagccttccatggagtcaaaggctgtttagaatgtgaccccaaatttatagaggatgttggctccttgctgggaaatctgataccttcagaagtccctggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgttgtggttgaaggtcccattcttgattgttggacgtgtcttcgcatgactaacaggtgcttcaaaggagaatattgtggagacgaggatccaagaaaggctgagaatcgagagattgctcta

SEQ ID NO: 32 - protein, 170 aa, Homo sapiens
GDLWLFLLLPLSAFHGVKGCLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIVVVEGPILDCWTCLRMTNRCFKGEYCGDEDPRKAENREIAL

SEQ ID NO: 33 - DNA, 792 bp, Homo sapiens
atgcattcggcagaggtagcagtgaacgtgatattgctcctaagcatggggtggacatcagactctctctgctcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatggtatagatccggatttgctggtttttgaacaatctcccacatatctaaatactcgttcttcatccaatagatgggacagactaaggattctaaaagctatgaattcagataaacaaaccaccatgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacccggactcaacagtagcaaacaattactaaacaaaaccaaaggatacaatagcagaaaccacacatctgcaaatgaagatgaggtatctgtgacttcaaagacttggctggtttctgtggccctttgcacctctgtcatctttctcggctgttgtatagtcatcctggcagatggatgc

SEQ ID NO: 34 - protein, 264 aa, Homo sapiens
MHSAEVAVNVILLLSMGWTSDSLCSPTIFYRDCWIRRFPGLLINLEESQKLGAQFLKYYSESTGQKCSRSCCLRKDGIDPDLLVFEQSPTYLNTRSSSNRWDRLRILKAMNSDKQTTMINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTWLVSVALCTSVIFLGCCIVILADGC

SEQ ID NO: 35 - DNA, 720 bp, Homo sapiens
atgcattcggcagaggtagcagtgaacgtgatattgctcctaagcatggggtggacatcagactctctctgctcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatggtatagatccggatttgctggtttttgaacaatctcccacatatctaaatactcgttcttcatccaatagatgggacagactaaggattctaaaagctatgaattcagataaacaaaccaccatgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacccggactcaacagtagcaaacaattactaaacaaaaccaaaggatacaatagcagaaaccacacatctgcaaatgaagatgaggtatctgtgacttcaaagacttgg

SEQ ID NO: 36 - protein, 241 aa, Homo sapiens
MHSAEVAVNVILLLSMGWTSDSLCSPTIFYRDCWIRRFPGLLINLEESQKLGAQFLKYYSESTGQKCSRSCCLRKDGIDPDLLVFEQSPTYLNTRSSSNRWDRLRILKAMNSDKQTTMINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTWI

SEQ ID NO: 37 - DNA, 648 bp, Homo sapiens
tcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatggtatagatccggatttgctggtttttgaacaatctcccacatatctaaatactcgttcttcatccaatagatgggacagactaaggattctaaaagctatgaattcagataaacaaaccaccatgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacccggactcaacagtagcaaacaattactaaacaaaaccaaaggatacaatagcagaaaccacacatctgcaaatgaagatgaggtatctgtgacttcaaagacttgg

SEQ ID NO: 38 - protein, 216 aa, Homo sapiens
SPTIFYRDCWIRRFPGLLINLEESQKLGAQFLKYYSESTGQKCSRSCCLRKDGIDPDLLVFEQSPTYLNTRSSSNRWDRLRILKAMNSDKQTTMINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTW

SEQ ID NO: 39 - DNA, 453 bp, Homo sapiens
tgtttagaatgtgaccccaaatttatagaggatgttggctccttgctgggaaatctgataccttcagaagtccctggccgaactcagctgcttgaacggcagattaaggagatgattcatttaagcttcaaggtctcccacagtgacaagaggcttcgggtgttggctgttcagcaggttgttaagttgagaacatggttgaagaatgaattttataaactgggcaatgaaacatggaaaggtgtctttatctatcaaggcaagcttctcgatgtctgccaaaacctggaatccaaactgaaagaattactaaagaacttctctgaaattgttgtggttgaaggtcccattcttgattgttggacgtgtcttcgcatgactaacaggtgcttcaaaggagaatattgtggagacgaggatccaagaaaggctgagaatcgagagattgctcta

SEQ ID NO: 40 - protein, 151 aa, Homo sapiens
CLECDPKFIEDVGSLLGNLIPSEVPGRTQLLERQIKEMIHLSFKVSHSDKRLRVLAVQQVVKLRTWLKNEFYKLGNETWKGVFIYQGKLLDVCQNLESKLKELLKNFSEIVVVEGPILDCWTCLRMTNRCFKGEYCGDEDPRKAENREIAL

SEQ ID NO: 41 - DNA, 1046 bp, Homo sapiens
gcgaattcatgcatgtggcagaggtagcagtgaacgtgatattgctcctaagcatggggtggacatcagactctctctgctcacccacaattttttacagagactgctggatccgtcgcttcccaggtcttctaatcaatctggaggagtctcagaagctgggagcccagttcttgaagtattattctgaaagcactggccagaagtgcagtaggagctgctgtcttcggaaggatgtttcctgtaacctggctgtcttctaccacagtcctattcatgacaatatcaactgcctccatgttcactgcccaacactggagagctgcatattagagcctggaaccagtgccattttgtacaacataacagacggtatagatccggatttgctggtttttgaacaatctcccacatatctaaatactcgttcttcatccaatagatgggacagactaaggattctaaaagctatgaatttagataaacaaaccaccacgataaatggtatgctgccatccacagaggctccatcctcaaccacgcatcaagatttggttgtaaacacaaacagtaccagttattctaaggaattaaccacagatttttgggcaagatttacttccctgaatgagtccattaccacaaagataaataaggtgtcaccaagtactgatttcatcagcaatccagataataagactatttctcctttctttgaacccatagacacaaaactttctcatatgcctgttccacctggactcaacagtagcaaacaattactaaacaaaaccaaaggatacaatagcagaaaccacacatctgcaaatgaagatgaggtatctgtgacttcaaagacttggctggtttctgtggccctttgcacctctgtcatctttctcggctgttgtatagtcatcctggcatctggatgctgtggaaagcagcagggccagtataaaccaggacagagaaaatcaggatccttgcaaattaaaaaccgtaaccatatgaaggagaactcttcatagtaaaaacgaattcgc

SEQ ID NO: 42 - protein, 340 aa, Homo sapiens; identical to SEQ ID NO: 12

SEQ ID NO: 43 - protein, 81 aa, Homo sapiens
GEYCGDEDPRKAENREIALFLILLATAVILGSAVLLFHFCIFHRRKMKAIRRSLKEYVEKKLEELMGKIDEKEEKDFRLRK

SEQ ID NO: 44 - protein, 29 aa, Homo sapiens
DPRKAENREIALFLILLATAVILGSAVLL

SEQ ID NO: 45 - protein, 219 aa, Homo sapiens
GIDPDLLVFEQSPTYLNTRSSSNRWDRLRILKAMNLDKQTTTINGMLPSTEAPSSTTHQDLVVNTNSTSYSKELTTDFWARFTSLNESITTKINKVSPSTDFISNPDNKTISPFFEPIDTKLSHMPVPPGLNSSKQLLNKTKGYNSRNHTSANEDEVSVTSKTWLVSVALCTSVIFLGCCIVILASGCCGKQQGQYKPGQRKSGSLQIKNRNHMKENSS

SEQ ID NO: 46 - protein, 76 aa, Homo sapiens
MHVAEVAVNVILLLSMGWTSDSLCSPTIFYRDCWIRRFPGLLINLEESQKLGAQFLKYYSESTGQKCSRSCCLRKD

SEQ ID NO: 47 - DNA, 20 bp, Artificial Sequence
  PCR primer
atgggtgacctgtggttatt

SEQ ID NO: 48 - DNA, 20 bp, Artificial Sequence
  PCR primer
tttagcgaccttcgtattgc

SEQ ID NO: 49 - DNA, 18 bp, Artificial Sequence
  PCR primer
ggtgacctgtggttattc

SEQ ID NO: 50 - DNA, 21 bp, Artificial Sequence
  PCR primer
gtaacacagcacttcccagta

SEQ ID NO: 51 - DNA, 36 bp, Artificial Sequence
  PCR primer
gcaggcttcgccaccatgggtgacctgtggttattc

SEQ ID NO: 52 - DNA, 34 bp, Artificial Sequence
  PCR primer
gtgatggtgatggtgacttaacaacctgctgaac

SEQ ID NO: 53 - DNA, 37 bp, Artificial Sequence
  PCR primer
ggggacaagtttgtacaaaaaagcaggcttcgccacc

SEQ ID NO: 54 - DNA, 51 bp, Artificial Sequence
  PCR primer
ggggaccactttgtacaagaaagctgggtttcaatggtgatggtgatggtg

SEQ ID NO: 55 - DNA, 20 bp, Artificial Sequence
  PCR primer
agcctcagacagtggttcaa

SEQ ID NO: 56 - DNA, 20 bp, Artificial Sequence
  PCR primer
gatggaggtggacgtgtcag

SEQ ID NO: 57 - DNA, 24 bp, Artificial Sequence
  PCR primer
cgacgttgtaaaacgacggccagt

SEQ ID NO: 58 - DNA, 17 bp, PCR primer
caggaaacagctatgac

SEQ ID NO: 59 - DNA, 19 bp, Artificial Sequence
  PCR primer
taatacgactcactatagg

SEQ ID NO: 60 - DNA, 18 bp, Artificial Sequence
  PCR primer
attaaccctcactaaagg

SEQ ID NO: 61 - DNA, 20 bp, Artificial Sequence
  PCR primer
atgcatgtggcagaggtagc

SEQ ID NO: 62 - DNA, 20 bp, Artificial Sequence
  PCR primer
gcatccagatgccaggatga

SEQ ID NO: 63 - DNA, 52 bp, Artificial Sequence
  PCR primer
ctaaaagctatgaatttagataaacaaaccaccacgataaatggtatgctgc

SEQ ID NO: 64 - DNA, 21 bp, Artificial Sequence
  PCR primer
cttgaaagcatataatcttgc

SEQ ID NO: 65 - DNA, 28 bp, Artificial Sequence
  PCR primer
gcgaattcatgcatgtggcagaggtagc

SEQ ID NO: 66 - DNA, 28 bp, Artificial Sequence
  PCR primer
gcgaattcgtttttactatgaagagttc

SEQ ID NO: 67 - DNA, 52 bp, Artificial Sequence
  PCR primer
gcagcataccatttatcgtggtggtttgtttatctaaattcatagcttttag

SEQ ID NO: 68 - DNA, 37 bp, Artificial Sequence
  PCR primer
gcaggcttcgccaccatgcatgtggcagaggtagcag

SEQ ID NO: 69 - DNA, 31 bp, Artificial Sequence
  PCR primer
gtgatggtgatggtgccaagtctttgaagtc

SEQ ID NO: 70 - DNA, 20 bp, Artificial Sequence
  PCR primer
gctccttgctgggaaatctg

SEQ ID NO: 71 - DNA, 25 bp, Artificial Sequence
  PCR primer
tgttctcaacttaacaacctgctga

SEQ ID NO: 72 - DNA, 18 bp, Artificial Sequence
  PCR primer
ccacccatggcaaattcc

SEQ ID NO: 73 - DNA, 22 bp, Artificial Sequence
  PCR primer
gatgggatttccattgatgaca

SEQ ID NO: 74 - DNA, 21 bp, Artificial Sequence
  PCR primer
cctagtcccagggctttgatt

SEQ ID NO: 75 - DNA, 21 bp, Artificial Sequence
  PCR primer
ctgtgctcccactcctgattt

SEQ ID NO: 76 - DNA, 22 bp, Artificial Sequence
  PCR primer
tgcatattagagcctggaacca

SEQ ID NO: 77 - DNA, 27 bp, Artificial Sequence
  PCR primer
ccatctattggatgaagaacgagtatt

SEQ ID NO: 78 - protein, 13 aa, Artificial Sequence
  Fusion protein linker sequence
EFGAGLVLGGQFM

(19) United States
(12) Patent Application Publication
(10) Pub. No.: US 2009/0215672 A1
(43) Pub. Date: Aug. 27, 2009

(54) Cys-Rich, Cell Surface Glycoproteins

(75) Inventors: Christine Power, Thoiry (FR); David Michalovich, London (GB); Richard James Mitter, London (GB)

Correspondence Address: SALIWANCHIK LLOYD & SALIWANCHIK;A PROFESSIONAL ASSOCIATION, PO Box 142950, GAINESVILLE, FL, 32614, US

(73) Assignee: ARES TRADING S.A., Aubonne (CH)

(21) Appl. No.: 12/096,135

(22) PCT Filed: Dec. 7, 2006
(86) PCT No.: PCT/GB2006/004590

§ 371 Date: Mar. 26, 2009

(30) Foreign Application Priority Data
Dec. 7, 2005 (GB) 0524984.2

(51) Int. Cl. A61K 38/17; C07K 14/47; C12N 15/12; C12N 15/85; C12N 5/10; C07K 16/18; C12Q 1/68; A61K 31/711; A01K 67/027; A61B 10/00; G01N 33/566; A61P 35/00; A61P 35/02; A61P 15/08; A61P 11/00; A61P 19/02; A61P 19/10; A61P 37/00; A61P 17/06; A61P 11/06; A61P 9/10; A61P 9/12; A61P 25/28; A61P 29/00; A61P 3/04; A61P 25/00; A61P 31/04; A61P 31/18; A61P 31/10; A61P 33/00

(52) U.S. Cl. 514/8; 530/350; 536/23.5; 435/320.1; 435/325; 530/387.9; 435/6; 514/44 R; 800/13; 800/3; 435/7.1

(57) ABSTRACT

This invention relates to novel proteins, termed INSP206 and INSP208 herein identified as Cys-rich, cell surface glycoproteins, and to the use of these proteins and nucleic acid sequences from the encoding genes in the diagnosis, prevention and treatment of disease.

This invention relates to novel proteins, termed INSP206 and INSP208 herein identified as Cys-rich, cell surface glycoproteins, and to the use of these proteins and nucleic acid sequences from the encoding genes in the diagnosis, prevention and treatment of disease.

All publications, patents and patent applications cited herein are incorporated in full by reference.

### BACKGROUND

The process of drug discovery is presently undergoing a fundamental revolution as the era of functional genomics comes of age. The term “functional genomics” applies to an approach utilising bioinformatics tools to ascribe function to protein sequences of interest. Such tools are becoming increasingly necessary as the speed of generation of sequence data is rapidly outpacing the ability of research laboratories to assign functions to these protein sequences.

As bioinformatics tools increase in potency and in accuracy, these tools are rapidly replacing the conventional techniques of biochemical characterisation. Indeed, the advanced bioinformatics tools used in identifying the present invention are now capable of outputting results in which a high degree of confidence can be placed.

Various institutions and commercial organisations are examining sequence data as they become available and significant discoveries are being made on an on-going basis. However, there remains a continuing need to identify and characterise further genes and the polypeptides that they encode, as targets for research and for drug discovery.

### INTRODUCTION

### Secreted Proteins

The ability for cells to make and secrete extracellular proteins is central to many biological processes. Enzymes, growth factors, extracellular matrix proteins and signalling molecules are all secreted by cells. This is through fusion of a secretory vesicle with the plasma membrane. In most cases, but not all, proteins are directed to the endoplasmic reticulum and into secretory vesicles by a signal peptide. Signal peptides are cis-acting sequences that affect the transport of polypeptide chains from the cytoplasm to a membrane bound compartment such as a secretory vesicle. Polypeptides that are targeted to the secretory vesicles are either secreted into the extracellular matrix or are retained in the plasma membrane. The polypeptides that are retained in the plasma membrane will have one or more transmembrane domains. Examples of secreted proteins that play a central role in the functioning of a cell are cytokines, hormones, extracellular matrix proteins (adhesion molecules), proteases, and growth and differentiation factors.

### Cell Surface Glycoproteins and Receptors

The outer surface of the cell membrane plays a major role in the assembly and maintenance of tissue integrity. The outer surfaces of developing and differentiated cells contain receptor molecules that recognise systemic signals, ligands or hormones. The binding or dissociation of the ligands controls some of the differentiated functions of the cell, keeping it in tune with the needs of the whole system.

The outer surface is also coated with glycoproteins and proteoglycans. These large complexes of protein and polysaccharides provide a tissue-specific matrix within which cells of like function can operate together as a coherent tissue. In embryo-genesis the sorting out and tying together of cells with a common function is facilitated, and probably controlled, thorough the molecular specificities of the glycoprotein and proteoglycan surfaces of the cells.

The carbohydrate chains are N- and O-glycosidically-linked to glycoproteins forming complex structures. The N- and O-glycan chains are assembled in the endoplasmic reticulum and the Golgi by a controlled sequence of glycosyltransferase and glycosidase processing reactions subject to specific regulatory events (for example, at the level of gene expression or localisation of the enzyme). This complex regulation results in many hundreds of structures, the range of which varies amongst cell/tissue types or development/differentiation status.

There is a growing interest in diseases caused by defective glycosylation, and in therapeutic glycoproteins produced through recombinant DNA technology route.

At this scope, given that glycoproteins are typically expressed as mixtures of glycoforms, various technologies have been developed to obtain homogeneous glycopeptide and glycoprotein materials for experiments relevant to the biological investigation of glycoproteins (Grogan M J et al., Annu Rev Biochem. 2002; 71: 593-634).

Cell-surface receptors are important components of any prokaryotic or eukaryotic cell and have been shown to play important roles in a large number of diverse physiological functions, many of which are involved in disease processes. Alteration of their activity is a means to alter the relevant disease phenotype and as such identification of novel cell-surface receptors is highly desirable.

### MANSC Domain Containing Proteins

MANSC (motif at N terminus with seven cysteines) is a novel domain with a seven cysteine motif that is present at the N terminus of membrane and extracellular proteins, including lipoprotein receptor-related protein 11 (LRP-11), hepatocyte growth factor activator inhibitor 1 (HAI-1), polycystic kidney disease 1-like and a number of uncharacterised proteins. These domains were first discovered by Guo et al. (Trends Biochem. Sci. 2004 29(4):172-4) and described as containing seven conserved cysteine residues. These residues are likely to form functionally important disulphide bonds that determine the structure and function of the conserved domain.

HAI-1 has been shown to inhibit proteases involved in the proteolytic conversion and maturation of precursor proteins into molecules stimulating tumour cell-cell interactions, matrix adhesion, migration, invasion and angiogenesis (Kirchhofer D. et al. 2003 J Biol Chem, 278(38):36341-9 & Denda K et al. 2002 J Biol Chem, 277(16):14053-9). LRP-11 (also known as LR11 or sorLA) is synthesised as a proreceptor that is cleaved by furin in late Golgi compartments and its proteolytic activation can allow the binding of multiple ligands (Jacobsen L et al. 2001 J Biol Chem 276(25):22788-96). Furthermore, LRP-11 has been shown to have a potential role as a mediator of cellular drug uptake (Chung N S & Wasan K M 2004, Adv Drug Deliv Rev 56(9):1315-34) and has also been shown to be associated with Alzheimer's disease (Scherzer et al. 2004 Arch Neurol 61(8):1200-5), neuropeptide head activator signalling and function (Lintzel J et al. 2002 Biol Chem 383(11):1727-33) and atherosclerosis (Bujo H & Saito Y, 2000 J Atheroscler Thromb 7(1):21-5).

The MANSC domain has been noted in five classes of protein: i) proteins with unknown function containing a signal peptide and transmembrane region, ii) LRP-11 and similar proteins with a signal peptide, iii) uncharacterised proteins with signal peptide, low-density lipoprotein domain class A (LDLa) and transmembrane region, iv) HAI-1 proteins with signal peptide, two tandem repeats of Kunitz domains, LDLa and transmembrane region and v) other uncharacterised proteins with epidermal growth factor-like domain. It therefore appears that the MANSC domain is likely to be involved in development, regeneration of tissue following injury, Alzheimer's disease, atherosclerosis, cancer, tumour invasion and metastasis.

Increasing knowledge of proteins of the type described above is therefore of extreme importance in increasing the understanding of the underlying pathways that lead to the disease states and associated disease states mentioned above, and in developing more effective gene and/or drug therapies to treat these disorders.

### The Invention

The invention is based on the discovery of two novel Cys-rich, cell surface glycoproteins (INSP206 and INSP208) that can be functionally active as transmembrane, receptor proteins recognising one or more ligands by means of their extracellular regions on the cell surface. Alternatively, these latter regions may also have functionally active as soluble proteins that can bind these ligands in an extracellular space, such as in the circulation, in the peritoneum, or in the perivascular/peritubular space. Additionally, INSP208 possesses a MANSC domain.

It is predicted that the INSP206 polypeptide has three splice variants, referred to herein as “the INSP206SV1 full protein sequence”, “the INSP206SV2 full protein sequence” and “the INSP206SV3 full protein sequence”.

INSP206SV1 is identical to the predicted INSP206 sequence except that it contains an 18 bp pair deletion in the original prediction. The splice variant is predicted to be membrane anchored. The constituent amino acid sequences of this splice variant are recited in SEQ ID NO:26 (the “INSP206SV1 full protein sequence”), SEQ ID NO:32 (the “INSP206SV1 extracellular region protein sequence”) and SEQ ID NO:40 (the “INSP206SV mature extracellular protein sequence”).

The INSP206SV2 full protein sequence is identical to INSP206 except it contains a single nucleotide insertion (G) at the 3′ end of the cloned sequence, leading to a frame shift. The result of the frame shift is that the sequence of the last 4 amino acids encoded in the cloned fragment changes from SAVL to KCCV. The splice variant is predicted to be membrane anchored. The constituent amino acid sequence of this splice variant is recited in SEQ ID NO:28.

The INSP2060SV3 full protein sequence contains a 34 bp deletion in exon 1 leading to a prematurely truncated, naturally secreted form of the receptor containing 71 amino acids. The first 67 amino acids are identical to the INSP206 prediction. The constituent amino acid sequence of this splice variant is recited in SEQ ID NO:30.

It is also predicted that the INSP208 polypeptide has a splice variant, referred to herein as “the INSP208SV1 full protein sequence”. INSP208SV1 is identical to the predicted INSP208 sequence except for a 135 bp deletion (43aa) corresponding to exon 2. As this deletion removes 4 of the 8 predicted cysteines found in the extracellular domain, it is likely that the function of this variant will be disrupted. The constituent amino acid sequences of this splice variant are recited in SEQ ID NO:34, SEQ ID NO:36 and SEQ ID NO:38.

In one embodiment of the first aspect of the invention, there is provided a polypeptide which:
(i) comprises the amino acid sequence as recited in SEQ ID NO:2, SEQ ID NO:4, SEQ ID NO:6, SEQ ID NO:8, SEQ ID NO:10, SEQ ID NO:24, SEQ ID NO:26, SEQ ID NO:28, SEQ ID NO:30, SEQ ID NO:32 or SEQ ID NO:40;
(ii) comprises the amino acid sequence as recited in SEQ ID NO:12, SEQ ID NO:14, SEQ ID NO:16, SEQ ID NO:18, SEQ ID NO:20, SEQ ID NO:22, SEQ ID NO:34, SEQ ID NO:36 or SEQ ID NO:38;
(iii) is a fragment thereof which functions as a Cys-rich, cell surface glycoprotein, or has an antigenic determinant in common with the polypeptides of (i) or (ii); or
(iv) is a functional equivalent of (i), (ii) or (iii).

Preferably, the polypeptide according to this first aspect of the invention comprises or consists of the amino acid sequence as recited in SEQ ID NO:4, SEQ ID NO:14, SEQ ID NO:24, SEQ ID NO:32, SEQ ID NO:34 or SEQ ID NO:38.

According to a second embodiment of this first aspect of the invention, there is provided a polypeptide which consists of the amino acid sequence as recited in SEQ ID NO:2, SEQ ID NO:4, SEQ ID NO:6, SEQ ID NO:8, SEQ ID NO:10, SEQ ID NO:24, SEQ ID NO:26, SEQ ID NO:28, SEQ ID NO:30, SEQ ID NO:32 or SEQ ID NO:40.

According to a third embodiment of this first aspect of the invention, there is provided a polypeptide which consists of the amino acid sequence as recited in, SEQ ID NO:12, SEQ ID NO:14, SEQ ID NO:16, SEQ ID NO:18, SEQ ID NO:20, SEQ ID NO:22, SEQ ID NO:34, SEQ ID NO:36 or SEQ ID NO:38.

The polypeptide having the sequence recited in SEQ ID NO:2 is referred to hereafter as “INSP206 full protein sequence”. The polypeptide having the sequence recited in SEQ ID NO:4 is referred to hereafter as “INSP206 mature protein sequence”. The polypeptide having the sequence recited in SEQ ID NO:6 is referred to hereafter as the “INSP206 extracellular region protein sequence”. The polypeptide having the sequence recited in SEQ ID NO:8 is referred to hereafter as the “INSP206 mature extracellular region protein sequence”. The polypeptide having the sequence recited in SEQ ID NO:10 is referred to hereafter as the “INSP206 intracellular region protein sequence”. The polypeptide having the sequence recited in SEQ ID NO:24 is referred to hereafter as the “INSP206 cloned full protein sequence”. The polypeptide having the sequence recited in SEQ ID NO:26 is referred to hereafter as the “INSP206SV1 full protein sequence”. The polypeptide having the sequence recited in SEQ ID NO:28 is referred to hereafter as the “INSP206SV2 full protein sequence”. The polypeptide having the sequence recited in SEQ ID NO:30 is referred to hereafter as the “INSP206SV3 full length protein sequence”. The polypeptide having the sequence recited in SEQ ID NO:32 is referred to hereafter as the “INSP206SV1 extracellular region protein sequence”. The polypeptide having the sequence recited in SEQ ID NO:40 is referred to hereafter as the “INSP206SV1 mature extracellular region protein sequence”.

The polypeptide having the sequence recited in SEQ ID NO:12 is referred to hereafter as “INSP208 full protein sequence”. The polypeptide having the sequence recited in SEQ ID NO:14 is referred to hereafter as “INSP208 mature protein sequence”. The polypeptide having the sequence recited in SEQ ID NO:16 is referred to hereafter as “INSP208 extracellular region protein sequence”. The polypeptide having the sequence recited in SEQ ID NO:18 is referred to hereafter as “INSP208 mature extracellular region protein sequence”. The polypeptide having the sequence recited in SEQ ID NO:20 is referred to hereafter as “INSP208 MANSC domain protein sequence”. The polypeptide having the sequence recited in SEQ ID NO:22 is referred to hereafter as “INSP208 intracellular region protein sequence”. The polypeptide having the sequence recited in SEQ ID NO:34 is referred to hereafter as “INSP208SV1 full protein sequence”. The polypeptide having the sequence recited in SEQ ID NO:36 is referred to hereafter as “INSP208SV1 extracellular region protein sequence”. The polypeptide having the sequence recited in SEQ ID NO:38 is referred to hereafter as “INSP208SV1 mature extracellular region protein sequence” Although the Applicant does not wish to be bound by this theory, it is postulated that the first 20 amino acids of the INSP206 polypeptide form a signal peptide. The full length INSP206 polypeptide sequence without this postulated signal sequence is recited in SEQ ID NO: 4.

It is predicted herein that the INSP206 polypeptide contains a number of possible N-glycosylation sites. Accordingly, glycoproteins comprising or consisting of the INSP206 polypeptide, or fragments thereof, modified by N-glycosylation in either or both of the positions corresponding to Asn96, and Asn126 of SEQ ID NO:2 are included as aspects of the invention.

It is predicted herein that the INSP206 polypeptide contains a transmembrane domain between residues 178-200. Accordingly, extracellular variants of the INSP206 polypeptide are included as aspects of the invention. Preferred such variants include extracellular forms that comprise or consist of amino acid residues 1-177 or 21-177 of SEQ ID NO:2 or SEQ ID NO:24; amino acid residues 1-170 or 20-170 of SEQ ID NO:26; amino acid residues 1-176 or 19-176 of SEQ ID NO:28 in both their apo and N-glycosylated forms as herein described. The polypeptide having the sequence recited in SEQ ID NO:6 is referred to hereafter as “INSP206 extracellular region protein sequence”. The polypeptide having the sequence recited in SEQ ID NO:8 is referred to hereafter as “INSP206 mature extracellular region protein sequence” and excludes the amino acids that are predicted to form the INSP206 polypeptide signal peptide. The polypeptide having the sequence recited in SEQ ID NO:32 is referred to hereafter as the “INSP206SV1 extracellular region protein sequence”. The polypeptide having the sequence recited in SEQ ID NO:40 is referred to hereafter as the “INSP206SV1 mature extracellular region protein sequence”.

Polypeptide variants of this type are of particular utility in screening assays for ligands, such as secreted ligands that bind to the INSP206 polypeptides and to other proteins of this type. Such variants may also be used for quantification of such ligands, for example, in diagnosis of diseases in which these ligands play a role.

An intracellular variant of the INSP206 polypeptide is also included as an aspect of the invention. This variant is a polypeptide consisting of amino acid residues 201-239 inclusive of SEQ ID NO:2. The polypeptide having the sequence recited in SEQ ID NO:10 is referred to hereafter as “INSP206 intracellular region protein sequence”.

The term “INSP206 polypeptides” as used herein includes polypeptides comprising the INSP206 full protein sequence, the INSP206 mature protein sequence, the INSP206 extracellular region protein sequence, the INSP206 mature extracellular region protein sequence, the INSP206 intracellular region protein sequence, the INSP206 cloned full protein sequence, the INSP206SV1 full protein sequence, the INSP206SV2 full protein sequence, the INSP206SV3 full protein sequence, the INSP206SV1 extracellular region protein sequence and the INSP206SV1 mature extracellular region protein sequence.

Although the Applicant does not wish to be bound by this theory, it is postulated that the first 24 amino acids of the INSP208 polypeptide form a signal peptide. The full length INSP208 polypeptide sequence without this postulated signal sequence is recited in SEQ ID NO: 14.

It is predicted herein that the INSP208 polypeptide contains a number of possible N-glycosylation sites. Accordingly, glycoproteins comprising or consisting of the INSP208 polypeptide, or a fragment thereof, modified by N-glycosylation in 1, 2, 3, 4, 5, or all of the positions corresponding to Asn118, Asn187, Asn207, Asn229, Asn253 and Asn260 of SEQ ID NO:12 or positions Asn142, Asn162, Asn184, Asn208 and Asn215 of SEQ ID NO:34 are included as aspects of the invention.

It is predicted herein that the INSP208 polypeptide contains a transmembrane domain between residues 286-308. Accordingly, extracellular variants of the INSP208 polypeptide are included as aspects of the invention. Preferred such variants include extracellular forms that comprise or consist of amino acid residues 1-285 or 25-285 of SEQ ID NO:12 or amino acid residues 1-241 or 25-241 of SEQ ID NO:34, in both their apo and N-glycosylated forms as herein described. The polypeptide having the sequence recited in SEQ ID NO:16 is referred to hereafter as “INSP208 extracellular region protein sequence”. The polypeptide having the sequence recited in SEQ ID NO:18 is referred to hereafter as “INSP208 mature extracellular region protein sequence” and excludes the amino acids that are predicted to form the INSP208 polypeptide signal peptide. The polypeptide having the sequence recited in SEQ ID NO:36 is referred to hereafter as “INSP208SV1 extracellular region protein sequence” The polypeptide having the sequence recited in SEQ ID NO:38 is referred to hereafter as “INSP208SV1 mature extracellular region protein sequence”.

Polypeptide variants of this type are of particular utility in screening assays for ligands, such as secreted ligands that bind to the INSP208 polypeptides and to other proteins of this type. Such variants may also be used for quantification of such ligands, for example, in diagnosis of diseases in which these ligands play a role.

An intracellular variant of the INSP208 polypeptide is also included as an aspect of the invention. This variant is a polypeptide consisting of amino acid residues 309-340 inclusive of SEQ ID NO:12. The polypeptide having the sequence recited in SEQ ID NO:20 is referred to hereafter as “INSP206 intracellular region protein sequence”.

The term “INSP208 polypeptides” as used herein includes polypeptides comprising the INSP208 full protein sequence, the INSP208 mature protein sequence, the INSP208 extracellular region protein sequence, the INSP208 mature extracellular region protein sequence, the INSP208 MANSC domain protein sequence, the INSP208 intracellular region protein sequence, the INSP208 cloned full protein sequence, the INSP208SV1 full protein sequence, the INSP208SV1 extracellular region sequence and the INSP208SV1 mature extracellular region sequence.

Preferably, the term “Cys-rich, cell surface glycoprotein” may be a molecule matching the HMM build of the Pfam entry detected with an e-value lower than 0.1, 0.01, 0.001, 0.0001, 0.0002, 0.00001, 0.000001 or 0.0000001.

Preferably, a polypeptide according to the above embodiments of the first aspect of the invention functions as a secreted protein, particularly as a member of the Cys-rich, cell surface glycoprotein family. Knowledge of disease-specific glycoprotein structures and their functions may be used therapeutically, in immunotherapy, in blocking cell adhesion or interfering with other binding or biological processes (Brockhausen I et al., Acta Anat. 1998; 161(1-4):36-78; Bhatia P K and Mukhopadhyay A, Adv Biochem Eng Biotechnol. 1999; 64:155-201). Furthermore, the relationship between glycosylation/expression of glycoproteins and cancer progression/malignancy (Dennis J W et al., Biochim Biophys Acta. 1999 Dec. 6; 1473(1):21-34) or hereditary diseases (Dennis J W et al., Bioessays. 1999 May; 21(5):412-21) have been studied. In particular, diseased cells may have relative proportions of these structures that are often characteristically different from normal, and may be useful for the assessment of the stage of the disease and for diagnosis. Many cell surface glycoproteins are subjected to limited proteolysis by cellular proteases. This shedding mechanism allows secretion of extracellular domains that may affect the binding activities of other circulating proteins recognised by these extracellular regions. In fact, the shedding of extracellular domains, by altering cellular responses to exogenous stimuli, is involved in a number of pathophysiological processes, such as inflammation, cell degeneration and apoptosis, and oncogenesis, as intensively studied in cytokine/cytoldne receptors systems (Mullberg J et al., Eur Cytokine Netw. 2000 March; 11(1):27-38; Arribas J and Merlos-Suarez A Curr Top Dev Biol. 2003; 54:125-44; Dello Sbarba P and Rovida E; Biol Chem. 2002 January; 383(1):69-83).

Additionally, the INSP208 polypeptide, and fragments and functional equivalents thereof according to the above embodiments of the first aspect of the invention functions as a MANSC domain containing polypeptide. The MANSC domain is likely to be involved in development, regeneration of tissue following injury, Alzheimer's disease, atherosclerosis, cancer, tumour invasion and metastasis.

The polypeptides of the first aspect of the invention may further comprise a histidine tag. Preferably the histidine tag is found at the C-terminal of the polypeptide. Preferably the histidine tag comprises 1-10 histidine residues (e.g. 1, 2, 3, 4, 5, 6, 7, 8, 9 or 10 residues). More preferably the histidine tag comprises 6 histidine residues.

An “antigenic determinant” of the present invention may be a part of a polypeptide of the present invention, which binds to an antibody-combining site or to a T-cell receptor (TCR). Alternatively, an “antigenic determinant” may be a site on the surface of a polypeptide of the present invention to which a single antibody molecule binds. Generally an antigen has several or many different antigenic determinants and reacts with antibodies of many different specificities. Preferably, the antibody is immunospecific to a polypeptide of the invention. Preferably, the antibody is immunospecific to a polypeptide of the invention, which is not part of a fusion protein. Preferably, the antibody is immunospecific to INSP206, INSP208 or a fragment thereof. Antigenic determinants usually consist of chemically active surface groupings of molecules, such as amino acids or sugar side chains, and can have specific three dimensional structural characteristics, as well as specific charge characteristics. Preferably, the “antigenic determinant” refers to a particular chemical group on a polypeptide of the present invention that is antigenic, i.e. that elicit a specific immune response.

The following polypeptides, and their encoding nucleic acid sequences are specifically excluded from the scope of this invention: Q5V272 (herein referred to as SEQ ID NO:43), and ABN21508 (herein referred to as SEQ ID NO:44), ABG26371 (herein referred to as SEQ ID NO:45) and ACH72865 (herein referred to as SEQ ID NO:46). In a second aspect, the invention provides a purified nucleic acid molecule which encodes a polypeptide of the first aspect of the invention.

The term “purified nucleic acid molecule” preferably refers to a nucleic acid molecule of the invention that (1) has been separated from at least about 50 percent of proteins, lipids, carbohydrates, or other materials with which it is naturally found when total nucleic acid is isolated from the source cells, (2) is not linked to all or a portion of a polynucleotide to which the “purified nucleic acid molecule” is linked in nature, (3) is operably linked to a polynucleotide which it is not linked to in nature, or (4) does not occur in nature as part of a larger polynucleotide sequence. Preferably, the isolated nucleic acid molecule of the present invention is substantially free from any other contaminating nucleic acid molecule(s) or other contaminants that are found in its natural environment that would interfere with its use in polypeptide production or its therapeutic, diagnostic, prophylactic or research use. In a preferred embodiment, genomic DNA are specifically excluded from the scope of the invention. Preferably, genomic DNA larger than 10 kbp (kilo base pairs), 50 kbp, 100 kbp, 150 kbp, 200 kbp, 250 kbp or 300 kbp are specifically excluded from the scope of the invention. Preferably, the “purified nucleic acid molecule” consists of cDNA only.

Preferably, the purified nucleic acid molecule comprises the nucleic acid sequence as recited in SEQ ID NO:1 (encoding the INSP206 full protein sequence), SEQ ID NO:3 (encoding the INSP206 mature protein sequence), SEQ ID NO:5 (encoding the INSP206 extracellular region protein sequence), SEQ ID NO:7 (encoding the INSP206 mature extracellular region protein sequence), SEQ ID NO:9 (encoding the INSP206 intracellular region protein sequence), SEQ ID NO:11 (encoding the INSP208 full protein sequence), SEQ ID NO:13 (encoding the INSP208 mature protein sequence), SEQ ID NO:15 (encoding the INSP208 extracellular region protein sequence), SEQ ID NO:17 (encoding the INSP208 mature extracellular region protein sequence), SEQ ID NO:19 (encoding the INSP208 MANSC domain protein sequence), SEQ ID NO:21 (encoding the INSP208 intracellular region protein sequence), SEQ ID NO:23 (encoding the INSP206 cloned full protein sequence), SEQ ID NO:25 (encoding the INSP206SV1 full protein sequence), SEQ ID NO:27 (encoding the INSP206SV2 full protein sequence), SEQ ID NO: 29 (encoding the INSP206SV3 full protein sequence), SEQ ID NO:31 (encoding the INSP206SV1 extracellular region protein sequence), SEQ ID NO:33 (encoding the INSP208 cloned full protein sequence), SEQ ID NO:35 (encoding the INSP208SV1 full protein sequence), SEQ ID NO:37 (encoding the INSP208SV1 extracellular region protein sequence) or SEQ ID NO:39 (encoding the INSP206SV1 mature extracellular region protein sequence), or is a redundant equivalent or fragment of any one of these sequences.

The invention further provides that the purified nucleic acid molecule consists of the nucleic acid sequence as recited in SEQ ID NO:1 (encoding the INSP206 full protein sequence), SEQ ID NO:3 (encoding the INSP206 mature protein sequence), SEQ ID NO:5 (encoding the INSP206 extracellular region protein sequence), SEQ ID NO:7 (encoding the INSP206 mature extracellular region protein sequence), SEQ ID NO:9 (encoding the INSP206 intracellular region protein sequence), SEQ ID NO:11 (encoding the INSP208 full protein sequence), SEQ ID NO:13 (encoding the INSP208 mature protein sequence), SEQ ID NO:15 (encoding the INSP208 extracellular region protein sequence), SEQ ID NO:17 (encoding the INSP208 mature extracellular region protein sequence), SEQ ID NO:19 (encoding the INSP208 MANSC domain protein sequence) and SEQ ID NO:21 (encoding the INSP208 intracellular region protein sequence), SEQ ID NO:23 (encoding the INSP206 cloned full protein sequence), SEQ ID NO:25 (encoding the INSP206SV1 full protein sequence), SEQ ID NO:27 (encoding the INSP206SV2 full protein sequence), SEQ ID NO: 29 (encoding the INSP206SV3 full protein sequence), SEQ ID NO:31 (encoding the INSP206SV1 extracellular region protein sequence), SEQ ID NO:33 (encoding the INSP208 cloned full protein sequence), SEQ ID NO:35 (encoding the INSP208SV1 full protein sequence), SEQ ID NO:37 (encoding the INSP208SV1 extracellular region protein sequence) or SEQ ID NO:39 (encoding the INSP206SV1 mature extracellular region protein sequence), or is a redundant equivalent or fragment of any one of these sequences.

In a third aspect, the invention provides a purified nucleic acid molecule which hybridises under high stringency conditions with a nucleic acid molecule of the second aspect of the invention. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to members of the Cys-rich, cell surface glycoprotein family of the first aspect of the invention. Preferably, the ligand inhibits the function of a polypeptide of the first aspect of the invention which functions as a Cys-rich, cell surface glycoprotein. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

Such compounds may be identified using the assays and screening methods disclosed herein.

A compound of the seventh aspect of the invention may either increase (agonise) or decrease (antagonise) the level of expression of the gene or the activity of the polypeptide.

Importantly, the identification of the function of the INSP206 and INSP208 polypeptides allows for the design of screening methods capable of identifying compounds that are effective in the treatment and/or diagnosis of disease. Extracellular and intracellular forms of the INSP206 and INSP208 polypeptides are likely to be of particular utility in screening methods of this nature. Ligands and compounds according to the sixth and seventh aspects of the invention may be identified using such methods. These methods are included as aspects of the present invention.

Another aspect of this invention resides in the use of an INSP206 or INSP208 gene or polypeptide as a target for the screening of candidate drug modulators, particularly candidate drugs active against Cys-rich, cell surface glycoprotein related disorders.

A further aspect of this invention resides in methods of screening of compounds for therapy of Cys-rich, cell surface glycoprotein related disorders, comprising determining the ability of a compound to bind to an INSP206 or INSP208 gene or polypeptide, or a fragment thereof.

A further aspect of this invention resides in methods of screening of compounds for therapy of Cys-rich, cell surface glycoprotein related disorders, comprising testing for modulation of the activity of an INSP206 or INSP208 gene or polypeptide, or a fragment thereof.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis of diseases in which members of the Cys-rich, cell surface glycoprotein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy and allergic diseases, inflammatory bowel disease, arthritis, including osteoarthritis, inflammatory skin diseases, including psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions. Preferably, the diseases are those in which Cys-rich, cell surface glycoproteins are implicated. These molecules may also be used in the manufacture of a medicament for the treatment of such diseases. These molecules may also be used in contraception or for the treatment of reproductive disorders including infertility.

Preferably the diseases according to this aspect of the invention include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; inflammatory disorders, including allergy and allergic diseases, inflammatory bowel disease, arthritis, including osteoarthritis and inflammatory skin diseases, including psoriasis. More preferably the diseases include psoriasis and/or osteoarthritis

Although the Applicant does not wish to be bound by this theory, it is postulated that the polypeptides of the invention, preferably INSP206, have a role in allergic disease. The expression data disclosed herein support this theory by showing expression of INSP206 in leukocytic cell lines, notably granulocytes, eosinophils and basophils. Furthermore, the expression of INSP206 in two osteoarthritis samples and in inflammatory bowel disease samples indicates that the polypeptides of the invention, in particular INSP206 are also involved in these disease states.

Although the Applicant does not wish to be bound by this theory, it is postulated that the polypeptides of the invention, preferably INSP208, have a role in inflammatory skin diseases. The expression data disclosed herein support this theory by showing expression of INSP208 in psoriasis biopsy samples as well as in granulocytic cells. Furthermore, the expression data disclosed herein indicate that the polypeptides of the invention, preferably INSP208, may also attenuate and alleviate stress symptoms.

The moieties of the present invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may have particular utility in the therapy or diagnosis of disorders/diseases (the two terms are used interchangeably herein) of the disease states described above.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease.

Such a method will preferably be carried out in vitro. Similar methods may be used for monitoring the therapeutic treatment of disease in a patient, wherein altering the level of expression or activity of a polypeptide or nucleic acid molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridisation with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect, the invention provides for the use of a polypeptide of the first aspect of the invention as a Cys-rich, cell surface glycoprotein family. Suitable uses of the polypeptides of the invention as Cys-rich, cell surface glycoproteins include use as a regulator of cellular growth, metabolism or differentiation, use as part of a receptor/ligand pair and use as a diagnostic marker for a physiological or pathological condition.

In an eleventh aspect, the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease, including, but not limited to, cell proliferative disorders, including neoplasm, melanoma, lung, colorectal, breast, pancreas, head and neck and other solid tumours; myeloproliferative disorders such as leukemia, lymphoma, myelodysplastic syndromes and carcinoma, neoplasm, melanoma, lung, colorectal, breast, pancreas, head and neck and other solid tumours, blood disorders such as macroglobulinemia, autoimmune disease and inflammatory disorders, including allergy and allergic diseases, inflammatory bowel disease, arthritis, including osteoarthirtis, inflammatory skin diseases, including psoriasis, multiple sclerosis and respiratory tract inflammation, asthma, and organ transplant rejection, B-cell disorders, cardiovascular disorders, neurological disorders, developmental disorders, fertility disorders, metabolic disorders, AIDS, renal disease, infections and other pathological conditions.

Preferably the medicament according to this aspect of the invention is used to treat; cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; inflammatory disorders, including allergy and allergic diseases, inflammatory bowel disease, arthritis, including osteoarthritis and inflammatory skin diseases, including psoriasis. More preferably the medicament is for the treatment of psoriasis and/or osteoarthritis

In a thirteenth aspect, the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient, the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist. Examples of such antagonists include antisense nucleic acid molecules, ribozymes and ligands, such as antibodies.

The INSP206 or INSP208 polypeptides are Cys-rich, cell surface glycoproteins and thus have roles in many disease states. Antagonists of the INSP206 or INSP208 polypeptides are of particular interest as they provide a way of modulating these disease states.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

As used herein, “functional equivalent” refers to a protein or nucleic acid molecule that possesses functional or structural characteristics that are substantially similar to a polypeptide or nucleic acid molecule of the present invention. A functional equivalent of a protein may contain modifications depending on the necessity of such modifications for the performance of a specific function. The term “functional equivalent” is intended to include the fragments, mutants, hybrids, variants, analogs, or chemical derivatives of a molecule.

Preferably, the “functional equivalent” may be a protein or nucleic acid molecule that exhibits any one or more of the functional activities of the polypeptides of the present invention.

Preferably, the “functional equivalent” may be a protein or nucleic acid molecule that displays substantially similar activity compared with INSP206, INSP208 or fragments thereof in a suitable assay for the measurement of biological activity or function. Preferably, the “functional equivalent” may be a protein or nucleic acid molecule that displays identical or higher activity compared with INSP206, INSP208 or fragments thereof in a suitable assay for the measurement of biological activity or function. Preferably, the “functional equivalent” may be a protein or nucleic acid molecule that displays 50%, 60%, 70%, 80%, 90%, 95%, 98%, 99%, 100% or more activity compared with INSP206, INSP208 or fragments thereof in a suitable assay for the measurement of biological activity or function.

Preferably, the “functional equivalent” may be a protein or polypeptide capable of exhibiting a substantially similar in vivo or in vitro activity as the polypeptides of the invention. Preferably, the “functional equivalent” may be a protein or polypeptide capable of interacting with other cellular or extracellular molecules in a manner substantially similar to the way in which the corresponding portion of the polypeptides of the invention would. For example, a “functional equivalent” would be able, in an immunoassay, to diminish the binding of an antibody to the corresponding peptide (i.e., the peptide the amino acid sequence of which was modified to achieve the “functional equivalent”) of the polypeptide of the invention, or to the polypeptide of the invention itself, where the antibody was raised against the corresponding peptide of the polypeptide of the invention. An equimolar concentration of the functional equivalent will diminish the aforesaid binding of the corresponding peptide by at least about 5%, preferably between about 5% and 10%, more preferably between about 10% and 25%, even more preferably between about 25% and 50%, and most preferably between about 40% and 50%.

For example, functional equivalents can be fully functional or can lack function in one or more activities. Thus, in the present invention, variations can affect the function, for example, of the activities of the polypeptide that reflect its identity as a Cys-rich, cell surface glycoprotein.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984); Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

As used herein, the term “polypeptide” includes any peptide or protein comprising two or more amino acids joined to each other by peptide bonds or modified peptide bonds, i.e. peptide isosteres. This term refers both to short chains (peptides and oligopeptides) and to longer chains (proteins).

The polypeptide of the present invention may be in the form of a mature protein or may be a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

The polypeptide of the first aspect of the invention may form part of a fusion protein. For example, it is often advantageous to include one or more additional amino acid sequences which may contain secretory or leader sequences, pro-sequences, sequences which aid in purification, or sequences that confer higher protein stability, for example during recombinant production. Alternatively or additionally, the mature polypeptide may be fused with another compound, such as a compound to increase the half-life of the polypeptide (for example, polyethylene glycol).

In a further preferred embodiment, a polypeptide of the invention, that may comprise a sequence having at least 85% of homology with INSP206 or INSP208, is a fusion protein.

These fusion proteins can be obtained by cloning a polynucleotide encoding a polypeptide comprising a sequence having at least 85% of homology with INSP206 or INSP208 in frame to the coding sequences for a heterologous protein sequence.

The term “heterologous”, when used herein, is intended to designate any polypeptide other than a human INSP206 or INSP208 polypeptide. Examples of heterologous sequences, that can be comprised in the fusion proteins either at the N- or C-terminus, include: extracellular domains of membrane-bound protein, immunoglobulin constant regions (Fc regions), multimerization domains, domains of extracellular proteins, signal sequences, export sequences, and sequences allowing purification by affinity chromatography.

Many of these heterologous sequences are commercially available in expression plasmids since these sequences are commonly included in fusion proteins in order to provide additional properties without significantly impairing the specific biological activity of the protein fused to them (Terpe K, 2003, Appl Microbiol Biotechnol, 60:523-33). Examples of such additional properties are a longer lasting half-life in body fluids, the extracellular localization, or an easier purification procedure as allowed by the a stretch of Histidines forming the so-called “histidine tag” (Gentz et al. 1989, Proc Natl Acad Sci USA, 86:821-4) or by the “HA” tag, an epitope derived from the influenza hemagglutinin protein (Wilson et al. 1994, Cell, 37:767-78). If needed, the heterologous sequence can be eliminated by a proteolytic cleavage, for example by inserting a proteolytic cleavage site between the protein and the heterologous sequence, and exposing the purified fusion protein to the appropriate protease. These features are of particular importance for the fusion proteins since they facilitate their production and use in the preparation of pharmaceutical compositions. For example, the INSP206 or INSP208 polypeptide may be purified by means of a hexa-histidine peptide fused at the C-terminus of INSP206 or INSP208. When the fusion protein comprises an immunoglobulin region, the fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met (SEQ ID NO:78) introduced between the sequence of the substances of the invention and the immunoglobulin sequence. The resulting fusion protein has improved properties, such as an extended residence time in body fluids (i.e. an increased half-life), increased specific activity, increased expression level, or the purification of the fusion protein is facilitated.

In a preferred embodiment, the protein is fused to the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG2 or IgG4, or other Ig classes, like IgM or IgA, for example. Fusion proteins may be monomeric or multimeric, hetero- or homomultimeric.

In a further preferred embodiment, the functional derivative comprises at least one moiety attached to one or more functional groups, which occur as one or more side chains on the amino acid residues. Preferably, the moiety is a polyethylene (PEG) moiety. PEGylation may be carried out by known methods, such as the ones described in WO99/55377, for example.

Polypeptides may contain amino acids other than the 20 gene-encoded amino acids, modified either by natural processes, such as by post-translational processing or by chemical modification techniques which are well known in the art. Among the known modifications which may commonly be present in polypeptides of the present invention are glycosylation, lipid attachment, sulphation, gamma-carboxylation, for instance of glutamic acid residues, hydroxylation and ADP-ribosylation. Other potential modifications include acetylation, acylation, amidation, covalent attachment of flavin, covalent attachment of a haeme moiety, covalent attachment of a nucleotide or nucleotide derivative, covalent attachment of a lipid derivative, covalent attachment of phosphatidylinositol, cross-linking, cyclization, disulphide bond formation, demethylation, formation of covalent cross-links, formation of cysteine, formation of pyroglutamate, formylation, GPI anchor formation, iodination, methylation, myristoylation, oxidation, proteolytic processing, phosphorylation, prenylation, racemization, selenoylation, transfer-RNA mediated addition of amino acids to proteins such as arginylation, and ubiquitination.

Modifications can occur anywhere in a polypeptide, including the peptide backbone, the amino acid side-chains and the amino or carboxyl termini. In fact, blockage of the amino or carboxyl terminus in a polypeptide, or both, by a covalent modification is common in naturally-occurring and synthetic polypeptides and such modifications may be present in polypeptides of the present invention.

The modifications that occur in a polypeptide often will be a function of how the polypeptide is made. For polypeptides that are made recombinantly, the nature and extent of the modifications in large part will be determined by the post-translational modification capacity of the particular host cell and the modification signals that are present in the amino acid sequence of the polypeptide in question. For instance, glycosylation patterns vary between different types of host cell.

The polypeptides of the present invention can be prepared in any suitable manner. Such polypeptides include isolated naturally-occurring polypeptides (for example purified from cell culture), recombinantly-produced polypeptides (including fusion proteins), synthetically-produced polypeptides or polypeptides that are produced by a combination of these methods.

The functionally-equivalent polypeptides of the first aspect of the invention may be polypeptides that are homologous to the INSP206 or INSP208 polypeptides. Two polypeptides are said to be “homologous”, as the term is used herein, if the sequence of one of the polypeptides has a high enough degree of identity or similarity to the sequence of the other polypeptide. “Identity” indicates that at any particular position in the aligned sequences, the amino acid residue is identical between the sequences. “Similarity” indicates that, at any particular position in the aligned sequences, the amino acid residue is of a similar type between the sequences. Degrees of identity and similarity can be readily calculated (Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

Homologous polypeptides therefore include natural biological variants (for example, allelic variants or geographical variations within the species from which the polypeptides are derived) and mutants (such as mutants containing amino acid substitutions, insertions or deletions) of the INSP206 and INSP208 polypeptides. Such mutants may include polypeptides in which one or more of the amino acid residues are substituted with a conserved or non-conserved amino acid residue preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

In this case, preferred homologues of the INSP206 polypeptide are defined as those polypeptides which have an E-value of 10⁻² or less, preferably 10⁻⁵ or less, more preferably 10⁻¹⁰ or less, even more preferably 10⁻⁵⁰ or less, when the sequence profile given in Table 1 is input as a query sequence into BLAST version 2.1.3, using the default parameters specified by the NCBI (the National Center for Biotechnology Information; http://www.ncbi.nlm.nih.gov/) [Blosum 62 matrix; gap open penalty=11 and gap extension penalty=1].

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physico-chemical studies on the sequence and/or the structure of proteins (Rogov S I and Nekrasov A N, 2001). Protein design experiments have shown that the use of specific subsets of amino acids can produce foldable and active proteins, helping in the classification of amino acid “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table 3.

Specific, non-conservative mutations can be also introduced in the polypeptides of the invention with different purposes. Mutations reducing the affinity of the Cys-rich, cell surface glycoprotein may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Preferred alternative, synonymous groups for amino acids derivatives included in peptide mimetics are those defined in Table 4. A non-exhaustive list of amino acid derivatives also include aminoisobutyric acid (Aib), hydroxyproline (Hyp), 1,2,3,4-tetrahydro-isoquinoline-3-COOH, indoline-2-carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem A G, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design”, edited by Veerapandian P, Marcel Dekker Inc., pg. 557-663, 1997).

TABLE 1

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | G | −1 | −3 | −1 | −1 | −4 | −2 | −2 | 6 | −3 | −5 | −5 | −2 | −3 | −4 | 2 | −1 | −2 | −3 | −4 | −3 |
| 2 | C | 0 | −4 | −4 | −4 | 10 | −4 | −5 | −4 | −4 | −1 | −1 | −4 | −1 | −3 | −4 | −1 | −1 | −3 | −3 | −1 |
| 3 | L | −1 | −3 | −4 | −4 | −1 | −3 | −3 | −5 | −4 | 2 | 4 | −3 | 0 | −1 | −3 | −3 | −1 | −3 | −1 | 2 |
| 4 | E | −2 | 0 | −1 | 0 | −3 | 4 | 3 | −3 | 3 | 0 | −2 | 1 | −1 | −3 | −2 | −1 | −2 | −3 | −1 | −1 |
| 5 | C | 0 | −4 | −4 | −4 | 10 | −4 | −5 | −4 | −4 | −1 | −1 | −4 | −1 | −3 | −4 | −1 | −1 | −3 | −3 | −1 |
| 6 | D | −3 | −2 | 0 | 7 | −4 | 0 | 1 | −2 | 3 | −4 | −5 | −1 | −4 | −3 | −2 | −1 | −2 | −4 | −2 | −4 |
| 7 | P | −1 | −1 | −1 | −1 | −3 | 3 | 0 | −2 | −2 | −4 | −3 | −1 | −2 | −4 | 6 | 0 | −1 | −4 | −3 | −3 |
| 8 | K | −1 | 0 | 3 | −1 | −3 | −1 | −1 | −2 | −1 | −2 | 0 | 2 | −1 | 2 | −2 | 2 | −1 | −3 | −1 | −2 |
| 9 | F | −1 | −4 | −4 | −4 | −2 | −3 | −3 | −4 | −2 | 1 | 0 | −3 | 0 | 5 | −3 | −3 | −1 | −1 | 0 | 4 |
| 10 | I | 0 | −2 | −2 | −3 | −1 | −2 | −2 | −3 | −3 | 1 | 2 | −2 | 0 | −1 | −2 | 0 | 3 | −3 | −2 | 1 |
| 11 | E | −2 | −1 | −1 | 2 | −3 | 0 | 4 | −3 | −1 | −1 | 1 | 1 | −1 | −2 | −2 | −1 | −1 | −3 | −2 | −1 |
| 12 | D | 3 | 0 | −1 | 3 | −2 | 0 | 1 | −1 | −2 | −3 | −3 | 2 | −2 | −3 | −1 | 0 | −1 | −4 | −3 | −2 |
| 13 | V | −1 | −3 | −4 | −4 | −2 | −3 | −3 | −4 | −3 | 1 | 3 | −3 | 0 | 3 | −4 | −3 | −1 | −2 | 0 | 2 |
| 14 | G | −1 | 3 | 0 | −1 | −3 | 0 | 0 | 3 | −1 | −4 | −3 | 3 | −2 | −3 | −2 | 2 | −1 | −3 | −3 | −3 |
| 15 | S | −1 | −1 | 0 | −1 | −2 | 1 | −1 | −2 | 3 | −2 | −2 | −1 | −1 | 3 | −2 | 2 | 3 | −2 | 0 | −2 |
| 16 | L | −2 | −3 | −3 | −4 | −2 | −2 | −3 | −4 | −1 | 0 | 4 | −3 | 0 | 3 | −4 | −3 | −2 | −1 | 4 | 0 |
| 17 | L | −1 | 4 | −1 | −1 | −3 | 0 | 3 | −3 | −1 | −1 | 1 | 0 | −1 | −3 | −2 | −1 | −1 | −3 | −2 | −2 |
| 18 | G | −1 | −1 | 2 | 1 | −3 | −1 | 0 | 3 | 5 | −4 | −4 | 1 | −3 | −3 | −2 | 1 | −1 | −3 | −1 | −3 |
| 19 | N | 2 | −1 | 3 | 2 | −2 | −1 | 0 | −1 | 5 | −3 | −3 | −1 | −2 | −3 | −2 | 0 | 1 | −4 | −1 | −2 |
| 20 | L | −1 | −3 | −3 | −4 | −2 | −2 | −3 | −4 | −1 | 2 | 3 | −3 | 0 | 0 | −3 | −3 | −1 | −1 | 4 | 2 |
| 21 | I | −1 | −2 | 3 | −2 | −2 | −2 | −2 | −3 | −2 | 3 | 2 | −2 | 0 | −1 | −3 | −2 | −1 | −4 | −2 | 3 |
| 22 | P | −2 | −2 | 1 | −2 | −3 | −2 | −2 | −2 | −2 | −2 | 0 | −2 | −1 | 1 | 6 | −1 | 1 | −3 | −2 | −2 |
| 23 | S | −1 | 0 | 0 | 1 | −3 | 0 | 0 | 1 | −2 | −2 | 0 | 3 | −1 | −3 | −2 | 2 | −1 | −3 | −3 | −2 |
| 24 | E | −1 | 3 | 0 | 0 | −3 | 0 | 2 | −2 | 6 | −3 | −3 | 0 | −2 | −3 | −2 | 2 | −1 | −3 | −1 | −3 |
| 25 | V | −2 | −3 | −4 | −4 | −2 | −3 | −3 | −4 | −3 | 0 | 2 | −3 | 0 | 4 | −4 | −3 | −2 | 7 | 0 | 2 |
| 26 | P | −2 | −2 | −1 | 1 | −3 | 1 | 0 | −2 | 3 | −4 | −3 | −1 | −2 | −2 | 5 | −2 | −2 | 7 | −1 | −3 |
| 27 | G | 0 | −3 | −1 | 3 | −2 | −1 | 0 | 2 | −2 | 0 | −1 | −2 | 0 | −2 | −2 | −1 | −1 | −3 | −2 | 3 |
| 28 | R | 0 | 2 | −1 | −3 | −2 | −1 | −2 | 2 | 3 | −1 | 1 | −1 | −1 | −2 | −3 | −1 | −2 | −3 | −1 | −2 |
| 29 | T | −2 | −1 | 2 | 4 | −4 | 0 | 3 | −2 | −1 | −3 | −3 | 1 | −3 | −3 | −2 | 0 | 1 | −4 | −3 | −3 |
| 30 | Q | −1 | −1 | −1 | −2 | −3 | 4 | 0 | 0 | 4 | 3 | −1 | −1 | 0 | −2 | −2 | −1 | −2 | −3 | −1 | 0 |
| 31 | L | −2 | −2 | −2 | −3 | −2 | −2 | −2 | −3 | 5 | 0 | 3 | −2 | 0 | −1 | 3 | −2 | −2 | −3 | −1 | −1 |
| 32 | L | −1 | 0 | −2 | −2 | −2 | 3 | 0 | −3 | −2 | 0 | 1 | 3 | 0 | −2 | −2 | −1 | −1 | −3 | −2 | 2 |
| 33 | E | 3 | −1 | 2 | 1 | −2 | 0 | 1 | −1 | −1 | −2 | −2 | −1 | −2 | −3 | −2 | 2 | 0 | −4 | −3 | −2 |
| 34 | R | −1 | 4 | −1 | −3 | −3 | 0 | −1 | 2 | −1 | −1 | −1 | 0 | 3 | −3 | −3 | −1 | −2 | −3 | −2 | 0 |
| 35 | Q | 3 | −1 | −2 | −2 | −1 | 3 | −1 | −2 | −2 | −1 | 0 | −1 | 6 | −2 | −2 | 0 | −1 | −2 | −2 | 0 |
| 36 | I | −1 | 1 | −1 | −1 | −3 | 1 | 1 | 1 | 3 | 1 | 1 | −1 | 0 | −2 | −3 | −1 | −2 | −3 | −1 | −1 |
| 37 | K | 2 | 2 | −2 | −3 | −2 | −1 | −1 | −2 | −2 | −1 | 1 | 2 | 3 | −2 | −2 | −1 | −1 | −3 | −2 | −1 |
| 38 | E | −1 | −2 | −2 | −1 | −2 | −1 | 2 | −3 | −2 | 0 | 0 | −1 | 0 | −2 | −2 | 0 | 1 | −3 | −2 | 3 |
| 39 | M | −2 | −2 | 2 | 3 | −3 | −1 | 1 | −3 | −2 | 1 | 2 | −1 | 2 | −2 | −2 | −1 | −1 | −3 | −2 | 0 |
| 40 | I | −2 | −2 | 2 | −2 | −2 | −2 | −2 | −3 | 3 | 1 | 0 | −2 | 3 | −1 | −3 | −1 | 1 | 7 | 0 | 0 |
| 41 | H | 1 | −1 | 0 | −1 | −2 | −1 | 1 | 1 | 5 | 0 | −2 | −1 | −2 | −2 | −2 | 2 | −1 | −3 | −1 | −1 |
| 42 | L | −1 | −2 | −2 | 1 | −2 | 1 | −1 | −3 | −3 | 2 | 1 | −2 | 4 | −1 | −2 | −2 | −1 | −3 | −2 | 3 |
| 43 | S | −1 | −1 | 2 | 2 | −3 | 0 | 3 | −1 | −1 | −3 | −3 | 1 | −2 | −3 | −2 | 3 | 0 | −4 | −3 | −3 |
| 44 | F | −1 | −2 | −1 | −1 | −2 | −1 | 1 | −3 | −2 | 0 | −1 | −2 | −1 | 5 | −3 | 0 | 2 | −1 | 0 | 0 |
| 45 | K | −1 | 3 | −2 | −3 | −2 | 0 | −1 | −3 | −2 | 0 | 1 | 3 | 4 | −2 | −2 | −1 | −1 | −3 | −2 | 1 |
| 46 | V | −1 | −1 | 0 | 3 | −2 | −1 | 0 | −2 | −2 | 0 | −2 | 2 | −1 | −3 | −2 | 1 | 1 | −4 | −2 | 2 |
| 47 | S | −1 | −2 | −1 | −1 | −2 | 0 | 2 | −2 | 0 | −1 | 0 | −1 | −1 | 0 | −2 | 3 | −1 | −1 | 4 | −2 |
| 48 | H | 3 | −1 | 2 | −2 | −2 | −1 | −1 | −2 | 5 | −1 | 1 | −2 | −1 | −2 | −2 | 0 | −1 | −3 | −1 | −1 |
| 49 | S | −1 | 0 | 0 | 0 | −3 | 0 | 3 | −3 | 5 | −2 | 0 | 1 | 2 | −2 | −2 | 0 | −1 | −3 | −1 | −2 |
| 50 | D | −2 | −2 | 4 | 5 | −3 | −1 | 0 | −2 | −1 | −1 | 1 | −1 | −1 | −3 | −2 | 0 | −1 | −4 | −3 | −2 |
| 51 | K | 3 | −1 | −1 | −2 | −1 | −1 | −1 | −1 | −2 | −1 | −1 | 2 | −1 | −3 | −2 | 1 | 0 | −4 | −2 | 1 |
| 52 | R | −2 | 0 | −3 | −3 | −2 | −2 | −3 | −4 | −1 | 2 | 0 | −2 | 2 | 4 | −4 | −2 | −2 | 0 | 5 | 0 |
| 53 | L | −1 | −3 | −3 | −3 | −2 | −2 | −3 | −4 | −3 | 0 | 3 | −2 | 3 | −1 | 3 | −2 | −1 | −3 | −2 | 2 |
| 54 | R | 2 | 2 | −1 | −2 | −3 | −1 | −2 | 5 | −2 | −3 | −3 | −1 | −2 | −3 | −2 | 0 | −2 | −3 | −3 | −3 |
| 55 | V | 0 | 0 | −2 | −2 | −2 | 0 | 1 | −3 | −2 | 0 | 0 | 4 | 0 | −2 | −2 | −1 | −1 | −3 | −2 | 2 |
| 56 | L | −1 | −3 | −4 | −4 | −1 | −3 | −3 | −4 | −4 | 3 | 2 | −3 | 0 | −1 | −3 | −3 | −1 | −3 | −1 | 4 |
| 57 | A | 2 | −2 | −1 | 3 | −2 | −1 | 1 | −2 | −2 | −1 | −2 | −1 | −2 | −3 | −2 | 0 | 2 | −4 | −3 | 0 |
| 58 | V | −1 | 2 | −1 | −1 | −3 | 0 | 3 | −3 | −2 | 0 | −1 | 0 | −1 | −3 | −2 | −1 | 1 | −3 | −2 | 3 |
| 59 | Q | 0 | 1 | 4 | 2 | −3 | 2 | 1 | −1 | −1 | −3 | −3 | 1 | −2 | −3 | −2 | 0 | −1 | −4 | −3 | −3 |
| 60 | Q | −1 | 0 | 2 | −1 | −3 | 4 | 0 | −2 | −1 | −3 | −2 | 3 | −1 | −3 | −2 | 0 | 3 | −3 | −2 | −2 |
| 61 | V | −1 | −3 | −4 | −4 | −1 | −3 | −3 | −4 | −4 | 1 | 4 | −3 | 1 | −1 | −3 | −3 | −1 | −3 | −1 | 2 |
| 62 | V | −2 | −1 | −1 | 3 | −3 | 2 | 3 | −3 | −1 | 1 | −2 | −1 | −1 | −1 | −2 | −1 | −1 | −2 | 2 | 0 |
| 63 | K | −1 | 0 | −1 | −1 | −3 | 4 | 0 | −3 | −1 | −2 | 0 | 5 | −1 | −3 | −2 | −1 | −1 | −3 | −2 | −2 |
| 64 | L | 1 | −3 | −3 | −3 | −2 | −2 | −3 | 1 | −3 | 1 | 2 | −2 | 0 | −2 | −3 | −2 | −1 | −3 | −2 | 4 |
| 65 | R | 3 | 3 | −1 | −2 | −1 | −1 | −1 | −1 | −2 | −2 | −2 | 0 | −1 | −3 | −2 | 1 | 1 | −3 | −3 | −1 |
| 66 | T | −1 | −2 | −1 | −1 | −2 | 0 | 2 | −2 | −2 | −3 | −2 | −1 | −2 | −2 | −2 | 1 | 3 | 8 | −1 | −2 |
| 67 | W | 2 | −2 | −1 | −2 | −2 | −1 | −2 | −1 | −2 | −2 | −2 | −2 | −1 | 2 | −2 | 3 | 0 | 8 | 0 | −2 |
| 68 | L | −1 | −3 | −4 | −4 | −2 | −3 | −3 | −4 | −3 | 1 | 3 | −3 | 0 | 4 | −3 | −3 | −1 | −2 | 0 | 3 |
| 69 | K | −2 | 0 | −2 | −3 | −3 | 0 | −1 | −3 | −1 | −1 | 1 | 4 | 0 | 0 | −2 | −1 | −2 | −1 | 4 | −1 |
| 70 | N | −2 | 0 | 5 | 2 | −4 | 3 | 0 | −1 | 0 | −4 | −3 | 3 | −2 | −4 | −2 | 0 | −1 | −4 | −2 | −3 |
| 71 | E | −2 | 1 | 0 | 3 | −4 | 3 | 3 | −2 | −1 | −3 | −2 | 0 | 2 | −3 | −2 | −1 | −1 | −3 | −2 | −2 |
| 72 | F | 0 | −2 | −3 | −3 | −2 | −2 | −3 | −3 | −3 | 0 | 2 | −2 | 4 | 3 | −3 | −1 | 1 | −1 | 0 | 0 |
| 73 | Y | −2 | 0 | −1 | 3 | −3 | 3 | 0 | −2 | 0 | −3 | −3 | 3 | −2 | −1 | −2 | −1 | −2 | −1 | 4 | −2 |
| 74 | K | −2 | 4 | 0 | −2 | −3 | 3 | 0 | −3 | 4 | −3 | −3 | 2 | −1 | −2 | −2 | −1 | −2 | −2 | 2 | −3 |
| 75 | L | −1 | −3 | −4 | −4 | −1 | −3 | −4 | −5 | −4 | 4 | 4 | −3 | 0 | 0 | −4 | −3 | −1 | −3 | −1 | 1 |
| 76 | G | −1 | −1 | −1 | −2 | −2 | −1 | −2 | 2 | −1 | −2 | −1 | 1 | 2 | 0 | −2 | −1 | 3 | −1 | 4 | −1 |
| 77 | N | 0 | −1 | 3 | 3 | −3 | 3 | 0 | 1 | −1 | −3 | −3 | −1 | −2 | −3 | −2 | 0 | −1 | −4 | −3 | −3 |
| 78 | E | −1 | −1 | 4 | 0 | −3 | 0 | 2 | 3 | −1 | −4 | −4 | 0 | −2 | −3 | −2 | 2 | −1 | −4 | −3 | −3 |
| 79 | T | −1 | −1 | 0 | 3 | −2 | 0 | 0 | −2 | −2 | −3 | −3 | 2 | −2 | −3 | −1 | 2 | 3 | −4 | −3 | −2 |
| 80 | W | −2 | −2 | −3 | −4 | −2 | −1 | −2 | −3 | −3 | 0 | 2 | 1 | 4 | −1 | −3 | −2 | −2 | 8 | 0 | 1 |
| 81 | K | −2 | 0 | −1 | −2 | −3 | 0 | 0 | −3 | −1 | −3 | −2 | 5 | −1 | −1 | −2 | −1 | 1 | −1 | 4 | −2 |
| 82 | G | −1 | −3 | −1 | 3 | −3 | −2 | −1 | 5 | −2 | −3 | −3 | −2 | −3 | 3 | −3 | −1 | −2 | −2 | −1 | −3 |
| 83 | V | −1 | −2 | −2 | 2 | −3 | 0 | 3 | −3 | −2 | 0 | −1 | −1 | −1 | −3 | 3 | −1 | −1 | −4 | −2 | −3 |
| 84 | F | −1 | −3 | −2 | −3 | −3 | −2 | −3 | 2 | −3 | −1 | 1 | −2 | 0 | 3 | 3 | −2 | −2 | −2 | 0 | −1 |
| 85 | I | −2 | −3 | −3 | −4 | −2 | −3 | −3 | −4 | −1 | 3 | 2 | −3 | 0 | 4 | −4 | −3 | −2 | 0 | 4 | 0 |
| 86 | Y | −2 | −3 | −3 | −4 | −2 | −3 | −3 | −4 | −2 | 3 | 2 | −3 | 0 | 4 | −4 | −3 | −2 | −1 | 3 | 2 |
| 87 | Q | −1 | 0 | −1 | 0 | −4 | 3 | 3 | −3 | −1 | −4 | −3 | 3 | −2 | −4 | 3 | −1 | −1 | −4 | −2 | −3 |
| 88 | G | −1 | −1 | 5 | 0 | −3 | −1 | −1 | 4 | 0 | −4 | −4 | −1 | −3 | −3 | −2 | 0 | −1 | −3 | −3 | −3 |
| 89 | K | −2 | 0 | 2 | 0 | −4 | 0 | 6 | −2 | 0 | −4 | −3 | 2 | −2 | −4 | −2 | 0 | −1 | −4 | −3 | −3 |
| 90 | L | −1 | −3 | −4 | −5 | −1 | −3 | −4 | −5 | −4 | 1 | 5 | −3 | 1 | 0 | −4 | −3 | −1 | −3 | −1 | 0 |
| 91 | L | −1 | 2 | −3 | −4 | −2 | −2 | −2 | −4 | −2 | 0 | 3 | −1 | 0 | 2 | −3 | −2 | −1 | −2 | −1 | 2 |
| 92 | D | 0 | −2 | 1 | 1 | −2 | −1 | 1 | −2 | −1 | −3 | −3 | −1 | −2 | −2 | −2 | 1 | 1 | 8 | −1 | −2 |
| 93 | V | −1 | −3 | −3 | −4 | −2 | −2 | −3 | −4 | −3 | 4 | 2 | −3 | 5 | 2 | −3 | −2 | −1 | −2 | −1 | 2 |
| 94 | C | −2 | 4 | −2 | −3 | 4 | −1 | −2 | −3 | −2 | 0 | 2 | 0 | 0 | 3 | −3 | −2 | −2 | −2 | 0 | −1 |
| 95 | Q | 0 | 4 | 0 | −1 | −3 | 3 | 1 | −2 | 4 | −3 | −3 | 0 | −1 | −3 | −2 | −1 | −1 | −3 | −1 | −3 |
| 96 | N | −2 | −1 | 4 | 0 | −3 | 0 | 2 | −2 | 6 | −2 | 0 | −1 | −2 | −2 | −2 | 0 | −1 | −3 | −1 | −2 |
| 97 | L | 0 | −1 | −2 | −2 | −2 | 5 | 0 | −3 | −2 | 0 | 1 | 0 | 0 | −2 | −2 | −1 | −1 | −3 | −2 | 1 |
| 98 | E | −1 | −1 | −2 | −1 | −3 | 0 | 2 | −3 | −2 | 3 | 0 | 3 | 0 | −2 | −2 | −1 | −1 | −4 | −2 | 2 |
| 99 | S | −1 | 2 | 0 | 2 | −3 | 0 | 2 | −2 | 3 | −3 | −3 | 4 | −2 | −3 | −2 | 1 | −1 | −4 | −2 | −3 |
| 100 | K | −1 | 0 | −1 | −1 | −3 | 0 | 2 | −3 | −2 | 1 | 1 | 2 | 0 | −2 | −2 | −1 | 1 | −3 | −2 | 0 |
| 101 | L | −2 | −3 | −4 | −4 | −2 | −3 | −4 | −4 | −3 | 3 | 3 | −3 | 0 | 5 | −4 | −3 | −2 | −1 | 0 | 0 |
| 102 | K | 0 | 0 | 2 | −1 | −3 | 3 | 0 | −2 | −1 | −3 | −2 | 4 | −1 | −3 | −2 | 0 | 1 | −3 | −2 | −2 |
| 103 | E | −1 | 0 | 4 | 0 | −3 | 2 | 1 | 1 | 0 | −3 | −3 | 1 | −2 | −3 | −2 | 0 | 1 | −3 | −3 | −3 |
| 104 | L | 3 | −2 | −3 | −3 | −1 | −2 | −2 | −2 | −2 | 1 | 2 | −2 | 0 | 0 | −3 | −1 | −1 | −2 | 3 | 1 |
| 105 | L | 0 | −3 | −3 | −4 | −1 | −3 | −3 | −4 | −3 | 4 | 3 | −3 | 0 | −1 | −3 | −2 | −1 | −3 | −2 | 2 |
| 106 | K | 0 | 3 | −2 | −3 | −2 | −1 | −1 | −3 | −2 | 3 | 0 | 3 | −1 | −2 | −2 | −1 | −1 | −4 | −2 | 0 |
| 107 | N | −1 | 1 | 3 | 2 | −3 | 2 | 3 | −1 | 0 | −3 | −3 | 0 | −2 | −3 | −2 | 1 | −1 | −4 | −3 | −3 |
| 108 | F | −2 | −3 | −2 | −3 | −2 | −2 | −2 | −3 | 0 | −1 | −1 | −2 | −1 | 6 | −3 | 1 | −2 | 0 | 5 | −2 |
| 109 | S | −1 | 3 | 0 | 0 | −3 | 4 | 3 | −2 | −1 | −3 | −3 | 0 | −1 | −3 | −2 | 2 | −1 | −3 | −2 | −3 |
| 110 | E | −1 | 0 | −2 | −1 | −3 | 0 | 2 | −3 | −2 | 2 | 1 | 3 | 3 | −2 | −2 | −1 | −1 | −3 | −2 | 0 |
| 111 | I | −2 | −1 | −1 | 3 | −3 | 0 | 3 | −3 | −2 | 1 | −2 | 3 | −1 | −3 | −2 | −1 | −1 | −4 | −2 | 0 |
| 112 | A | 5 | −2 | −3 | −3 | 0 | −1 | −1 | −1 | −3 | 0 | −1 | −1 | −1 | −2 | −1 | 0 | 0 | −3 | −2 | 1 |
| 113 | C | 0 | −4 | −4 | −4 | 10 | −4 | −5 | −4 | −4 | −1 | −1 | −4 | −1 | −3 | −4 | −1 | −1 | −3 | −3 | −1 |
| 114 | S | −1 | −1 | 2 | 2 | −3 | 3 | 0 | −1 | −1 | −3 | −3 | 0 | −2 | −4 | 4 | 2 | −1 | −4 | −3 | −3 |
| 115 | E | −1 | 3 | 0 | −1 | −4 | 0 | 3 | −3 | −1 | −4 | −3 | 5 | −2 | −4 | −2 | −1 | −1 | −4 | −3 | −3 |
| 116 | D | −2 | −1 | 0 | 4 | −2 | −1 | 0 | −2 | 5 | −1 | 0 | −1 | −1 | −2 | −2 | 0 | 2 | −3 | −1 | −1 |
| 117 | C | 0 | −4 | −4 | −4 | 10 | −4 | −5 | −4 | −4 | −1 | −1 | −4 | −1 | −3 | −4 | −1 | −1 | −3 | −3 | −1 |
| 118 | I | −1 | 1 | −1 | −2 | −3 | −2 | −2 | 4 | 3 | 2 | −2 | −2 | −2 | −2 | −3 | −1 | −2 | −3 | −2 | −1 |
| 119 | V | −1 | −3 | −3 | −3 | −1 | −2 | −3 | −3 | −3 | 3 | 1 | −2 | 0 | −1 | −3 | 0 | −1 | −3 | −2 | 4 |
| 120 | V | −2 | −3 | −3 | −4 | −2 | −2 | −3 | −4 | −2 | 2 | 1 | −3 | 5 | 4 | −3 | −2 | −2 | −1 | 3 | 1 |
| 121 | E | −1 | 0 | −1 | 0 | −3 | 3 | 3 | −2 | −1 | −2 | −0 | 3 | −1 | −3 | −2 | 1 | −1 | −3 | −2 | −2 |
| 122 | G | 0 | −1 | −1 | −1 | −3 | 3 | 1 | 2 | 0 | −3 | −3 | −1 | −2 | −1 | −2 | −1 | −2 | −1 | 4 | −2 |
| 123 | P | −1 | −1 | −1 | 0 | −3 | 0 | 4 | −3 | −1 | −3 | −3 | 0 | −2 | −4 | 3 | 0 | 3 | −4 | −3 | −2 |
| 124 | I | −1 | −3 | −3 | −3 | −1 | −2 | −3 | −4 | −3 | 2 | 2 | −2 | 0 | −1 | −3 | −2 | 2 | −3 | −2 | 3 |
| 125 | L | −1 | −3 | −4 | −4 | −1 | −3 | −4 | −5 | −4 | 5 | 4 | −3 | 0 | 0 | −4 | −3 | −1 | −3 | −1 | 1 |
| 126 | D | −2 | −3 | 0 | 5 | −3 | −1 | 0 | −1 | −2 | −3 | −3 | −2 | −2 | −2 | −2 | 2 | −1 | 8 | −1 | −3 |
| 127 | C | 0 | −4 | −4 | −4 | 10 | −4 | −5 | −4 | −4 | −1 | −1 | −4 | −1 | −3 | −4 | −1 | −1 | −3 | −3 | −1 |
| 128 | W | −2 | −1 | 1 | −2 | −2 | −1 | −2 | −2 | −2 | 0 | 2 | 1 | 0 | −1 | −3 | 0 | −1 | 8 | −1 | −1 |
| 129 | T | −2 | 0 | 5 | 0 | −3 | 0 | −1 | −1 | 4 | −3 | −3 | 1 | −2 | −1 | −2 | 0 | 1 | −2 | 2 | −3 |
| 130 | C | 0 | −4 | −4 | −4 | 10 | −4 | −5 | −4 | −4 | −1 | −1 | −4 | −1 | −3 | −4 | −1 | −1 | −3 | −3 | −1 |
| 131 | L | −1 | 0 | −1 | −1 | −3 | 2 | 1 | −3 | −2 | −1 | 1 | 3 | 0 | −2 | −2 | −1 | 2 | −3 | −2 | −1 |
| 132 | R | −2 | 4 | 0 | 3 | −4 | 0 | 0 | −2 | −1 | −4 | −3 | 4 | −2 | −4 | −2 | −1 | −1 | −4 | −3 | −3 |
| 133 | M | −1 | −1 | −1 | −1 | −2 | 2 | 1 | −2 | −2 | 1 | 0 | −1 | 4 | −2 | −2 | 2 | 1 | −3 | −2 | 0 |
| 134 | T | −1 | −2 | −1 | −2 | −2 | −1 | −2 | −3 | 5 | 0 | 0 | −2 | 3 | −2 | −2 | −1 | 3 | −3 | −1 | 1 |
| 135 | N | −1 | −2 | 2 | −1 | −3 | −1 | −1 | −2 | 6 | −1 | −2 | −2 | −1 | −2 | 4 | −1 | 1 | −3 | −1 | 1 |
| 136 | R | 2 | 3 | −1 | −2 | −2 | −1 | −1 | −2 | −2 | 2 | −1 | 3 | −1 | −2 | −2 | −1 | −1 | −4 | −2 | 0 |
| 137 | C | 0 | −4 | −4 | −4 | 10 | −4 | −5 | −4 | −4 | −1 | −1 | −4 | −1 | −3 | −4 | −1 | −1 | −3 | −3 | −1 |
| 138 | F | −2 | 3 | −2 | −3 | −2 | −2 | −2 | −3 | −2 | 1 | 0 | −1 | 0 | 5 | −4 | −2 | −2 | −1 | 0 | 1 |
| 139 | K | −1 | 0 | 0 | −1 | −4 | 0 | 0 | 2 | 3 | −4 | −3 | 5 | −2 | −3 | −2 | −1 | −2 | −3 | −2 | −3 |
| 140 | G | 0 | 0 | 0 | −1 | −3 | 0 | 0 | 3 | −2 | −3 | −3 | 3 | −2 | −3 | −2 | 3 | −1 | −3 | −3 | −3 |
| 141 | E | −2 | −1 | −1 | 1 | −3 | 0 | 1 | −3 | 0 | −2 | 0 | 2 | −1 | 0 | −2 | −1 | −2 | −1 | 5 | −2 |
| 142 | Y | −3 | −2 | 1 | 3 | −3 | −1 | −1 | −2 | 0 | −2 | −2 | −2 | −2 | 0 | −3 | −1 | −2 | 0 | 7 | −2 |
| 143 | C | 0 | −4 | −4 | −4 | 10 | −4 | −5 | −4 | −4 | −1 | −1 | −4 | −1 | −3 | −4 | −1 | −1 | −3 | −3 | −1 |

In this case, preferred homologues of the INSP208 polypeptide are defined as those polypeptides which have an E-value of 10⁻² or less when the sequence profile given in Table 2 is input as a query sequence into BLAST version 2.1.3, using the default parameters specified by the NCBI (the National Center for Biotechnology Information; http://www.ncbi.nlm.nih.gov/) [Blosum 62 matrix; gap open penalty=11 and gap extension penalty=1].

TABLE 2

| | | A | R | N | D | C | Q | E | G | H | I | L | K | M | F | P | S | T | W | Y | V |
|---|---|---|---|---|---|---|---|---|---|---|---|---|---|---|---|---|---|---|---|---|---|
| 1 | R | 0 | 2 | −1 | 0 | −3 | 1 | 2 | −3 | −2 | 0 | 0 | 2 | −2 | −3 | −3 | 0 | −2 | −4 | −3 | −2 |
| 2 | D | 0 | 0 | 1 | 2 | −3 | 0 | −1 | 0 | −2 | −1 | 0 | −2 | −1 | −3 | −3 | 0 | 0 | −4 | −3 | 1 |
| 3 | C | −1 | −4 | −4 | −4 | 12 | −4 | −6 | −4 | −4 | −2 | −2 | −4 | −2 | −3 | −4 | −2 | −2 | −3 | −3 | −2 |
| 4 | W | −2 | −2 | −2 | −2 | −3 | 1 | 0 | −3 | 3 | 0 | 1 | 0 | −1 | −2 | 3 | 1 | −2 | 5 | −2 | −2 |
| 5 | I | −2 | −2 | −1 | 0 | −4 | 1 | 0 | 3 | −3 | 0 | −3 | 0 | −2 | −3 | −3 | 0 | 0 | 5 | −3 | 0 |
| 6 | R | −1 | 3 | 2 | 2 | −3 | 0 | 0 | −2 | −1 | −2 | −3 | 1 | −2 | −4 | 1 | 1 | −1 | −4 | −3 | 0 |
| 7 | R | 2 | 1 | −2 | −3 | −2 | −2 | −2 | 3 | −3 | −1 | 0 | −2 | −1 | −3 | −3 | 2 | −1 | −4 | −3 | 1 |
| 8 | F | −2 | −2 | −2 | −3 | −3 | −2 | −2 | −4 | 4 | 0 | 0 | 0 | −1 | 2 | 1 | −2 | 1 | −2 | 3 | 2 |
| 9 | P | −2 | −2 | −2 | −1 | −4 | −1 | 2 | 3 | −3 | −4 | −1 | 0 | −3 | −4 | 5 | −1 | −2 | −4 | −4 | −3 |
| 10 | G | −2 | 3 | 2 | 5 | −4 | −1 | 0 | 0 | −2 | −4 | −4 | 1 | −3 | −4 | −3 | 0 | −2 | −5 | −4 | −4 |
| 11 | M | 0 | −3 | −2 | 1 | −2 | −2 | −2 | −3 | −3 | −1 | −2 | −2 | 2 | 3 | −3 | 1 | 4 | −3 | −1 | 0 |
| 12 | L | −2 | −3 | −3 | −3 | −3 | −3 | 0 | −4 | −3 | 3 | 1 | −3 | 0 | 1 | −4 | 0 | −2 | −3 | 1 | 3 |
| 13 | L | −2 | −3 | −3 | −3 | −3 | 1 | 0 | −4 | −3 | 4 | 2 | −2 | 0 | −1 | −4 | 0 | 0 | −3 | 2 | 0 |
| 14 | D | −2 | 2 | 2 | 4 | −4 | 3 | 0 | 2 | −2 | −4 | −4 | −1 | −3 | −4 | −3 | 0 | −2 | −4 | −4 | −4 |
| 15 | L | 3 | 0 | −2 | −3 | −2 | −2 | −2 | −3 | −2 | 0 | 0 | −2 | −2 | −2 | −3 | 0 | 3 | −3 | 2 | −1 |
| 16 | E | −2 | −2 | −1 | 0 | −4 | 2 | 3 | 4 | −2 | −3 | −4 | 0 | −3 | −4 | −3 | −1 | 0 | −4 | −3 | 0 |
| 17 | E | 0 | −2 | −2 | 2 | −3 | −1 | 4 | −3 | −2 | 0 | 0 | −1 | −2 | −3 | 1 | 0 | −2 | −4 | −3 | −2 |
| 18 | S | 0 | 0 | 0 | 1 | −3 | −1 | 1 | −2 | 3 | −4 | −4 | −1 | −2 | −3 | −2 | 5 | −1 | −4 | −3 | −3 |
| 19 | Q | −2 | 0 | −3 | −3 | −3 | 2 | −2 | 0 | −2 | 2 | 1 | −2 | −1 | −2 | 0 | −2 | 0 | −3 | 2 | 2 |
| 20 | R | 0 | 2 | 3 | −1 | −3 | 0 | 3 | −2 | −1 | −4 | −4 | 2 | −3 | −4 | −2 | 2 | −1 | −5 | −3 | −3 |
| 21 | L | 0 | −2 | 0 | −2 | −3 | 0 | 0 | 1 | −3 | 0 | 2 | 0 | 1 | −3 | 1 | 0 | −2 | −4 | −3 | −1 |
| 22 | G | −1 | −3 | −1 | −1 | −4 | −2 | 1 | 6 | −3 | −4 | −4 | −2 | −3 | 0 | 1 | 0 | −3 | −3 | −3 | −4 |
| 23 | A | 4 | 0 | 1 | −2 | −2 | −2 | −2 | 0 | −3 | 0 | −2 | −2 | −2 | −3 | 1 | 0 | 0 | −4 | −3 | −1 |
| 24 | Q | −1 | 2 | −1 | 1 | −3 | 1 | −1 | 0 | −2 | −3 | −3 | 0 | −2 | −4 | 1 | 0 | 3 | −4 | −3 | 0 |
| 25 | F | 0 | 0 | −2 | −4 | −3 | −3 | −3 | 0 | 5 | 0 | 0 | −3 | −1 | 6 | −4 | −2 | −3 | −1 | 2 | −2 |
| 26 | L | −2 | −3 | 0 | −4 | −2 | −3 | −4 | −5 | −3 | 1 | 5 | −3 | 0 | −1 | −4 | −3 | −2 | −3 | 2 | 1 |
| 27 | K | −2 | 2 | −1 | 2 | −4 | 1 | 2 | 2 | −2 | −3 | −1 | 1 | 2 | −4 | −3 | −1 | 0 | −4 | −3 | −3 |
| 28 | Y | 1 | 0 | −2 | 0 | −3 | −2 | −2 | 0 | −2 | −2 | 0 | −2 | 2 | −2 | 3 | 2 | −1 | −3 | 1 | −2 |
| 29 | Y | 0 | −3 | −3 | −3 | −3 | 1 | −2 | −4 | −3 | 2 | 1 | −2 | −1 | −2 | 5 | −2 | −2 | −3 | 1 | 0 |
| 30 | S | 0 | −1 | −1 | 0 | −3 | 2 | 2 | 0 | 3 | −4 | −4 | 0 | −3 | −4 | 1 | 1 | 1 | −4 | −3 | −3 |
| 31 | E | 0 | 0 | −1 | 1 | −3 | −1 | 2 | 0 | 5 | −1 | 0 | −2 | −1 | −3 | −3 | 0 | −2 | −4 | −2 | 2 |
| 32 | N | 0 | 0 | 2 | 0 | −3 | −1 | 0 | −2 | −2 | −1 | 0 | −2 | −1 | −3 | 0 | 1 | 0 | −4 | −3 | 2 |
| 33 | T | 1 | 1 | −2 | −3 | −2 | −2 | −2 | −2 | −2 | −2 | 0 | 0 | −2 | 1 | −3 | 2 | 3 | −3 | 1 | −2 |
| 34 | G | 0 | 0 | −2 | 0 | −3 | 1 | −2 | 2 | −2 | 0 | −3 | −2 | −3 | −3 | 3 | 0 | 0 | −3 | 2 | −2 |
| 35 | Q | 0 | −2 | −3 | −1 | −3 | 1 | 4 | −3 | −2 | −2 | 0 | −2 | 2 | 1 | −3 | −2 | −2 | 7 | −1 | 0 |
| 36 | K | 1 | −1 | 1 | 5 | −4 | 2 | 2 | −2 | 3 | −4 | −4 | 0 | −3 | −4 | −2 | −1 | −2 | −5 | −3 | −3 |
| 37 | C | −1 | −4 | −4 | −4 | 12 | −4 | −6 | −4 | −4 | −2 | −2 | −4 | −2 | −3 | −4 | −2 | −2 | −3 | −3 | −2 |
| 38 | G | −1 | 0 | −3 | −3 | −3 | 0 | 0 | 0 | −3 | 0 | 1 | 0 | −1 | −3 | −3 | −2 | 0 | −4 | −3 | 4 |
| 39 | R | 3 | 4 | −2 | −3 | −3 | 1 | −1 | 0 | 2 | −3 | −3 | −1 | 1 | −3 | −3 | −1 | 0 | −4 | −3 | −2 |
| 40 | S | 5 | −2 | −2 | −3 | −1 | −2 | −2 | −2 | −3 | −2 | 0 | −2 | −2 | −3 | −2 | 1 | 1 | −4 | −3 | −1 |
| 41 | C | −1 | −4 | −4 | −4 | 12 | −4 | −6 | −4 | −4 | −2 | −2 | −4 | −2 | −3 | −4 | −2 | −2 | −3 | −3 | −2 |
| 42 | C | −1 | −4 | −4 | −4 | 12 | −4 | −6 | −4 | −4 | −2 | −2 | −4 | −2 | −3 | −4 | −2 | −2 | −3 | −3 | −2 |
| 43 | L | −1 | 0 | 1 | 1 | −3 | 0 | 3 | −2 | −2 | −3 | 0 | −1 | −2 | −4 | −2 | 3 | 2 | −4 | −3 | −3 |
| 44 | R | −2 | 3 | −1 | 2 | −4 | 1 | 0 | −3 | −2 | −3 | 0 | 2 | −2 | −4 | −3 | −1 | 3 | −4 | −3 | −3 |
| 45 | K | −1 | 0 | −1 | −1 | −3 | 0 | 0 | −2 | −1 | −3 | −3 | 5 | −1 | −4 | 4 | 0 | −1 | −4 | −3 | −2 |
| 46 | D | −2 | 3 | 0 | 6 | −3 | 0 | 1 | −1 | −1 | −3 | −4 | 0 | −2 | −3 | −1 | 0 | −1 | −4 | −3 | −3 |
| 47 | V | −1 | −2 | 4 | −1 | −2 | −2 | −2 | −2 | −1 | 3 | 0 | −2 | 0 | −1 | −3 | −1 | 0 | −4 | −1 | 3 |
| 48 | S | 2 | −1 | 0 | −1 | −1 | −1 | −1 | 4 | −2 | −2 | −2 | −1 | −2 | −3 | −1 | 2 | 0 | −3 | −3 | −2 |
| 49 | C | −1 | −4 | −4 | −4 | 12 | −4 | −6 | −4 | −4 | −2 | −2 | −4 | −2 | −3 | −4 | −2 | −2 | −3 | −3 | −2 |
| 50 | N | −3 | −2 | 6 | 4 | −4 | −1 | 0 | −2 | 3 | −4 | −4 | −1 | −3 | −4 | −3 | 0 | 0 | −5 | −3 | −4 |
| 51 | V | 1 | −4 | −4 | −5 | −2 | −3 | −3 | −4 | −4 | 1 | 3 | −3 | 0 | −2 | −3 | −2 | −1 | −4 | −2 | 5 |
| 52 | A | 5 | −3 | −4 | −4 | −2 | −2 | −3 | −2 | −3 | −1 | 0 | −2 | 2 | 0 | −3 | −1 | −1 | −3 | 1 | 0 |
| 53 | V | −2 | −4 | −5 | −5 | −2 | −3 | −4 | −5 | −4 | 3 | 4 | −4 | 0 | −1 | −4 | −3 | −2 | 7 | −1 | 3 |
| 54 | F | −2 | −4 | −4 | −5 | −3 | −3 | −4 | −4 | −3 | 1 | 0 | −4 | 4 | 5 | −4 | −3 | −2 | 7 | 0 | 3 |
| 55 | F | −2 | −2 | −2 | 1 | −4 | 2 | 3 | 0 | 3 | −3 | 0 | −1 | −2 | 4 | −3 | −2 | −3 | −2 | 0 | −3 |
| 56 | H | 0 | 0 | 1 | −1 | −4 | 0 | 3 | −3 | 4 | −2 | 1 | 1 | −2 | −3 | −3 | −1 | 0 | −4 | −2 | −2 |
| 57 | D | 0 | 3 | −1 | 1 | −4 | 0 | 0 | 3 | −2 | −4 | −4 | 1 | −3 | −4 | 2 | −1 | −2 | −4 | −4 | −4 |
| 58 | P | 0 | 2 | −1 | −2 | −4 | 2 | 0 | 0 | −2 | −3 | −3 | 2 | 2 | −4 | 2 | 0 | 0 | −4 | −3 | −3 |
| 59 | V | −1 | 0 | −1 | 4 | −3 | −1 | −1 | 3 | −2 | −2 | −3 | −2 | −2 | −4 | 1 | −1 | 1 | −4 | −3 | 0 |
| 60 | H | −1 | −2 | 0 | 3 | −4 | −2 | −1 | 5 | 4 | −4 | −5 | −2 | −4 | −4 | 1 | −1 | −2 | −3 | −2 | −4 |
| 61 | D | 0 | 2 | 0 | 3 | −3 | 0 | 0 | 1 | −2 | −3 | −3 | 1 | −2 | −4 | 2 | 0 | −1 | −4 | −3 | −3 |
| 62 | N | 3 | −1 | 2 | 1 | −2 | 2 | 0 | −1 | −1 | −2 | −2 | −1 | 2 | −3 | −2 | 1 | −1 | −4 | −3 | −1 |
| 63 | V | −1 | 0 | −3 | −3 | −2 | −2 | −2 | −4 | −3 | 2 | 2 | −2 | 0 | −2 | 4 | −2 | −1 | −4 | −2 | 2 |
| 64 | N | 0 | 2 | 6 | 2 | −3 | 0 | 0 | −1 | 0 | −3 | −3 | 0 | −2 | −4 | −2 | 1 | −1 | −5 | −3 | −3 |
| 65 | C | −1 | −4 | −4 | −4 | 12 | −4 | −6 | −4 | −4 | −2 | −2 | −4 | −2 | −3 | −4 | −2 | −2 | −3 | −3 | −2 |
| 66 | L | −3 | −3 | −3 | −4 | −3 | −3 | −3 | −4 | 3 | −1 | 0 | −3 | −1 | 5 | −4 | −3 | −3 | 0 | 7 | 0 |
| 67 | H | −2 | −2 | −2 | −4 | −3 | 1 | −2 | −5 | 6 | 1 | 4 | −3 | 0 | −1 | −4 | −3 | −2 | −3 | −1 | −1 |
| 68 | V | 0 | −4 | −4 | −4 | −2 | −4 | −4 | −4 | −3 | 2 | 0 | −4 | 1 | 6 | −4 | −3 | −2 | −2 | 0 | 3 |
| 69 | H | −2 | 1 | 5 | 1 | −4 | 2 | 0 | −2 | 3 | −4 | −4 | −1 | −2 | 1 | −3 | 0 | 0 | −4 | −2 | −3 |
| 70 | C | −1 | −4 | −4 | −4 | 12 | −4 | −6 | −4 | −4 | −2 | −2 | −4 | −2 | −3 | −4 | −2 | −2 | −3 | −3 | −2 |
| 71 | P | −2 | −2 | −2 | −2 | −3 | 1 | 0 | 1 | −3 | 0 | 0 | −2 | −2 | −3 | 4 | 0 | 1 | 4 | −3 | −2 |
| 72 | T | 0 | −2 | −1 | −2 | −3 | −2 | −2 | −3 | 4 | −3 | 0 | 0 | −2 | −1 | 2 | 2 | 0 | −2 | 4 | −2 |
| 73 | L | −2 | 3 | 1 | −2 | −4 | −1 | 1 | −3 | −2 | −3 | 0 | 2 | −2 | −4 | 3 | 0 | −2 | −4 | −3 | −3 |
| 74 | E | −2 | −1 | 1 | 1 | −5 | 3 | 4 | 2 | −1 | −5 | −4 | 1 | −3 | −4 | −2 | −1 | −2 | −4 | −3 | −4 |
| 75 | S | 1 | 1 | 0 | −3 | −2 | 1 | −2 | −3 | −3 | 1 | −1 | −1 | −1 | −3 | −3 | 1 | −1 | −4 | −3 | 3 |
| 76 | C | −1 | −4 | −4 | −4 | 12 | −4 | −6 | −4 | −4 | −2 | −2 | −4 | −2 | −3 | −4 | −2 | −2 | −3 | −3 | −2 |
| 77 | I | −2 | 2 | −2 | −2 | −4 | 1 | 0 | −3 | 2 | 0 | 0 | 3 | 2 | −3 | 1 | 0 | −2 | −4 | −2 | −2 |
| 78 | L | −3 | −4 | −4 | −4 | −3 | −3 | −3 | −4 | −3 | −1 | 2 | −3 | −1 | 6 | 5 | −3 | −3 | −2 | 0 | −2 |

Typically, greater than 30% identity between two polypeptides is considered to be an indication of functional equivalence. Preferably, functionally equivalent polypeptides of the first aspect of the invention have a degree of sequence identity with the INSP206 or INSP208 polypeptides, or with active fragments thereof, of greater than 80%. More preferred polypeptides have degrees of identity of greater than 85%, 90%, 95%, 98% or 99%, respectively.

The functionally-equivalent polypeptides of the first aspect of the invention may also be polypeptides which have been identified using one or more techniques of structural alignment. For example, the Inpharmatica Genome Threader technology that forms one aspect of the search tools used to generate the Biopendium™ search database may be used (see PCT application WO 01/69507) to identify polypeptides of presently-unknown function which, while having low sequence identity as compared to the INSP206 or INSP208 polypeptides, are predicted to be members of the Cys-rich, cell surface glycoprotein family, by virtue of sharing significant structural homology with the INSP206 or INSP208 polypeptide sequence. By “significant structural homology” is meant that the Inpharmatica Genome Threader predicts two proteins to share structural homology with a certainty of 10% and above.

The polypeptide of the first aspect of the invention also include fragments of the INSP206 and INSP208 polypeptides and fragments of the functional equivalents of the INSP206 and INSP208 polypeptides, provided that those fragments are members of the Cys-rich, cell surface glycoprotein family or have an antigenic determinant in common with the INSP206 or INSP208 polypeptides.

As used herein, the term “fragment” refers to a polypeptide having an amino acid sequence that is the same as part, but not all, of the amino acid sequence of the INSP206 or INSP208 polypeptides or one of their functional equivalents. The fragments should comprise at least n consecutive amino acids from the sequence and, depending on the particular sequence, n preferably is 7 or more (for example, 8, 10, 12, 14, 16, 18, 20 or more). Small fragments may form an antigenic determinant. Fragments according to the invention may be 1-300 amino acids in length, preferably, 5-250, preferably, 7-200, preferably, 10-150, preferably, 15-100, more preferably 20-500 amino acids.

Nucleic acids according to the invention are preferably 10-1100 nucleotides in length, preferably 50-1000 nucleotides, preferably 100-800, preferably 200-750, preferably 300-500 nucleotides in length. Polypeptides according to the invention are preferably 5-350 amino acids in length, preferably 50-300, preferably 100-250, preferably 150-200 amino acids in length.

Fragments of the full length INSP206 or INSP208 polypeptides may consist of individual exons or combinations of neighbouring exon sequences in the polypeptide sequences. The INSP206 polypeptides are predicted to be made up of 7 constituent exons. Accordingly, fragments may include combinations of exon coding sequences made up of exons 1+2, 2+3, 2+3+4 and so on. The INSP208 polypeptides are predicted to be made up of 3 constituent exons. Accordingly, fragments may include combinations of exon coding sequences made up of exons 1+2, 2+3 and 1+3. These exons may be combined with further mature fragments according to the invention. Such fragments are included in the present invention. Fragments may also consist of combinations of different domains of the INSP206 and INSP208 proteins. For example a fragment may consist of combinations of the different extracellular domains of INSP206 and INSP208 as recited above.

Such fragments may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. For instance, certain preferred embodiments relate to a fragment having a pre- and/or pro-polypeptide region fused to the amino terminus of the fragment and/or an additional region fused to the carboxyl terminus of the fragment. However, several fragments may be comprised within a single larger polypeptide.

The polypeptides of the present invention or their immunogenic fragments (comprising at least one antigenic determinant) can be used to generate ligands, such as polyclonal or monoclonal antibodies, that are immunospecific for the polypeptides. Such antibodies may be employed to isolate or to identify clones expressing the polypeptides of the invention or to purify the polypeptides by affinity chromatography. The antibodies may also be employed as diagnostic or therapeutic aids, amongst other applications, as will be apparent to the skilled reader.

The term “immunospecific” means that the antibodies have substantially greater affinity for the polypeptides of the invention than their affinity for other related polypeptides in the prior art. As used herein, the term “antibody” refers to intact molecules as well as to fragments thereof, such as Fab, F(ab′)2 and Fv, which are capable of binding to the antigenic determinant in question. Such antibodies thus bind to the polypeptides of the first aspect of the invention.

By “substantially greater affinity” we mean that there is a measurable increase in the affinity for a polypeptide of the invention as compared with the affinity for known secreted proteins.

Preferably, the affinity is at least 1.5-fold, 2-fold, 5-fold 10-fold, 100-fold, 10³-fold, 10⁴-fold, 10⁵-fold, 10⁶-fold or greater for a polypeptide of the invention than for known secreted proteins such as members of the Cys-rich, cell surface glycoprotein family.

Preferably, there is a measurable increase in the affinity for a polypeptide of the invention as compared with known Cys-rich, cell surface glycoproteins.

Preferably, there is a measurable increase in the affinity for a polypeptide of the invention as compared with natural Cys-rich, cell surface glycoproteins.

If polyclonal antibodies are desired, a selected mammal, such as a mouse, rabbit, goat or horse, may be immunised with a polypeptide of the first aspect of the invention. The polypeptide used to immunise the animal can be derived by recombinant DNA technology or can be synthesized chemically. If desired, the polypeptide can be conjugated to a carrier protein. Commonly used carriers to which the polypeptides may be chemically coupled include bovine serum albumin, thyroglobulin and keyhole limpet haemocyanin. The coupled polypeptide is then used to immunise the animal. Serum from the immunised animal is collected and treated according to known procedures, for example by immunoaffinity chromatography.

Monoclonal antibodies to the polypeptides of the first aspect of the invention can also be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985).

Panels of monoclonal antibodies produced against the polypeptides of the first aspect of the invention can be screened for various properties, i.e., for isotype, epitope, affinity, etc. Monoclonal antibodies are particularly useful in purification of the individual polypeptides against which they are directed. Alternatively, genes encoding the monoclonal antibodies of interest may be isolated from hybridomas, for instance by PCR techniques known in the art, and cloned and expressed in appropriate vectors.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl. Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is, an antibody having two different antigen binding domains, each domain being directed against a different epitope.

Phage display technology may be utilised to select genes which encode antibodies with binding activities towards the polypeptides of the invention either from repertoires of PCR amplified V-genes of lymphocytes from humans screened for possessing the relevant antibodies, or from naive libraries (McCafferty, J. et al., (1990), Nature 348, 552-554; Marks, J. et al., (1992) Biotechnology 10, 779-783). The affinity of these antibodies can also be improved by chain shuffling (Clackson, T. et al., (1991) Nature 352, 624-628).

Antibodies generated by the above techniques, whether polyclonal or monoclonal, have additional utility in that they may be employed as reagents in immunoassays, radioimmunoassays (RIA) or enzyme-linked immunosorbent assays (ELISA). In these applications, the antibodies can be labelled with an analytically-detectable reagent such as a radioisotope, a fluorescent molecule or an enzyme.

Preferred nucleic acid molecules of the second and third aspects of the invention are those which encode a polypeptide sequence as recited in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40 and functionally equivalent polypeptides. These nucleic acid molecules may be used in the methods and applications described herein. The nucleic acid molecules of the invention preferably comprise at least n consecutive nucleotides from the sequences disclosed herein where, depending on the particular sequence, n is 10 or more (for example, 12, 14, 15, 18, 20, 25, 30, 35, 40 or more).

The nucleic acid molecules of the invention also include sequences that are complementary to nucleic acid molecules described above (for example, for antisense or probing purposes).

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones, and peptide nucleic acids (PNA). The term “PNA”, as used herein, refers to an antisense molecule or an anti-gene agent which comprises an oligonucleotide of at least five nucleotides in length linked to a peptide backbone of amino acid residues, which preferably ends in lysine. The terminal lysine confers solubility to the composition. PNAs may be pegylated to extend their lifespan in a cell, where they preferentially bind complementary single stranded DNA and RNA and stop transcript elongation (Nielsen, P. E. et al. (1993) Anticancer Drug Des. 8:53-63).

A nucleic acid molecule which encodes a polypeptide of this invention may be identical to the coding sequence of one or more of the nucleic acid molecules disclosed herein.

These molecules also may have a different sequence which, as a result of the degeneracy of the genetic code, encodes a polypeptide SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 and SEQ ID NO:40. Such nucleic acid molecules may include, but are not limited to, the coding sequence for the mature polypeptide by itself, the coding sequence for the mature polypeptide and additional coding sequences, such as those encoding a leader or secretory sequence, such as a pro-, pre- or prepro-polypeptide sequence; the coding sequence of the mature polypeptide, with or without the aforementioned additional coding sequences, together with further additional, non-coding sequences, including non-coding 5′ and 3′ sequences, such as the transcribed, non-translated sequences that play a role in transcription (including termination signals), ribosome binding and mRNA stability. The nucleic acid molecules may also include additional sequences which encode additional amino acids, such as those which provide additional functionalities.

The nucleic acid molecules of the second and third aspects of the invention may also encode the fragments or the functional equivalents of the polypeptides and fragments of the first aspect of the invention. Such a nucleic acid molecule may be a naturally-occurring variant such as a naturally-occurring allelic variant, or the molecule may be a variant that is not known to occur naturally. Such non-naturally occurring variants of the nucleic acid molecule may be made by mutagenesis techniques, including those applied to nucleic acid molecules, cells or organisms.

Among variants in this regard are variants that differ from the aforementioned nucleic acid molecules by nucleotide substitutions, deletions or insertions. The substitutions, deletions or insertions may involve one or more nucleotides. The variants may be altered in coding or non-coding regions or both. Alterations in the coding regions may produce conservative or non-conservative amino acid substitutions, deletions or insertions.

The nucleic acid molecules of the invention can also be engineered, using methods generally known in the art, for a variety of reasons, including modifying the cloning, processing, and/or expression of the gene product (the polypeptide). DNA shuffling by random fragmentation and PCR reassembly of gene fragments and synthetic oligonucleotides are included as techniques which may be used to engineer the nucleotide sequences. Site-directed mutagenesis may be used to insert new restriction sites, alter glycosylation patterns, change codon preference, produce splice variants, introduce mutations and so forth.

Nucleic acid molecules which encode a polypeptide of the first aspect of the invention may be ligated to a heterologous sequence so that the combined nucleic acid molecule encodes a fusion protein. Such combined nucleic acid molecules are included within the second or third aspects of the invention. For example, to screen peptide libraries for inhibitors of the activity of the polypeptide, it may be useful to express, using such a combined nucleic acid molecule, a fusion protein that can be recognised by a commercially-available antibody. A fusion protein may also be engineered to contain a cleavage site located between the sequence of the polypeptide of the invention and the sequence of a heterologous protein so that the polypeptide may be cleaved and purified away from the heterologous protein.

The nucleic acid molecules of the invention also include antisense molecules that are partially complementary to nucleic acid molecules encoding polypeptides of the present invention and that therefore hybridize to the encoding nucleic acid molecules (hybridization). Such antisense molecules, such as oligonucleotides, can be designed to recognise, specifically bind to and prevent transcription of a target nucleic acid encoding a polypeptide of the invention, as will be known by those of ordinary skill in the art (see, for example, Cohen, J. S., Trends in Pharm. Sci., 10, 435 (1989), Okano, J. Neurochem. 56, 560 (1991); O'Connor, J. Neurochem 56, 560 (1991); Lee et al., Nucleic Acids Res 6, 3073 (1979); Cooney et al., Science 241, 456 (1988); Dervan et al., Science 251, 1360 (1991).

The term “hybridization” as used here refers to the association of two nucleic acid molecules with one another by hydrogen bonding. Typically, one molecule will be fixed to a solid support and the other will be free in solution. Then, the two molecules may be placed in contact with one another under conditions that favour hydrogen bonding. Factors that affect this bonding include: the type and volume of solvent; reaction temperature; time of hybridization; agitation; agents to block the non-specific attachment of the liquid phase molecule to the solid support (Denhardt's reagent or BLOTTO); the concentration of the molecules; use of compounds to increase the rate of association of molecules (dextran sulphate or polyethylene glycol); and the stringency of the washing conditions following hybridization (see Sambrook et al. [supra]).

The inhibition of hybridization of a completely complementary molecule to a target molecule may be examined using a hybridization assay, as known in the art (see, for example, Sambrook et al. [supra]). A substantially homologous molecule will then compete for and inhibit the binding of a completely homologous molecule to the target molecule under various conditions of stringency, as taught in Wahl, G. M. and S. L. Berger (1987; Methods Enzymol. 152:399-407) and Kimmel, A. R. (1987; Methods Enzymol. 152:507-511).

“Stringency” refers to conditions in a hybridization reaction that favour the association of very similar molecules over association of molecules that differ. High stringency hybridisation conditions are defined as overnight incubation at 42° C. in a solution comprising 50% formamide, 5×SSC (150 mM NaCl, 15 mM trisodium citrate), 50 mM sodium phosphate (pH7.6), 5×Denhardts solution, 10% dextran sulphate, and 20 microgram/ml denatured, sheared salmon sperm DNA, followed by washing the filters in 0.1×SSC at approximately 65° C. Low stringency conditions involve the hybridisation reaction being carried out at 35° C. (see Sambrook et al. [supra]). Preferably, the conditions used for hybridization are those of high stringency.

Preferred embodiments of this aspect of the invention are nucleic acid molecules that are at least 70% identical over their entire length to a nucleic acid molecule encoding the INSP206 or INSP208 polypeptides and nucleic acid molecules that are substantially complementary to such nucleic acid molecules. Preferably, a nucleic acid molecule according to this aspect of the invention comprises a region that is at least 80% identical over its entire length to such coding sequences, or is a nucleic acid molecule that is complementary thereto. In this regard, nucleic acid molecules at least 90%, preferably at least 95%, more preferably at least 98%, 99% or more identical over their entire length to the same are particularly preferred. Preferred embodiments in this respect are nucleic acid molecules that encode polypeptides which retain substantially the same biological function or activity as the INSP206 and INSP208 polypeptides.

The invention also provides a process for detecting a nucleic acid molecule of the invention, comprising the steps of: (a) contacting a nucleic probe according to the invention with a biological sample under hybridizing conditions to form duplexes; and (b) detecting any such duplexes that are formed.

As discussed additionally below in connection with assays that may be utilised according to the invention, a nucleic acid molecule as described above may be used as a hybridization probe for RNA, cDNA or genomic DNA, in order to isolate full-length cDNAs and genomic clones encoding the INSP206 and INSP208 polypeptides and to isolate cDNA and genomic clones of homologous or orthologous genes that have a high sequence similarity to the gene encoding these polypeptides.

In this regard, the following techniques, among others known in the art, may be utilised and are discussed below for purposes of illustration. Methods for DNA sequencing and analysis are well known and are generally available in the art and may, indeed, be used to practice many of the embodiments of the invention discussed herein. Such methods may employ such enzymes as the Klenow fragment of DNA polymerase I, Sequenase (US Biochemical Corp, Cleveland, Ohio), Taq polymerase (Perkin Elmer), thermostable T7 polymerase (Amersham, Chicago, Ill.), or combinations of polymerases and proof-reading exonucleases such as those found in the ELONGASE Amplification System marketed by Gibco/BRL (Gaithersburg, Md.). Preferably, the sequencing process may be automated using machines such as the Hamilton Micro Lab 2200 (Hamilton, Reno, Nev.), the Peltier Thermal Cycler (PTC200; MJ Research, Watertown, Mass.) and the ABI Catalyst and 373 and 377 DNA Sequencers (Perkin Elmer).

One method for isolating a nucleic acid molecule encoding a polypeptide with an equivalent function to that of the INSP206 and INSP208 polypeptides is to probe a genomic or cDNA library with a natural or artificially-designed probe using standard procedures that are recognised in the art (see, for example, “Current Protocols in Molecular Biology”, Ausubel et al. (eds). Greene Publishing Association and John Wiley Interscience, New York, 1989, 1992). Probes comprising at least 15, preferably at least 30, and more preferably at least 50, contiguous bases that correspond to, or are complementary to, nucleic acid sequences from the appropriate encoding gene (SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 and SEQ ID NO:39), are particularly useful probes. Such probes may be labelled with an analytically-detectable reagent to facilitate their identification. Useful reagents include, but are not limited to, radioisotopes, fluorescent dyes and enzymes that are capable of catalysing the formation of a detectable product. Using these probes, the ordinarily skilled artisan will be capable of isolating complementary copies of genomic DNA, cDNA or RNA polynucleotides encoding proteins of interest from human, mammalian or other animal sources and screening such sources for related sequences, for example, for additional members of the family, type and/or subtype.

In many cases, isolated cDNA sequences will be incomplete, in that the region encoding the polypeptide will be cut short, normally at the 5′ end. Several methods are available to obtain full length cDNAs, or to extend short cDNAs. Such sequences may be extended utilising a partial nucleotide sequence and employing various methods known in the art to detect upstream sequences such as promoters and regulatory elements. For example, one method which may be employed is based on the method of Rapid Amplification of cDNA Ends (RACE; see, for example, Frohman et al., PNAS USA 85, 8998-9002, 1988). Recent modifications of this technique, exemplified by the Marathon™ technology (Clontech Laboratories Inc.), for example, have significantly simplified the search for longer cDNAs. A slightly different technique, termed “restriction-site” PCR, uses universal primers to retrieve unknown nucleic acid sequence adjacent a known locus (Sarkar, G. (1993) PCR Methods Applic. 2:318-322). Inverse PCR may also be used to amplify or to extend sequences using divergent primers based on a known region (Triglia, T. et al. (1988) Nucleic Acids Res. 16:8186). Another method which may be used is capture PCR which involves PCR amplification of DNA fragments adjacent a known sequence in human and yeast artificial chromosome DNA (Lagerstrom, M. et al. (1991) PCR Methods Applic., 1, 111-119). Another method which may be used to retrieve unknown sequences is that of Parker, J. D. et al. (1991); Nucleic Acids Res. 19:3055-3060). Additionally, one may use PCR, nested primers, and PromoterFinder™ libraries to walk genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences that contain the 5′ regions of genes. Use of a randomly primed library may be especially preferable for situations in which an oligo d(T) library does not yield a full-length cDNA. Genomic libraries may be useful for extension of sequence into 5′ non-transcribed regulatory regions.

In one embodiment of the invention, the nucleic acid molecules of the present invention may be used for chromosome localisation. In this technique, a nucleic acid molecule is specifically targeted to, and can hybridize with, a particular location on an individual human chromosome. The mapping of relevant sequences to chromosomes according to the present invention is an important step in the confirmatory correlation of those sequences with the gene-associated disease. Once a sequence has been mapped to a precise chromosomal location, the physical position of the sequence on the chromosome can be correlated with genetic map data. Such data are found in, for example, V. McKusick, Mendelian Inheritance in Man (available on-line through Johns Hopkins University Welch Medical Library). The relationships between genes and diseases that have been mapped to the same chromosomal region are then identified through linkage analysis (coinheritance of physically adjacent genes). This provides valuable information to investigators searching for disease genes using positional cloning or other gene discovery techniques. Once the disease or syndrome has been crudely localised by genetic linkage to a particular genomic region, any sequences mapping to that area may represent associated or regulatory genes for further investigation. The nucleic acid molecule may also be used to detect differences in the chromosomal location due to translocation, inversion, etc. among normal, carrier, or affected individuals.

The nucleic acid molecules of the present invention are also valuable for tissue localisation. Such techniques allow the determination of expression patterns of the polypeptide in tissues by detection of the mRNAs that encode them. These techniques include in situ hybridization techniques and nucleotide amplification techniques, such as PCR. Results from these studies provide an indication of the normal functions of the polypeptide in the organism. In addition, comparative studies of the normal expression pattern of mRNAs with that of mRNAs encoded by a mutant gene provide valuable insights into the role of mutant polypeptides in disease. Such inappropriate expression may be of a temporal, spatial or quantitative nature.

Gene silencing approaches may also be undertaken to down-regulate endogenous expression of a gene encoding a polypeptide of the invention. RNA interference (RNAi) (Elbashir, S M et al., Nature 2001, 411, 494-498) is one method of sequence specific post-transcriptional gene silencing that may be employed. Short dsRNA oligonucleotides are synthesised in vitro and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The vectors of the present invention comprise nucleic acid molecules of the invention and may be cloning or expression vectors. The host cells of the invention, which may be transformed, transfected or transduced with the vectors of the invention may be prokaryotic or eukaryotic.

The polypeptides of the invention may be prepared in recombinant form by expression of their encoding nucleic acid molecules in vectors contained within a host cell. Such expression methods are well known to those of skill in the art and many are described in detail by Sambrook et al. (supra) and Fernandez & Hoeffler (1998, eds. “Gene expression systems. Using nature for the art of expression”. Academic Press, San Diego, London, Boston, New York, Sydney, Tokyo, Toronto).

Generally, any system or vector that is suitable to maintain, propagate or express nucleic acid molecules to produce a polypeptide in the required host may be used. The appropriate nucleotide sequence may be inserted into an expression system by any of a variety of well-known and routine techniques, such as, for example, those described in Sambrook et al., (supra). Generally, the encoding gene can be placed under the control of a control element such as a promoter, ribosome binding site (for bacterial expression) and, optionally, an operator, so that the DNA sequence encoding the desired polypeptide is transcribed into RNA in the transformed host cell.

Examples of suitable expression systems include, for example, chromosomal, episomal and virus-derived systems, including, for example, vectors derived from: bacterial plasmids, bacteriophage, transposons, yeast episomes, insertion elements, yeast chromosomal elements, viruses such as baculoviruses, papova viruses such as SV40, vaccinia viruses, adenoviruses, fowl pox viruses, pseudorabies viruses and retroviruses, or combinations thereof, such as those derived from plasmid and bacteriophage genetic elements, including cosmids and phagemids. Human artificial chromosomes (HACs) may also be employed to deliver larger fragments of DNA than can be contained and expressed in a plasmid. The vectors pCR4-TOPO-INSP206-PS18-1, pCR4-TOPO-INSP206-PS18-2, pCR4-TOPO-INSP206-PS18-3, pDONR221-INSP206SV3-6HIS, pEAK12d_INSP206SV3-6HIS, pDEST12.2_INSP206SV3-6HIS, pCR4-TOPO-INSP208SV-S117-7, pDONR221, pDONR221-INSP208SV [L112S, T118M]-6HIS, pDON 221_INSP208SV-6HIS, pEAK12d, pDEST12.2, pEAK12d_INSP208SV-6HIS and pDEST12.2_INSP208SV-6HIS, INSP208_BSK. are preferred examples of suitable vectors for use in accordance with the aspects of this invention relating to INSP206 and INSP208.

Particularly suitable expression systems include microorganisms such as bacteria transformed with recombinant bacteriophage, plasmid or cosmid DNA expression vectors; yeast transformed with yeast expression vectors; insect cell systems infected with virus expression vectors (for example, baculovirus); plant cell systems transformed with virus expression vectors (for example, cauliflower mosaic virus, CaMV; tobacco mosaic virus, TMV) or with bacterial expression vectors (for example, Ti or pBR322 plasmids); or animal cell systems. Cell-free translation systems can also be employed to produce the polypeptides of the invention.

Introduction of nucleic acid molecules encoding a polypeptide of the present invention into host cells can be effected by methods described in many standard laboratory manuals, such as Davis et al., Basic Methods in Molecular Biology (1986) and Sambrook et al., (supra). Particularly suitable methods include calcium phosphate transfection, DEAE-dextran mediated transfection, transfection, microinjection, cationic lipid-mediated transfection, electroporation, transduction, scrape loading, ballistic introduction or infection (see Sambrook et al., 1989 [supra]; Ausubel et al., 1991 [supra]; Spector, Goldman & Leinwald, 1998). In eukaryotic cells, expression systems may either be transient (for example, episomal) or permanent (chromosomal integration) according to the needs of the system.

The encoding nucleic acid molecule may or may not include a sequence encoding a control sequence, such as a signal peptide or leader sequence, as desired, for example, for secretion of the translated polypeptide into the lumen of the endoplasmic reticulum, into the periplasmic space or into the extracellular environment. These signals may be endogenous to the polypeptide or they may be heterologous signals. Leader sequences can be removed by the bacterial host in post-translational processing.

In addition to control sequences, it may be desirable to add regulatory sequences that allow for regulation of the expression of the polypeptide relative to the growth of the host cell. Examples of regulatory sequences are those which cause the expression of a gene to be increased or decreased in response to a chemical or physical stimulus, including the presence of a regulatory compound or to various temperature or metabolic conditions. Regulatory sequences are those non-translated regions of the vector, such as enhancers, promoters and 5′ and 3′ untranslated regions. These interact with host cellular proteins to carry out transcription and translation. Such regulatory sequences may vary in their strength and specificity. Depending on the vector system and host utilised, any number of suitable transcription and translation elements, including constitutive and inducible promoters, may be used. For example, when cloning in bacterial systems, inducible promoters such as the hybrid lacZ promoter of the Bluescript phagemid (Stratagene, LaJolla, Calif.) or pSport1™ plasmid (Gibco BRL) and the like may be used. The baculovirus polyhedrin promoter may be used in insect cells. Promoters or enhancers derived from the genomes of plant cells (for example, heat shock, RUBISCO and storage protein genes) or from plant viruses (for example, viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

An expression vector is constructed so that the particular nucleic acid coding sequence is located in the vector with the appropriate regulatory sequences, the positioning and orientation of the coding sequence with respect to the regulatory sequences being such that the coding sequence is transcribed under the “control” of the regulatory sequences, i.e., RNA polymerase which binds to the DNA molecule at the control sequences transcribes the coding sequence. In some cases it may be necessary to modify the sequence so that it may be attached to the control sequences with the appropriate orientation; i.e., to maintain the reading frame.

The control sequences and other regulatory sequences may be ligated to the nucleic acid coding sequence prior to insertion into a vector. Alternatively, the coding sequence can be cloned directly into an expression vector that already contains the control sequences and an appropriate restriction site.

For long-term, high-yield production of a recombinant polypeptide, stable expression is preferred. For example, cell lines which stably express the polypeptide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Mammalian cell lines available as hosts for expression are known in the art and include many immortalised cell lines available from the American Type Culture Collection (ATCC) including, but not limited to, Chinese hamster ovary (CHO), HeLa, baby hamster kidney (BHK), monkey kidney (COS), C127, 3T3, BHK, HEK 293, Bowes melanoma and human hepatocellular carcinoma (for example Hep G2) cells and a number of other cell lines.

In the baculovirus system, the materials for baculovirus/insect cell expression systems are commercially available in kit form from, inter alia, Invitrogen, San Diego Calif. (the “MaxBac” kit). These techniques are generally known to those skilled in the art and are described fully in Summers and Smith, Texas Agricultural Experiment Station Bulletin No. 1555 (1987). Particularly suitable host cells for use in this system include insect cells such as Drosophila S2 and Spodoptera Sf9 cells.

There are many plant cell culture and whole plant genetic expression systems known in the art. Examples of suitable plant cellular genetic expression systems include those described in U.S. Pat. No. 5,693,506; U.S. Pat. No. 5,659,122; and U.S. Pat. No. 5,608,143. Additional examples of genetic expression in plant cell culture has been described by Zenk, Phytochemistry 30, 3861-3863 (1991).

In particular, all plants from which protoplasts can be isolated and cultured to give whole regenerated plants can be utilised, so that whole plants are recovered which contain the transferred gene. Practically all plants can be regenerated from cultured cells or tissues, including but not limited to all major species of sugar cane, sugar beet, cotton, fruit and other trees, legumes and vegetables.

Examples of particularly preferred bacterial host cells include streptococci, staphylococci, E. coli, Streptomyces and Bacillus subtilis cells.

Examples of particularly suitable host cells for fungal expression include yeast cells (for example, S. cerevisiae) and Aspergillus cells.

Any number of selection systems are known in the art that may be used to recover transformed cell lines. Examples include the herpes simplex virus thymidine kinase (Wigler, M. et al. (1977) Cell 11:223-32) and adenine phosphoribosyltransferase (Lowy, I. et al. (1980) Cell 22:817-23) genes that can be employed in tk⁻ or aprt^{±} cells, respectively.

Also, antimetabolite, antibiotic or herbicide resistance can be used as the basis for selection; for example, dihydrofolate reductase (DHFR) that confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al. (1981) J. Mol. Biol. 150:1-14) and als or pat, which confer resistance to chlorsulfuron and phosphinotricin acetyltransferase, respectively. Additional selectable genes have been described, examples of which will be clear to those of skill in the art.

Although the presence or absence of marker gene expression suggests that the gene of interest is also present, its presence and expression may need to be confirmed. For example, if the relevant sequence is inserted within a marker gene sequence, transformed cells containing the appropriate sequences can be identified by the absence of marker gene function. Alternatively, a marker gene can be placed in tandem with a sequence encoding a polypeptide of the invention under the control of a single promoter. Expression of the marker gene in response to induction or selection usually indicates expression of the tandem gene as well.

Alternatively, host cells that contain a nucleic acid sequence encoding a polypeptide of the invention and which express said polypeptide may be identified by a variety of procedures known to those of skill in the art. These procedures include, but are not limited to, DNA-DNA or DNA-RNA hybridizations and protein bioassays, for example, fluorescence activated cell sorting (FACS) or immunoassay techniques (such as the enzyme-linked immunosorbent assay [ELISA] and radioimmunoassay [RIA]), that include membrane, solution, or chip based technologies for the detection and/or quantification of nucleic acid or protein (see Hampton, R. et al. (1990) Serological Methods, a Laboratory Manual, APS Press, St Paul, Minn.) and Maddox, D. E. et al. (1983) J. Exp. Med, 158, 1211-1216).

A wide variety of labels and conjugation techniques are known by those skilled in the art and may be used in various nucleic acid and amino acid assays. Means for producing labelled hybridization or PCR probes for detecting sequences related to nucleic acid molecules encoding polypeptides of the present invention include oligolabelling, nick translation, end-labelling or PCR amplification using a labelled polynucleotide. Alternatively, the sequences encoding the polypeptide of the invention may be cloned into a vector for the production of an mRNA probe. Such vectors are known in the art, are commercially available, and may be used to synthesise RNA probes in vitro by addition of an appropriate RNA polymerase such as T7, T3 or SP6 and labelled nucleotides. These procedures may be conducted using a variety of commercially available kits (Pharmacia & Upjohn, (Kalamazoo, Mich.); Promega (Madison Wis.); and U.S. Biochemical Corp., Cleveland, Ohio)).

Suitable reporter molecules or labels, which may be used for ease of detection, include radionuclides, enzymes and fluorescent, chemiluminescent or chromogenic agents as well as substrates, cofactors, inhibitors, magnetic particles, and the like.

Nucleic acid molecules according to the present invention may also be used to create transgenic animals, particularly rodent animals. Such transgenic animals form a further aspect of the present invention. This may be done locally by modification of somatic cells, or by germ line therapy to incorporate heritable modifications. Such transgenic animals may be particularly useful in the generation of animal models for drug molecules effective as modulators of the polypeptides of the present invention.

The polypeptide can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography is particularly useful for purification. Well known techniques for refolding proteins may be employed to regenerate an active conformation when the polypeptide is denatured during isolation and or purification.

Specialised vector constructions may also be used to facilitate purification of proteins, as desired, by joining sequences encoding the polypeptides of the invention to a nucleotide sequence encoding a polypeptide domain that will facilitate purification of soluble proteins. Examples of such purification-facilitating domains include metal chelating peptides such as histidine-tryptophan modules that allow purification on immobilised metals, protein A domains that allow purification on immobilised immunoglobulin, and the domain utilised in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the polypeptide of the invention may be used to facilitate purification. One such expression vector provides for expression of a fusion protein containing the polypeptide of the invention fused to several histidine residues preceding a thioredoxin or an enterokinase cleavage site. The histidine residues facilitate purification by IMAC (immobilised metal ion affinity chromatography as described in Porath, J. et al. (1992), Prot. Exp. Purif. 3: 263-281) while the thioredoxin or enterokinase cleavage site provides a means for purifying the polypeptide from the fusion protein. A discussion of vectors which contain fusion proteins is provided in Kroll, D. J. et al. (1993; DNA Cell Biol. 12:441-453).

If the polypeptide is to be expressed for use in screening assays, generally it is preferred that it be produced at the surface of the host cell in which it is expressed. In this event, the host cells may be harvested prior to use in the screening assay, for example using techniques such as fluorescence activated cell sorting (FACS) or immunoaffinity techniques. If the polypeptide is secreted into the medium, the medium can be recovered in order to recover and purify the expressed polypeptide. If polypeptide is produced intracellularly, the cells must first be lysed before the polypeptide is recovered.

As indicated above, the present invention also provides novel targets and methods for the screening of drug candidates or leads. These screening methods include binding assays and/or functional assays, and may be performed in vitro, in cell systems or in animals.

In this regard, a particular object of this invention resides in the use of an INSP206 or INSP208 polypeptide as a target for screening candidate drugs for treating or preventing Cys-rich, cell surface glycoprotein related disorders.

Another object of this invention resides in methods of selecting biologically active compounds, said methods comprising contacting a candidate compound with a INSP206 or INSP208 gene or polypeptide, and selecting compounds that bind said gene or polypeptide.

A further other object of this invention resides in methods of selecting biologically active compounds, said method comprising contacting a candidate compound with recombinant host cell expressing a INSP206 or INSP208 polypeptide with a candidate compound, and selecting compounds that bind said INSP206 or INSP208 polypeptide at the surface of said cells and/or that modulate the activity of the INSP206 or INSP208 polypeptide.

A “biologically active” compound denotes any compound having biological activity in a subject, preferably therapeutic activity, more preferably a compound having Cys-rich, cell surface glycoprotein activity, and further preferably a compound that can be used for treating INSP206 or INSP208 related disorders, or as a lead to develop drugs for treating Cys-rich, cell surface glycoprotein related disorders. A “biologically active” compound preferably is a compound that modulates the activity of INSP206 or INSP208.

The above methods may be conducted in vitro, using various devices and conditions, including with immobilized reagents, and may further comprise an additional step of assaying the activity of the selected compounds in a model of Cys-rich, cell surface glycoprotein related disorder, such as an animal model.

Preferred selected compounds are agonists of INSP206 or INSP208, i.e., compounds that can bind to INSP206 or INSP208 and mimic the activity of an endogenous ligand thereof.

A further object of this invention resides in a method of selecting biologically active compounds, said method comprising contacting in vitro a test compound with a INSP206 or INSP208 polypeptide according to the present invention and determining the ability of said test compound to modulate the activity of said INSP206 or INSP208 polypeptide.

A further object of this invention resides in a method of selecting biologically active compounds, said method comprising contacting in vitro a test compound with a INSP206 or INSP208 gene according to the present invention and determining the ability of said test compound to modulate the expression of said INSP206 or INSP208 gene, preferably to stimulate expression thereof.

In another embodiment, this invention relates to a method of screening, selecting or identifying active compounds, particularly compounds active on multiple sclerosis or related disorders, the method comprising contacting a test compound with a recombinant host cell comprising a reporter construct, said reporter construct comprising a reporter gene under the control of a INSP206 or INSP208 gene promoter, and selecting the test compounds that modulate (e.g. stimulate or reduce, preferably stimulate) expression of the reporter gene.

The polypeptide of the invention can be used to screen libraries of compounds in any of a variety of drug screening techniques. Such compounds may activate (agonise) or inhibit (antagonise) the level of expression of the gene or the activity of the polypeptide of the invention and form a further aspect of the present invention. Preferred compounds are effective to alter the expression of a natural gene which encodes a polypeptide of the first aspect of the invention or to regulate the activity of a polypeptide of the first aspect of the invention.

Agonist or antagonist compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Binding to a target gene or polypeptide provides an indication as to the ability of the compound to modulate the activity of said target, and thus to affect a pathway leading to Cys-rich, cell surface glycoprotein related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

Modulation of activity includes, without limitation, stimulation of the surface expression of the INSP206 or INSP208 receptor, modulation of multimerization of said receptor (e.g., the formation of multimeric complexes with other sub-units), etc. The cells used in the assays may be any recombinant cell (i.e., any cell comprising a recombinant nucleic acid encoding an INSP206 or INSP208 polypeptide) or any cell that expresses an endogenous INSP206 or INSP208 polypeptide. Examples of such cells include, without limitation, prokaryotic cells (such as bacteria) and eukaryotic cells (such as yeast cells, mammalian cells, insect cells, plant cells, etc.). Specific examples include E. coli, Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, etc.) as well as primary or established mammalian cell cultures (e.g., produced from fibroblasts, embryonic cells, epithelial cells, nervous cells, adipocytes, etc.).

Compounds that are most likely to be good antagonists are molecules that bind to the polypeptide of the invention without inducing the biological effects of the polypeptide upon binding to it. Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the normal biological activity of the polypeptide is prevented.

The polypeptide of the invention that is employed in such a screening technique may be free in solution, affixed to a solid support, borne on a cell surface or located intracellularly. In general, such screening procedures may involve using appropriate cells or cell membranes that express the polypeptide that are contacted with a test compound to observe binding, or stimulation or inhibition of a functional response. The functional response of the cells contacted with the test compound is then compared with control cells that were not contacted with the test compound. Such an assay may assess whether the test compound results in a signal generated by activation of the polypeptide, using an appropriate detection system. Inhibitors of activation are generally assayed in the presence of a known agonist and the effect on activation by the agonist in the presence of the test compound is observed.

A preferred method for identifying an agonist or antagonist compound of a polypeptide of the present invention comprises:

(a) contacting a cell expressing on the surface thereof the polypeptide according to the first aspect of the invention, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by measuring the level of a signal generated from the interaction of the compound with the polypeptide.

Methods for generating detectable signals in the types of assays described herein will be known to those of skill in the art. A particular example is cotransfecting a construct expressing a polypeptide according to the invention, or a fragment such as the LBD, in fusion with the GAL4 DNA binding domain, into a cell together with a reporter plasmid, an example of which is pFR-Luc (Stratagene Europe, Amsterdam, The Netherlands). This particular plasmid contains a synthetic promoter with five tandem repeats of GAL4 binding sites that control the expression of the luciferase gene. When a potential ligand is added to the cells, it will bind the GAL4-polypeptide fusion and induce transcription of the luciferase gene. The level of the luciferase expression can be monitored by its activity using a luminescence reader (see, for example, Lehman et al. JBC 270, 12953, 1995; Pawar et al. JBC, 277, 39243, 2002).

A further preferred method for identifying an agonist or antagonist of a polypeptide of the invention comprises:

(a) contacting a labelled or unlabeled compound with the polypeptide immobilized on any solid support (for example beads, plates, matrix support, chip) and detection of the compound by measuring the label or the presence of the compound itself; or
(b) contacting a cell expressing on the surface thereof the polypeptide, by means of artificially anchoring it to the cell membrane, or by constructing a chimeric receptor being associated with a second component capable of providing a detectable signal in response to the binding of a compound to the polypeptide, with a compound to be screened under conditions to permit binding to the polypeptide; and
(c) determining whether the compound binds to and activates or inhibits the polypeptide by comparing the level of a signal generated from the interaction of the compound with the polypeptide with the level of a signal in the absence of the compound.

For example, a method such as FRET detection of ligand bound to the polypeptide in the presence of peptide co-activators (Norris et al., Science 285, 744, 1999) might be used.

A further preferred method for identifying an agonist or antagonist of a polypeptide of the invention comprises:

(a) contacting a cell expressing on the surface thereof the polypeptide, the polypeptide being associated with a second component capable of providing a detectable signal in response to the binding of a compound to the polypeptide, with a compound to be screened under conditions to permit binding to the polypeptide; and
(b) determining whether the compound binds to and activates or inhibits the polypeptide by comparing the level of a signal generated from the interaction of the compound with the polypeptide with the level of a signal in the absence of the compound.

In further preferred embodiments, the general methods that are described above may further comprise conducting the identification of agonist or antagonist in the presence of labelled or unlabelled ligand for the polypeptide.

In another embodiment of the method for identifying an agonist or antagonist of a polypeptide of the present invention comprises:

determining the inhibition of binding of a ligand to cells which have a polypeptide of the invention on the surface thereof, or to cell membranes containing such a polypeptide, in the presence of a candidate compound under conditions to permit binding to the polypeptide, and determining the amount of ligand bound to the polypeptide. A compound capable of causing reduction of binding of a ligand is considered to be an agonist or antagonist. Preferably the ligand is labelled.

More particularly, a method of screening for a polypeptide antagonist or agonist compound comprises the steps of:

(a) incubating a labelled ligand with a whole cell expressing a polypeptide according to the invention on the cell surface, or a cell membrane containing a polypeptide of the invention,
(b) measuring the amount of labelled ligand bound to the whole cell or the cell membrane;
(c) adding a candidate compound to a mixture of labelled ligand and the whole cell or the cell membrane of step (a) and allowing the mixture to attain equilibrium;
(d) measuring the amount of labelled ligand bound to the whole cell or the cell membrane after step (c); and
(e) comparing the difference in the labelled ligand bound in step (b) and (d), such that the compound which causes the reduction in binding in step (d) is considered to be an agonist or antagonist.

Similarly, there is provided a method of screening for a polypeptide antagonist or agonist compound which comprises the steps of:

(a) incubating a labelled ligand with a polypeptide according to the invention on any solid support or the cell surface, or a cell membrane containing a polypeptide of the invention.
(b) measuring the amount of labelled ligand bound to the polypeptide on the solid support, whole cell or the cell membrane;
(c) adding a candidate compound to a mixture of labelled ligand and immobilized polypeptide on the solid support, the whole cell or the cell membrane of step (a) and allowing the mixture to attain equilibrium;
(d) measuring the amount of labelled ligand bound to the immobilized polypeptide or the whole cell or the cell membrane after step (c); and
(e) comparing the difference in the labelled ligand bound in step (b) and (d), such that the compound which causes the reduction in binding in step (d) is considered to be an agonist or antagonist.

The INSP206 and INSP208 polypeptides of the present invention may modulate cellular growth and differentiation. Thus, the biological activity of the INSP206 and INSP208 polypeptides can be examined in systems that allow the study of cellular growth and differentiation such as organ culture assays or in colony assay systems in agarose culture. Stimulation or inhibition of cellular proliferation may be measured by a variety of assays.

For example, for observing cell growth inhibition, one can use a solid or liquid medium. In a solid medium, cells undergoing growth inhibition can easily be selected from the subject cell group by comparing the sizes of colonies formed. In a liquid medium, growth inhibition can be screened by measuring culture medium turbity or incorporation of labelled thymidine in DNA. Typically, the incorporation of a nucleoside analog into newly synthesised DNA may be employed to measure proliferation (i.e., active cell growth) in a population of cells. For example, bromodeoxyuridine (BrdU) can be employed as a DNA labelling reagent and anti-BrdU mouse monoclonal antibodies can be employed as a detection reagent. This antibody binds only to cells containing DNA which has incorporated bromodeoxyuridine. A number of detection methods may be used in conjunction with this assay including immunofluorescence, immunohistochemical, ELISA, and colorimetric methods. Kits that include bromodeoxyuridine (BrdU) and anti-BrdU mouse monoclonal antibody are commercially available from Boehringer Mannheim (Indianapolis, Ind.).

The effect of the INSP206 and INSP208 polypeptides upon cellular differentiation can be measured by contacting stem cells or embryonic cells with various amounts of the INSP206 and INSP208 polypeptides and observing the effect upon differentiation of the stem cells or embryonic cells. Tissue-specific antibodies and microscopy may be used to identify the resulting cells.

The INSP206 and INSP208 polypeptides may also be found to modulate immune and/or nervous system cell proliferation and differentiation in a dose-dependent manner in the above-described assays. Thus, the “functional equivalents” of the INSP206 and INSP208 polypeptides include polypeptides that exhibit any of the same growth and differentiation regulating activities in the above-described assays in a dose-dependent manner. Although the degree of dose-dependent activity need not be identical to that of the INSP206 and INSP208 polypeptides, preferably the “functional equivalents” will exhibit substantially similar dose-dependence in a given activity assay compared to the INSP206 and INSP208 polypeptides.

In certain of the embodiments described above, simple binding assays may be used, in which the adherence of a test compound to a surface bearing the polypeptide is detected by means of a label directly or indirectly associated with the test compound or in an assay involving competition with a labelled competitor. In another embodiment, competitive drug screening assays may be used, in which neutralising antibodies that are capable of binding the polypeptide specifically compete with a test compound for binding. In this manner, the antibodies can be used to detect the presence of any test compound that possesses specific binding affinity for the polypeptide.

Assays may also be designed to detect the effect of added test compounds on the production of mRNA encoding the polypeptide in cells. For example, an ELISA may be constructed that measures secreted or cell-associated levels of polypeptide using monoclonal or polyclonal antibodies by standard methods known in the art, and this can be used to search for compounds that may inhibit or enhance the production of the polypeptide from suitably manipulated cells or tissues. The formation of binding complexes between the polypeptide and the compound being tested may then be measured.

Assay methods that are also included within the terms of the present invention are those that involve the use of the genes and polypeptides of the invention in overexpression or ablation assays. Such assays involve the manipulation of levels of these genes/polypeptides in cells and assessment of the impact of this manipulation event on the physiology of the manipulated cells. For example, such experiments reveal details of signaling and metabolic pathways in which the particular genes/polypeptides are implicated, generate information regarding the identities of polypeptides with which the studied polypeptides interact and provide clues as to methods by which related genes and proteins are regulated.

Another technique for drug screening which may be used provides for high throughput screening of compounds having suitable binding affinity to the polypeptide of interest (see International patent application WO84/03564). In this method, large numbers of different small test compounds are synthesised on a solid substrate, which may then be reacted with the polypeptide of the invention and washed. One way of inmobilising the polypeptide is to use non-neutralising antibodies. Bound polypeptide may then be detected using methods that are well known in the art. Purified polypeptide can also be coated directly onto plates for use in the aforementioned drug screening techniques.

The polypeptide of the invention may be used to identify membrane-bound or soluble receptors, through standard receptor binding techniques that are known in the art, such as ligand binding and crosslinking assays in which the polypeptide is labelled with a radioactive isotope, is chemically modified, or is fused to a peptide sequence that facilitates its detection or purification, and incubated with a source of the putative receptor (for example, a composition of cells, cell membranes, cell supernatants, tissue extracts, or bodily fluids). The efficacy of binding may be measured using biophysical techniques such as surface plasmon resonance and spectroscopy. Binding assays may be used for the purification and cloning of the receptor, but may also identify agonists and antagonists of the polypeptide, that compete with the binding of the polypeptide to its receptor. Standard methods for conducting screening assays are well understood in the art.

In another embodiment, this invention relates to the use of a INSP206 or INSP208 polypeptide or fragment thereof, whereby the fragment is preferably a INSP206 or INSP208 gene-specific fragment, for isolating or generating an agonist or stimulator of the INSP206 or INSP208 polypeptide for the treatment of an immune related disorder, wherein said agonist or stimulator is selected from the group consisting of:

1. a specific antibody or fragment thereof including: a) a chimeric, b) a humanized or c) a fully human antibody, as well as;
2. a bispecific or multispecific antibody,
3. a single chain (e.g. scFv) or
4. single domain antibody, or
5. a peptide- or non-peptide mimetic derived from said antibodies or
6. an antibody-mimetic such as a) an anticalin or b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004029224.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

The invention also includes a screening kit useful in the methods for identifying agonists, antagonists, ligands, receptors, substrates, enzymes, that are described above.

The invention includes the agonists, antagonists, ligands, receptors, substrates and enzymes, and other compounds which modulate the activity or antigenicity of the polypeptide of the invention discovered by the methods that are described above.

As mentioned above, it is envisaged that the various moieties of the invention (i.e. the polypeptides of the first aspect of the invention, a nucleic acid molecule of the second or third aspect of the invention, a vector of the fourth aspect of the invention, a host cell of the fifth aspect of the invention, a ligand of the sixth aspect of the invention, a compound of the seventh aspect of the invention) may be useful in the therapy or diagnosis of diseases. To assess the utility of the moieties of the invention for treating or diagnosing a disease one or more of the following assays may be carried out. Note that although some of the following assays refer to the test compound as being a protein/polypeptide, a person skilled in the art will readily be able to adapt the following assays so that the other moieties of the invention may also be used as the “test compound”.

The invention also provides pharmaceutical compositions comprising a polypeptide, nucleic acid, ligand or compound of the invention in combination with a suitable pharmaceutical carrier. These compositions may be suitable as therapeutic or diagnostic reagents, as vaccines, or as other immunogenic compositions, as outlined in detail below.

According to the terminology used herein, a composition containing a polypeptide, nucleic acid, ligand or compound [α] is “substantially free of” impurities [herein, Y] when at least 85% by weight of the total X+Y in the composition is X. Preferably, X comprises at least about 90% by weight of the total of X+Y in the composition, more preferably at least about 95%, 98% or even 99% by weight.

The pharmaceutical compositions should preferably comprise a therapeutically effective amount of the polypeptide, nucleic acid molecule, ligand, or compound of the invention. The term “therapeutically effective amount” as used herein refers to an amount of a therapeutic agent needed to treat, ameliorate, or prevent a targeted disease or condition, or to exhibit a detectable therapeutic or preventative effect. For any compound, the therapeutically effective dose can be estimated initially either in cell culture assays, for example, of neoplastic cells, or in animal models, usually mice, rabbits, dogs, or pigs. The animal model may also be used to determine the appropriate concentration range and route of administration. Such information can then be used to determine useful doses and routes for administration in humans.

The precise effective amount for a human subject will depend upon the severity of the disease state, general health of the subject, age, weight, and gender of the subject, diet, time and frequency of administration, drug combination(s), reaction sensitivities, and tolerance/response to therapy. This amount can be determined by routine experimentation and is within the judgement of the clinician. Generally, an effective dose will be from 0.01 mg/kg to 50 mg/kg, preferably 0.05 mg/kg to 10 mg/kg. Compositions may be administered individually to a patient or may be administered in combination with other agents, drugs or hormones.

A pharmaceutical composition may also contain a pharmaceutically acceptable carrier, for administration of a therapeutic agent. Such carriers include antibodies and other polypeptides, genes and other therapeutic agents such as liposomes, provided that the carrier does not itself induce the production of antibodies harmful to the individual receiving the composition, and which may be administered without undue toxicity. Suitable carriers may be large, slowly metabolised macromolecules such as proteins, polysaccharides, polylactic acids, polyglycolic acids, polymeric amino acids, amino acid copolymers and inactive virus particles.

Pharmaceutically acceptable salts can be used therein, for example, mineral acid salts such as hydrochlorides, hydrobromides, phosphates, sulphates, and the like; and the salts of organic acids such as acetates, propionates, malonates, benzoates, and the like. A thorough discussion of pharmaceutically acceptable carriers is available in Remington's Pharmaceutical Sciences (Mack Pub. Co., N.J. 1991).

Pharmaceutically acceptable carriers in therapeutic compositions may additionally contain liquids such as water, saline, glycerol and ethanol. Additionally, auxiliary substances, such as wetting or emulsifying agents, pH buffering substances, and the like, may be present in such compositions. Such carriers enable the pharmaceutical compositions to be formulated as tablets, pills, dragees, capsules, liquids, gels, syrups, slurries, suspensions, and the like, for ingestion by the patient.

Once formulated, the compositions of the invention can be administered directly to the subject. The subjects to be treated can be animals; in particular, human subjects can be treated.

The pharmaceutical compositions utilised in this invention may be administered by any number of routes including, but not limited to, oral, intravenous, intramuscular, intra-arterial, intramedullary, intrathecal, intraventricular, transdermal or transcutaneous applications (for example, see WO98/20734), subcutaneous, intraperitoneal, intranasal, enteral, topical, sublingual, intravaginal or rectal means. Gene guns or hyposprays may also be used to administer the pharmaceutical compositions of the invention. Typically, the therapeutic compositions may be prepared as injectables, either as liquid solutions or suspensions; solid forms suitable for solution in, or suspension in, liquid vehicles prior to injection may also be prepared.

Direct delivery of the compositions will generally be accomplished by injection, subcutaneously, intraperitoneally, intravenously or intramuscularly, or delivered to the interstitial space of a tissue. The compositions can also be administered into a lesion. Dosage treatment may be a single dose schedule or a multiple dose schedule.

If the activity of the polypeptide of the invention is in excess in a particular disease state, several approaches are available. One approach comprises administering to a subject an inhibitor compound (antagonist) as described above, along with a pharmaceutically acceptable carrier in an amount effective to inhibit the function of the polypeptide, such as by blocking the binding of ligands, substrates, enzymes, receptors, or by inhibiting a second signal, and thereby alleviating the abnormal condition. Preferably, such antagonists are antibodies. Most preferably, such antibodies are chimeric and/or humanised to minimise their immunogenicity, as described previously.

In another approach, soluble forms of the polypeptide that retain binding affinity for the ligand, substrate, enzyme, receptor, in question, may be administered. Typically, the polypeptide may be administered in the form of fragments that retain the relevant portions.

In an alternative approach, expression of the gene encoding the polypeptide can be inhibited using expression blocking techniques, such as the use of antisense nucleic acid molecules (as described above), either internally generated or separately administered. Modifications of gene expression can be obtained by designing complementary sequences or antisense molecules (DNA, RNA, or PNA) to the control, 5′ or regulatory regions (signal sequence, promoters, enhancers and introns) of the gene encoding the polypeptide. Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature (Gee, J. E. et al. (1994) In: Huber, B. E. and B. I. Carr, Molecular and Immunologic Approaches, Futura Publishing Co., Mt. Kisco, N.Y.). The complementary sequence or antisense molecule may also be designed to block translation of mRNA by preventing the transcript from binding to ribosomes. Such oligonucleotides may be administered or may be generated in situ from expression in vivo.

In addition, expression of the polypeptide of the invention may be prevented by using ribozymes specific to its encoding mRNA sequence. Ribozymes are catalytically active RNAs that can be natural or synthetic (see for example Usman, N, et al., Curr. Opin. Struct. Biol (1996) 6(4), 527-33). Synthetic ribozymes can be designed to specifically cleave mRNAs at selected positions thereby preventing translation of the mRNAs into functional polypeptide. Ribozymes may be synthesised with a natural ribose phosphate backbone and natural bases, as normally found in RNA molecules. Alternatively the ribozymes may be synthesised with non-natural backbones, for example, 2′-O-methyl RNA, to provide protection from ribonuclease degradation and may contain modified bases.

RNA molecules may be modified to increase intracellular stability and half-life. Possible modifications include, but are not limited to, the addition of flanking sequences at the 5′ and/or 3′ ends of the molecule or the use of phosphorothioate or 2′ O-methyl rather than phosphodiesterase linkages within the backbone of the molecule. This concept is inherent in the production of PNAs and can be extended in all of these molecules by the inclusion of non-traditional bases such as inosine, queosine and butosine, as well as acetyl-, methyl-, thio- and similarly modified forms of adenine, cytidine, guanine, thymine and uridine which are not as easily recognised by endogenous endonucleases.

For treating abnormal conditions related to an under-expression of the polypeptide of the invention and its activity, several approaches are also available. One approach comprises administering to a subject a therapeutically effective amount of a compound that activates the polypeptide, i.e., an agonist as described above, to alleviate the abnormal condition. Alternatively, a therapeutic amount of the polypeptide in combination with a suitable pharmaceutical carrier may be administered to restore the relevant physiological balance of polypeptide.

Gene therapy may be employed to effect the endogenous production of the polypeptide by the relevant cells in the subject. Gene therapy is used to treat permanently the inappropriate production of the polypeptide by replacing a defective gene with a corrected therapeutic gene.

Gene therapy of the present invention can occur in vivo or ex vivo. Ex vivo gene therapy requires the isolation and purification of patient cells, the introduction of a therapeutic gene and introduction of the genetically altered cells back into the patient. In contrast, in vivo gene therapy does not require isolation and purification of a patient's cells.

The therapeutic gene is typically “packaged” for administration to a patient. Gene delivery vehicles may be non-viral, such as liposomes, or replication-deficient viruses, such as adenovirus as described by Berlner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. For example, a nucleic acid molecule encoding a polypeptide of the invention may be engineered for expression in a replication-defective retroviral vector. This expression construct may then be isolated and introduced into a packaging cell transduced with a retroviral plasmid vector containing RNA encoding the polypeptide, such that the packaging cell now produces infectious viral particles containing the gene of interest. These producer cells may be administered to a subject for engineering cells in vivo and expression of the polypeptide in vivo (see Chapter 20, Gene Therapy and other Molecular Genetic-based Therapeutic Approaches, (and references cited therein) in Human Molecular Genetics (1996), T Strachan and A P Read, BIOS Scientific Publishers Ltd).

Another approach is the administration of “naked DNA” in which the therapeutic gene is directly injected into the bloodstream or muscle tissue.

In situations in which the polypeptides or nucleic acid molecules of the invention are disease-causing agents, the invention provides that they can be used in vaccines to raise antibodies against the disease causing agent.

Vaccines according to the invention may either be prophylactic (i.e. to prevent infection) or therapeutic (i.e. to treat disease after infection). Such vaccines comprise immunising antigen(s), immunogen(s), polypeptide(s), protein(s) or nucleic acid, usually in combination with pharmaceutically-acceptable carriers as described above, which include any carrier that does not itself induce the production of antibodies harmful to the individual receiving the composition. Additionally, these carriers may function as immunostimulating agents (“adjuvants”). Furthermore, the antigen or immunogen may be conjugated to a bacterial toxoid, such as a toxoid from diphtheria, tetanus, cholera, H. pylori, and other pathogens.

Since polypeptides may be broken down in the stomach, vaccines comprising polypeptides are preferably administered parenterally (for instance, subcutaneous, intramuscular, intravenous, or intradermal injection). Formulations suitable for parenteral administration include aqueous and non-aqueous sterile injection solutions which may contain anti-oxidants, buffers, bacteriostats and solutes which render the formulation isotonic with the blood of the recipient, and aqueous and non-aqueous sterile suspensions which may include suspending agents or thickening agents.

The vaccine formulations of the invention may be presented in unit-dose or multi-dose containers. For example, sealed ampoules and vials and may be stored in a freeze-dried condition requiring only the addition of the sterile liquid carrier immediately prior to use. The dosage will depend on the specific activity of the vaccine and can be readily determined by routine experimentation.

Genetic delivery of antibodies that bind to polypeptides according to the invention may also be effected, for example, as described in International patent application WO98/55607.

The technology referred to as jet injection (see, for example, www.powderject.com) may also be useful in the formulation of vaccine compositions.

A number of suitable methods for vaccination and vaccine delivery systems are described in International patent application WO00/29428.

This invention also relates to the use of nucleic acid molecules according to the present invention as diagnostic reagents. Detection of a mutated form of the gene characterised by the nucleic acid molecules of the invention which is associated with a dysfunction will provide a diagnostic tool that can add to, or define, a diagnosis of a disease, or susceptibility to a disease, which results from under-expression, over-expression or altered spatial or temporal expression of the gene. Individuals carrying mutations in the gene may be detected at the DNA level by a variety of techniques.

Nucleic acid molecules for diagnosis may be obtained from a subject's cells, such as from blood, urine, saliva, tissue biopsy or autopsy material. The genomic DNA may be used directly for detection or may be amplified enzymatically by using PCR, ligase chain reaction (LCR), strand displacement amplification (SDA), or other amplification techniques (see Saiki et al., Nature, 324, 163-166 (1986); Bej, et al., Crit. Rev. Biochem. Molec. Biol., 26, 301-334 (1991); Birkenmeyer et al., J. Virol. Meth., 35, 117-126 (1991); Van Brunt, J., Bio/Technology, 8, 291-294 (1990)) prior to analysis.

In one embodiment, this aspect of the invention provides a method of diagnosing a disease in a patient, comprising assessing the level of expression of a natural gene encoding a polypeptide according to the invention and comparing said level of expression to a control level, wherein a level that is different to said control level is indicative of disease. The method may comprise the steps of:

a) contacting a sample of tissue from the patient with a nucleic acid probe under stringent conditions that allow the formation of a hybrid complex between a nucleic acid molecule of the invention and the probe;
b) contacting a control sample with said probe under the same conditions used in step a);
c) and detecting the presence of hybrid complexes in said samples;
wherein detection of levels of the hybrid complex in the patient sample that differ from levels of the hybrid complex in the control sample is indicative of disease.

A further aspect of the invention comprises a diagnostic method comprising the steps of:

a) obtaining a tissue sample from a patient being tested for disease;
b) isolating a nucleic acid molecule according to the invention from said tissue sample; and
c) diagnosing the patient for disease by detecting the presence of a mutation in the nucleic acid molecule which is associated with disease.

To aid the detection of nucleic acid molecules in the above-described methods, an amplification step, for example using PCR, may be included.

Deletions and insertions can be detected by a change in the size of the amplified product in comparison to the normal genotype. Point mutations can be identified by hybridizing amplified DNA to labelled RNA of the invention or alternatively, labelled antisense DNA sequences of the invention. Perfectly-matched sequences can be distinguished from mismatched duplexes by RNase digestion or by assessing differences in melting temperatures. The presence or absence of the mutation in the patient may be detected by contacting DNA with a nucleic acid probe that hybridises to the DNA under stringent conditions to form a hybrid double-stranded molecule, the hybrid double-stranded molecule having an unhybridised portion of the nucleic acid probe strand at any portion corresponding to a mutation associated with disease; and detecting the presence or absence of an unhybridised portion of the probe strand as an indication of the presence or absence of a disease-associated mutation in the corresponding portion of the DNA strand.

Such diagnostics are particularly useful for prenatal and even neonatal testing.

Point mutations and other sequence differences between the reference gene and “mutant” genes can be identified by other well-known techniques, such as direct DNA sequencing or single-strand conformational polymorphism, (see Orita et al., Genomics, 5, 874-879 (1989)). For example, a sequencing primer may be used with double-stranded PCR product or a single-stranded template molecule generated by a modified PCR. The sequence determination is performed by conventional procedures with radiolabelled nucleotides or by automatic sequencing procedures with fluorescent-tags. Cloned DNA segments may also be used as probes to detect specific DNA segments. The sensitivity of this method is greatly enhanced when combined with PCR. Further, point mutations and other sequence variations, such as polymorphisms, can be detected as described above, for example, through the use of allele-specific oligonucleotides for PCR amplification of sequences that differ by single nucleotides.

DNA sequence differences may also be detected by alterations in the electrophoretic mobility of DNA fragments in gels, with or without denaturing agents, or by direct DNA sequencing (for example, Myers et al., Science (1985) 230:1242). Sequence changes at specific locations may also be revealed by nuclease protection assays, such as RNase and S1 protection or the chemical cleavage method (see Cotton et al., Proc. Natl. Acad. Sci. USA (1985) 85: 4397-4401).

In addition to conventional gel electrophoresis and DNA sequencing, mutations such as microdeletions, aneuploidies, translocations, inversions, can also be detected by in situ analysis (see, for example, Keller et al., DNA Probes, 2nd Ed., Stockton Press, New York, N.Y., USA (1993)), that is, DNA or RNA sequences in cells can be analysed for mutations without need for their isolation and/or immobilisation onto a membrane. Fluorescence in situ hybridization (FISH) is presently the most commonly applied method and numerous reviews of FISH have appeared (see, for example, Trachuck et al., Science, 250, 559-562 (1990), and Trask et al., Trends, Genet., 7, 149-154 (1991)).

In another embodiment of the invention, an array of oligonucleotide probes comprising a nucleic acid molecule according to the invention can be constructed to conduct efficient screening of genetic variants, mutations and polymorphisms. Array technology methods are well known and have general applicability and can be used to address a variety of questions in molecular genetics including gene expression, genetic linkage, and genetic variability (see for example: M. Chee et al., Science (1996), Vol 274, pp 610-613).

In one embodiment, the array is prepared and used according to the methods described in PCT application WO95/11995 (Chee et al); Lockhart, D. J. et al. (1996) Nat. Biotech. 14: 1675-1680); and Schena, M. et al. (1996) Proc. Natl. Acad. Sci. 93: 10614-10619). Oligonucleotide pairs may range from two to over one million. The oligomers are synthesized at designated areas on a substrate using a light-directed chemical process. The substrate may be paper, nylon or other type of membrane, filter, chip, glass slide or any other suitable solid support. In another aspect, an oligonucleotide may be synthesized on the surface of the substrate by using a chemical coupling procedure and an ink jet application apparatus, as described in PCT application WO95/25116 (Baldeschweiler et al). In another aspect, a “gridded” array analogous to a dot (or slot) blot may be used to arrange and link cDNA fragments or oligonucleotides to the surface of a substrate using a vacuum system, thermal, UV, mechanical or chemical bonding procedures. An array, such as those described above, may be produced by hand or by using available devices (slot blot or dot blot apparatus), materials (any suitable solid support), and machines (including robotic instruments), and may contain 8, 24, 96, 384, 1536 or 6144 oligonucleotides, or any other number between two and over one million which lends itself to the efficient use of commercially-available instrumentation.

In addition to the methods discussed above, diseases may be diagnosed by methods comprising determining, from a sample derived from a subject, an abnormally decreased or increased level of polypeptide or mRNA. Decreased or increased expression can be measured at the RNA level using any of the methods well known in the art for the quantitation of polynucleotides, such as, for example, nucleic acid amplification, for instance PCR, RT-PCR, RNase protection, Northern blotting and other hybridization methods.

Assay techniques that can be used to determine levels of a polypeptide of the present invention in a sample derived from a host are well-known to those of skill in the art and are discussed in some detail above (including radioimmunoassays, competitive-binding assays, Western Blot analysis and ELISA assays). This aspect of the invention provides a diagnostic method which comprises the steps of: (a) contacting a ligand as described above with a biological sample under conditions suitable for the formation of a ligand-polypeptide complex; and (b) detecting said complex.

Protocols such as ELISA, RIA, and FACS for measuring polypeptide levels may additionally provide a basis for diagnosing altered or abnormal levels of polypeptide expression. Normal or standard values for polypeptide expression are established by combining body fluids or cell extracts taken from normal mammalian subjects, preferably humans, with antibody to the polypeptide under conditions suitable for complex formation The amount of standard complex formation may be quantified by various methods, such as by photometric means.

Antibodies which specifically bind to a polypeptide of the invention may be used for the diagnosis of conditions or diseases characterised by expression of the polypeptide, or in assays to monitor patients being treated with the polypeptides, nucleic acid molecules, ligands and other compounds of the invention. Antibodies useful for diagnostic purposes may be prepared in the same manner as those described above for therapeutics. Diagnostic assays for the polypeptide include methods that utilise the antibody and a label to detect the polypeptide in human body fluids or extracts of cells or tissues. The antibodies may be used with or without modification, and may be labelled by joining them, either covalently or non-covalently, with a reporter molecule. A wide variety of reporter molecules known in the art may be used, several of which are described above.

Quantities of polypeptide expressed in subject, control and disease samples from biopsied tissues are compared with the standard values. Deviation between standard and subject values establishes the parameters for diagnosing disease. Diagnostic assays may be used to distinguish between absence, presence, and excess expression of polypeptide and to monitor regulation of polypeptide levels during therapeutic intervention. Such assays may also be used to evaluate the efficacy of a particular therapeutic treatment regimen in animal studies, in clinical trials or in monitoring the treatment of an individual patient.

A diagnostic kit of the present invention may comprise:

(a) a nucleic acid molecule of the present invention;
(b) a polypeptide of the present invention; or
(c) a ligand of the present invention.

In one aspect of the invention, a diagnostic kit may comprise a first container containing a nucleic acid probe that hybridises under stringent conditions with a nucleic acid molecule according to the invention; a second container containing primers useful for amplifying the nucleic acid molecule; and instructions for using the probe and primers for facilitating the diagnosis of disease. The kit may further comprise a third container holding an agent for digesting unhybridised RNA.

In an alternative aspect of the invention, a diagnostic kit may comprise an array of nucleic acid molecules, at least one of which may be a nucleic acid molecule according to the invention.

To detect polypeptide according to the invention, a diagnostic kit may comprise one or more antibodies that bind to a polypeptide according to the invention; and a reagent useful for the detection of a binding reaction between the antibody and the polypeptide.

Such kits will be of use in diagnosing a disease or susceptibility to disease in which members of the Cys-rich, cell surface glycoprotein family are implicated. Such diseases may include cell proliferative disorders, including neoplasm, melanoma, lung, colorectal, breast, pancreas, head and neck and other solid tumours; myeloproliferative disorders, such as leukemia, non-Hodgkin lymphoma, leukopenia, thrombocytopenia, angiogenesis disorder, Kaposis' sarcoma; autoimmune/inflammatory disorders, including allergy, inflammatory bowel disease, arthritis, psoriasis and respiratory tract inflammation, asthma, and organ transplant rejection; cardiovascular disorders, including hypertension, oedema, angina, atherosclerosis, thrombosis, sepsis, shock, reperfusion injury, and ischemia; neurological disorders including central nervous system disease, Alzheimer's disease, brain injury, amyotrophic lateral sclerosis, and pain; developmental disorders; metabolic disorders including diabetes mellitus, osteoporosis, and obesity, AIDS and renal disease; infections including viral infection, bacterial infection, fungal infection and parasitic infection and other pathological conditions. Preferably, the diseases are those in which lymphocyte antigens are implicated. Such kits may also be used for the detection of reproductive disorders including infertility.

Various aspects and embodiments of the present invention will now be described in more detail by way of example, with particular reference to the INSP206 and INSP208 polypeptides.

It will be appreciated that modification of detail may be made without departing from the scope of the invention.

### BRIEF DESCRIPTION OF THE FIGURES

FIG. 1: INSP206 protein sequence, with the indication of the potential signal peptide cleavage site, the transmembrane domain, the Asn residues predicted to be glycosylation sites, and the relevant cysteine residues.

FIG. 2: Alignment of INSP206 protein sequence with the human genomic DNA encoding this protein (as determined on the basis of the assembly hg16 of July 2003); the predicted transmembrane region domain is boxed; the predicted extracellular region is underlined.

FIG. 3: INSP208 protein sequence, with the indication of the potential signal peptide cleavage site, the transmembrane domain, the Asn residues predicted to be glycosylation sites, and the relevant cysteine residues; the MANSC domain is underlined.

FIG. 4: Alignment of INSP208 protein sequence with the human genomic DNA encoding this protein (as determined on the basis of the assembly hg16 of July 2003); the predicted transmembrane region domain is boxed; the predicted extracellular region is underlined; the predicted MANSC domain is double underlined.

FIG. 5: Alignment of the MANSC domains of INSP208, human HAI1 (NCBI Acc No. NP_{—}857593), human LRP11 (NCBI Acc. No. NP_{—}116221); the numbers in the parenthesis are the numbers of the first and of the last residue of the MANSC domain in each sequence.

FIG. 6: INSP206 cDNA and protein sequence. The position of PCR primers used for cloning is indicated by arrows. The predicted extracellular domain is highlighted in grey

FIG. 7: Nucleotide sequence with translation of the INSP206 PCR product. The position of the point mutation is highlighted in grey.

FIG. 8: Nucleotide sequence with translation of the INSP206 SV1 PCR product

FIG. 9: Nucleotide sequence with translation of the INSP206 SV2 PCR product

FIG. 10: Nucleotide sequence with translation of the INSP206 SV3 PCR product

FIG. 11: Clustal W nucleotide sequence alignment of the predicted INSP206 sequence with the cloned sequences.

FIG. 12: Clustal W amino acid sequence alignment of the predicted INSP206 sequence with the cloned sequences.

FIG. 13: INSP208 cDNA and protein sequence. The position of PCR primers used for cloning is indicated by arrows.

FIG. 14: Nucleotide sequence with translation of the INSP208SV1 PCR product. The positions of the point mutations are highlighted in grey.

FIG. 15: Nucleotide sequence with translation of the INSP208 PCR product. The position of PCR primers used for cloning is indicated by arrows.

FIG. 16: Clustal W nucleotide sequence alignment of the predicted (and cloned) INSP208 sequence with the cloned INSP208SV.

FIG. 17: Clustal W amino acid sequence alignment of the predicted (and cloned) INSP208 sequence with the cloned INSP208SV. The predicted transmembrane domain is highlighted in grey.

TABLE 3

| Amino | | More Preferred |
|---|---|---|
| Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE 4

| Amino | |
|---|---|
| Acid | Synonymous Groups |
| Ser | D-Ser, Thr, D-Thr, allo-Thr, Met, D-Met, Met(O), D-Met(O), |
| | L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or L-1- |
| | oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Aib, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Aib, .beta.-Ala, Acp |
| Ile | D-Ile, Val, D-Val, AdaA, AdaG, Len, D-Leu, Met, D-Met |
| Phe | D-Phe, Tyr, D-Thr, L-Dopa, His, D-His, Trp, D-Trp, |
| | Trans-3,4, or 5-phenylproline, AdaA, AdaG, cis-3,4, or |
| | 5-phenylproline, Bpa, D-Bpa |
| Tyr | D-Tyr, Phe, D-Phe, L-Dopa, His, D-His |
| Cys | D-Cys, S--Me--Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, |
| | Ile, D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S--Me--Cys, Ile, D-Ile, Leu, D-Leu, Val, D-Val |

### EXAMPLES

### Example 1

### Identification and in Silico Analysis of INSP206

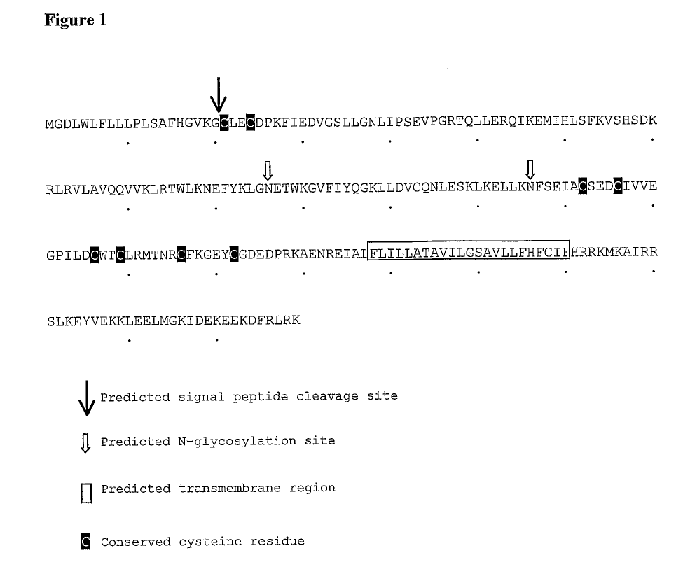

The INSP206 polypeptide sequence contains 239 amino acids (SEQ ID NO: 2). INSP206 is a Cys-rich, cell surface glycoprotein including a signal peptide (residues 1-20), a series of relevant Cysteines (residues 21, 24, 132, 136, 146, 149, 156, and 162) and potential N-glycosylation sites (residues 96 and 126) localized in the N-terminal region predicted as being the extracellular region of the protein (residues 1-177). The transmembrane region (residues 178-200) and intracellular region (residues 201-239) complete the protein sequence (FIG. 1).

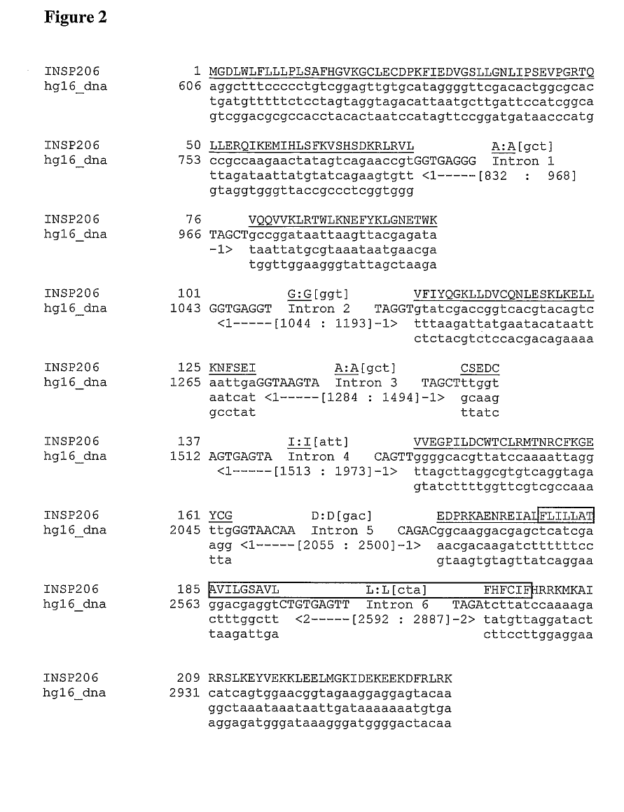

The INSP206 polypeptide sequence results from the transcription and translation of seven coding exons, the first five of them encoding most of the extracellular region (FIG. 2).

When INSP206 is used as a BLAST query against multiple sequence databases including the Genbank non-redundant sequence database and the Derwent GENESEQ database, the majority of top hits (homologous either at the level of specific segments or of the whole sequence) are transmembrane proteins. These sequences, mostly unannotated (for example SEQ ID NO: 38099 in the PCT application WO02/68579, SEQ ID NO: 14 in the PCT application WO03/16506, and SEQ ID NO: 2606 in the PCT application WO01/53312) contain some Cysteines that appear amongst the most conserved amino acids, then predicted as being potentially the more relevant residues.

### Example 2

### Identification and in Silico Analysis of INSP208

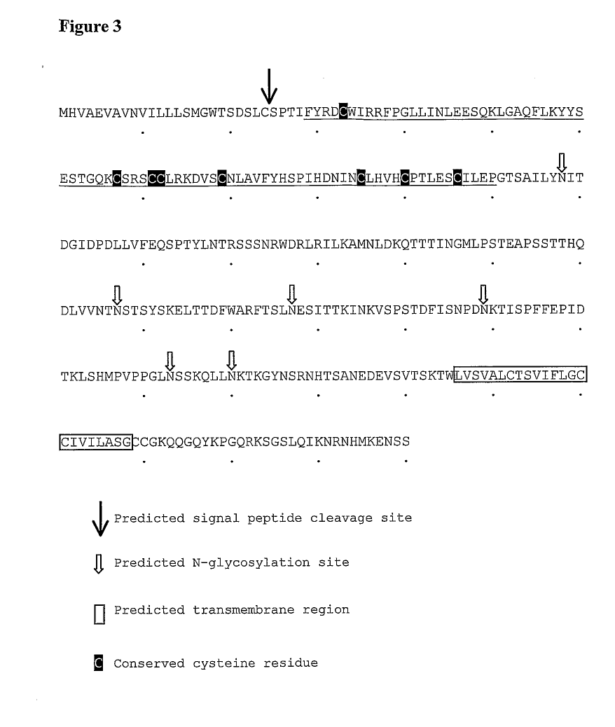

The INSP208 polypeptide sequence contains 340 amino acids (SEQ ID NO: 12). INSP208 is a Cys-rich, cell surface glycoprotein including a signal peptide (residues 1-24), a series of relevant Cysteines (residues 33, 67, 71, 72, 79, 95, 100, and 106), a series of potential N-glycosylation sites (residues 118, 187, 207, 229, 253 and 260), and a MANSC domain (Guo et al., Trends Biochem. Sci. 2004 29(4):172-4) localized in the N-terminal region predicted as being the extracellular region of the protein (residues 1-285). The transmembrane region (residues 286-308) and intracellular region (residues 309-340) complete the protein sequence (FIG. 3).

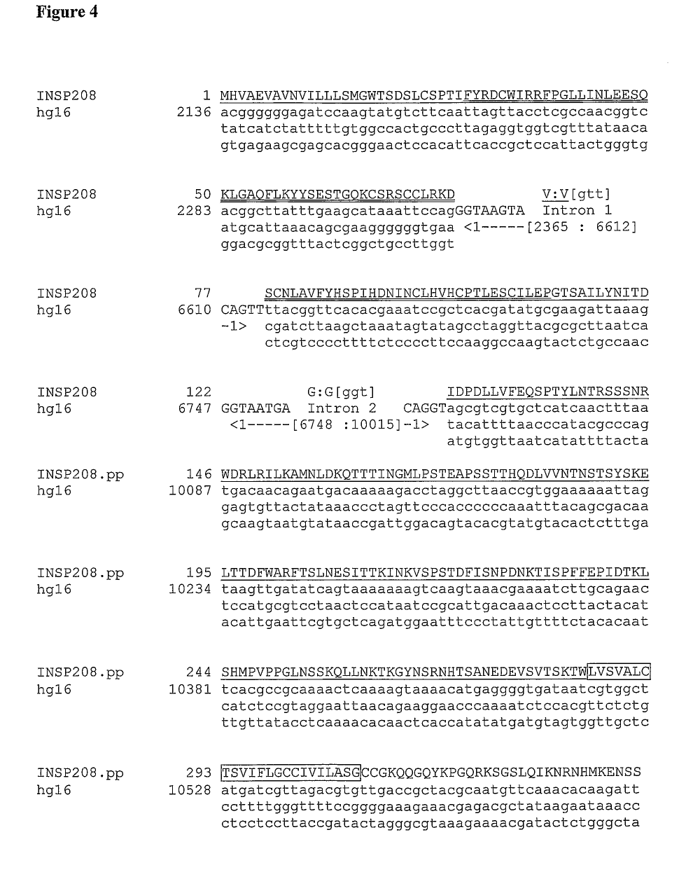

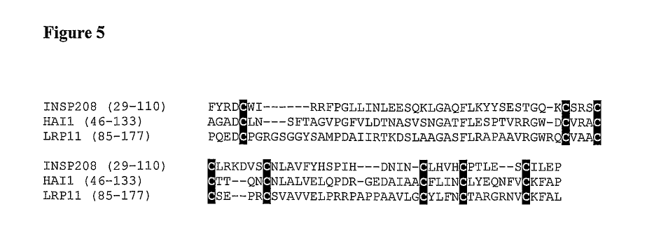

The INSP208 polypeptide sequence results from the transcription and translation of three coding exons, the first two of them encoding the totality of the MANSC domain (FIG. 4). When INSP208 is used as a BLAST query against multiple sequence databases including the Genbank non-redundant sequence database and the Derwent GENESEQ database, the top hits (homologous either at the level of specific segments or of the whole sequence) are all secreted proteins containing the Cys-rich MANSC domain (Guo et al., Trends Biochem. Sci. 2004 29(4):172-4), which is present at the N-terminus of membrane and extracellular proteins, including lipoprotein receptor-related protein 11 (LRP-11), hepatocyte growth factor activator inhibitor 1 (HAI-1), and a number of uncharacterised proteins (FIG. 5).

In particular, HAI-1 has been shown to inhibit proteases involved in the proteolytic conversion and maturation of precursor proteins into molecules stimulating tumour cell-cell interactions, matrix adhesion, migration, invasion and angiogenesis (Kirchhofer D. et al. 2003 J Biol Chem, 278(38):36341-9 & Denda K et al. 2002 J Biol Chem, 277(16):14053-9). LRP11 (also known as LR11 or sorLA) is synthesised as a proreceptor that is cleaved by furin in late Golgi compartments and its proteolytic activation can allow the binding of multiple ligands (Jacobsen L et al. 2001 J Biol Chem 276(25):22788-96). Furthermore, LRP11 has been shown to have a potential role as a mediator of cellular drug uptake (Chung N S & Wasan K M 2004, Adv Drug Deliv Rev 56(9):1315-34) and has also been shown to be associated with Alzheimer's disease (Scherzer et al. 2004 Arch Neurol 61(8):1200-5), neuropeptide head activator signalling and function (Lintzel J et al. 2002 Biol Chem 383(11):1727-33) and atherosclerosis (Bujo H & Saito Y, 2000 J Atheroscler Thromb 7(1):21-5).

Therefore INSP208, being MANSC domain likely to be involved in regeneration of tissue following injury, Alzheimer's disease, atherosclerosis, cancer, and metastasis, may be involved in these pathologies, as a cell-bound receptor or as a soluble protein.

### Example 3

### Cloning of INSP206, -SV1, -SV2 and SV3

### 3.1 Gene Specific Cloning Primers for PCR

PCR primers were designed for amplifying the predicted coding sequence of the virtual cDNA using Primer Designer Software (Scientific & Educational Software, PO Box 72045, Durham, N.C. 27722-2045, USA). Primers were selected which had high selectivity for the target sequence (INSP206).

3.2 Preparation of Human cDNA Templates

First strand cDNA was prepared from a variety of human tissue total RNA samples (Clontech, Stratagene, Ambion, Biochain Institute and in-house preparations) using SuperScript II or SuperScript III RNase H—Reverse Transcriptase (Invitrogen) according to the manufacturer's protocol.

For SuperScript II: Oligo (dT)₁₅ primer (1 μl at 500 μg/ml) (Promega), 2 μg human total RNA, 1 μl 10 mM dNTP mix (10 mM each of DATP, dGTP, dCTP and dTTP at neutral pH) and sterile distilled water to a final volume of 12 μl were combined in a 1.5 ml Eppendorf tube, heated to 65° C. for 5 min and chilled on ice. The contents were collected by brief centrfigation and 4 μl of 5× First-Strand Buffer, 2 μl 0.1 M DTT, and 1 μl RnaseOUT™ Recombinant Ribonuclease Inhibitor (40 units/l, Invitrogen) were added. The contents of the tube were mixed gently and incubated at 42° C. for 2 min, then 1 μl (200 units) of SuperScript II™ enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min.

For SuperScript III: 1 μl Oligo(dT)₂₀ primer (50 μM, Invitrogen), 2 μg human total RNA, 1 μl 10 mM dNTP mix (10 mM each of dATP, dGTP, dCTP and dTTP at neutral pH) and sterile distilled water to a final volume of 10 μl were combined in a 1.5 ml Eppendorf tube, heated to 65° C. for 5 min and then chilled on ice. For each RT reaction a cDNA synthesis mix was prepared as follows: 2 μl 10×RT buffer, 4 μl 25 mM MgCl₂, 2 μl 0.1M DTT, 1 μl RNaseOUT™ (40 U/μl) and 1 μl SuperScript III™ RT enzyme were combined in a separate tube and then 10 μl of this mix added to the tube containing the RNA/primer mixture. The contents of the tube were mixed gently, collected by brief centrifugation, and incubated at 50° C. for 50 min. The reaction was terminated by incubating at 80° C. for 5 min and the reaction mixture then chilled on ice and collected by brief centrifugation. To remove RNA complementary to the cDNA, 1 μl (2 units) of E. coli RNase H (Invitrogen) was added and the reaction mixture incubated at 37° C. for 20 min.

The final 21 μl reaction mix was diluted by adding 179 μL sterile water to give a total volume of 200 μl. This represented approximately 20 ng/μl of each individual cDNA template.

3.3 PCR Amplification of INSP206 from Human cDNA Templates

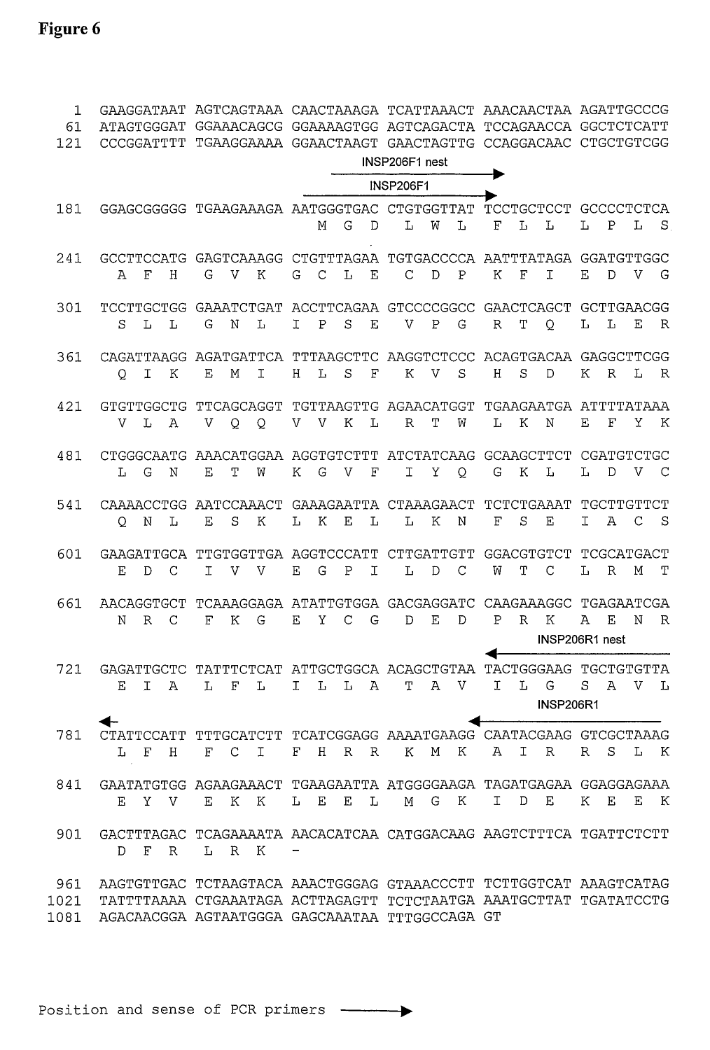

Gene-specific cloning primers (INSP206-CP1 and INSP206-CP2, Table 5, and FIG. 6) were designed to amplify a cDNA fragment of 683 bp containing the extracellular and transmembrane domain of the INSP206 cds. The primer pair was therefore used to screen the collection of human cDNA templates. PCR reactions were performed in a final volume of 50 μl containing 1× Amplitaq™ buffer, 200 μM dNTPs, 1 μM of each cloning primer, 2.5 units of Amplitaq™, and 5 μl of each cDNA template or 1 μl of each cDNA library. Cycling was performed using an MJ Research DNA Engine, programmed as follows: 94° C., 1 min; 40 cycles of 94° C., 1 min, 51° C., 30 sec, and 72° C., 1 min; followed by 1 cycle at 72° C. for 7 min and a holding cycle at 4° C.

1 μl of each PCR reaction was then subjected to a second PCR using nested primers (INSP206 F1 nest and INSP206 R1 nest, FIG. 6 and Table 5) in a final volume of 50 μl containing 1× Amplitaq™ buffer, 200 μM dNTPs, 1 μM of each cloning primer, 2.5 units of Amplitaq™. Cycling was performed using an MJ Research DNA Engine, programmed as follows: 94° C., 1 min; 40 cycles of 94° C., 1 min, 48° C., 1 min, and 72° C., 1 min; followed by 1 cycle at 72° C. for 7 min and a holding cycle at 4° C.

PCR products (40 μl) were visualized on a 0.8% agarose gel in 1×TAE buffer (Invitrogen).

Products of approximately the expected molecular weight (639 bp from PCR1 and 577 bp from PCR 2) were purified from the gel using the Wizard PCR Preps DNA Purification System (Promega), eluted in 50 μl of nuclease free water and subcloned directly.

### 3.4 Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

### 3.5 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Applied Biosystems) using an MJ Research DNA Engine. The cycling conditions were as follows: 94° C., 2 min; 30 cycles of 94° C., 30 sec, 47° C., 30 sec and 72° C. for 1 min. Samples were maintained at 4° C. (holding cycle) before further analysis.

PCR products were analyzed on 1% agarose gels in 1×TAE buffer. Colonies which gave PCR products of approximately the expected molecular weight (639 bp+187 bp due to the multiple cloning site (MCS) were grown up overnight at 37° C. in 5 ml L-Broth (LB) containing ampicillin (100 μg/ml), with shaking at 220 rpm.

### 3.6 Plasmid DNA Preparation and Sequencing

Colonies which gave the expected band size by colony PCR were inoculated into 5 ml L-Broth (LB) containing ampicillin (100 μg/ml) and grown overnight at 37° C. with shaking at 220 rpm. Miniprep plasmid DNA was prepared from the 5 ml culture using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Plasmid DNA (200-500 ng) was subjected to DNA sequencing with the T7 and T3 primers using the BigDye Terminator system (Applied Biosystems cat. no. 4390246) according to the manufacturer's instructions. The primer sequences are shown in Table 5. Sequencing reactions were purified using Dye-Ex columns (Qiagen) or Montage SEQ 96 cleanup plates (Millipore cat. no. LSKS09624) then analyzed on an Applied Biosystems 3700 sequencer.

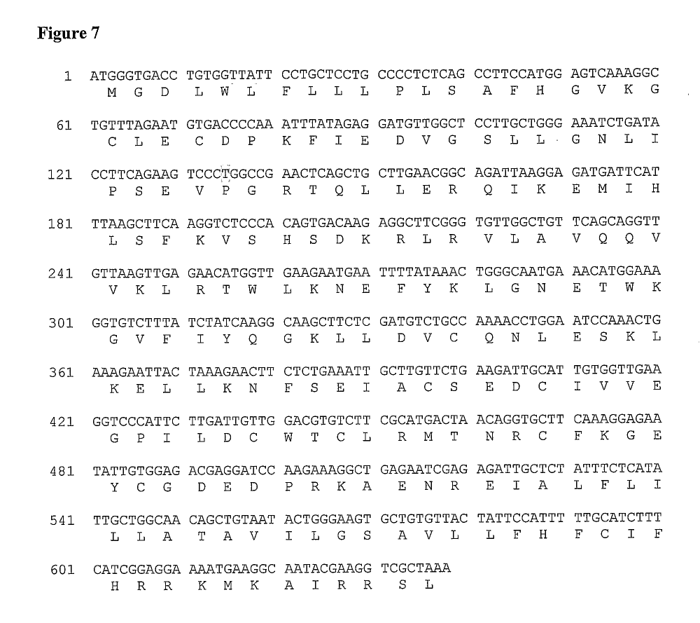

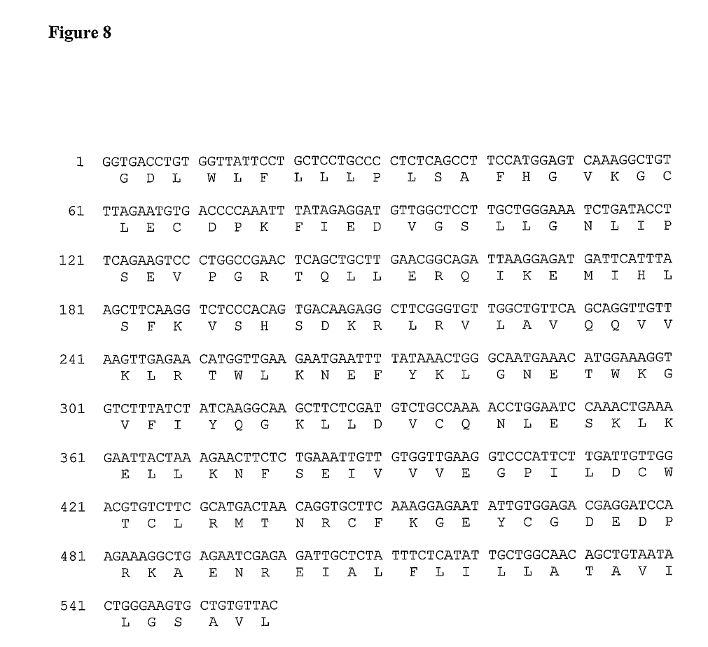

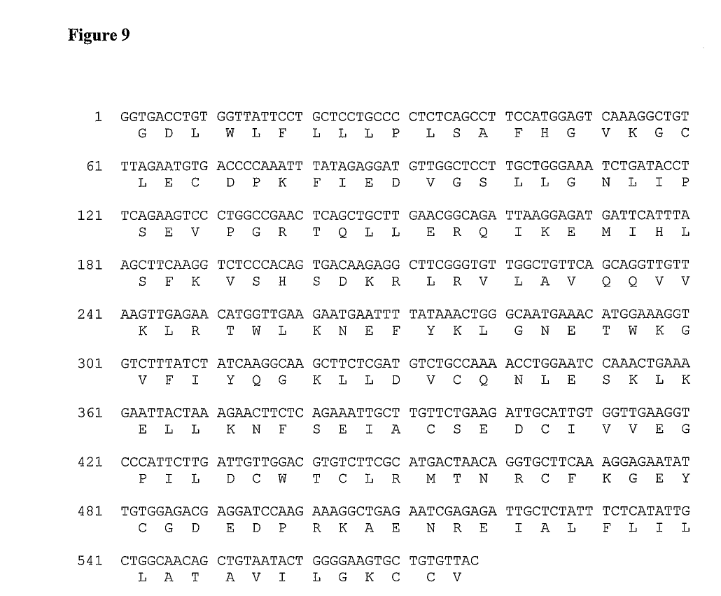

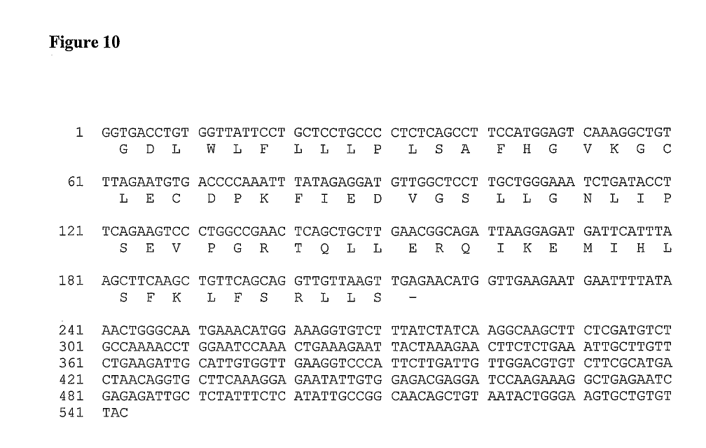

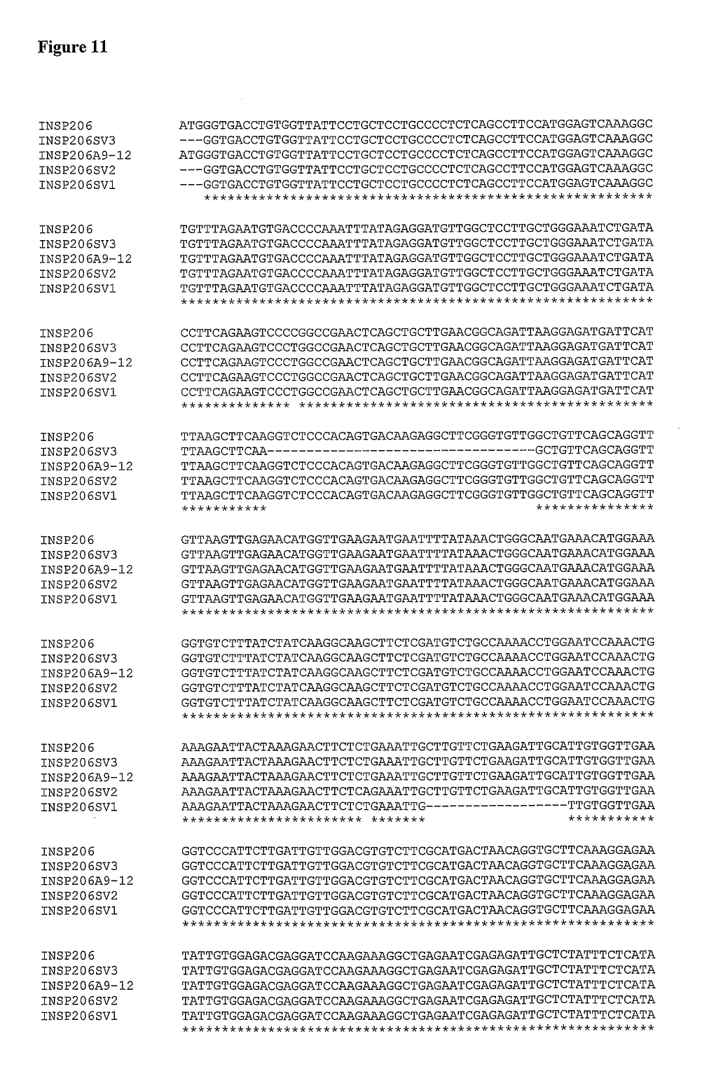

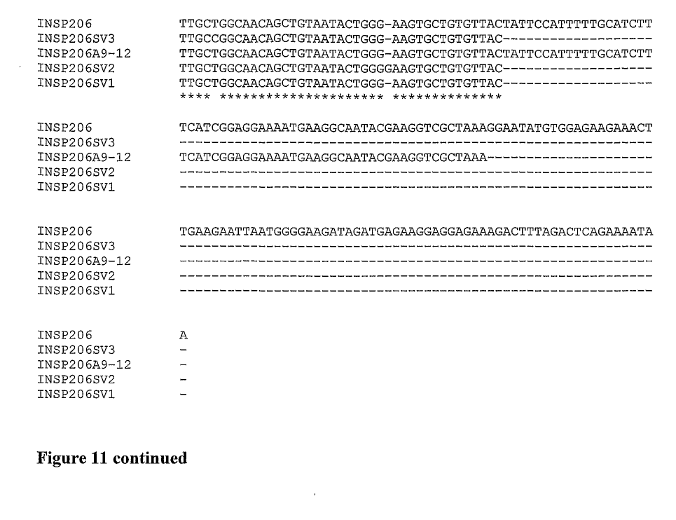

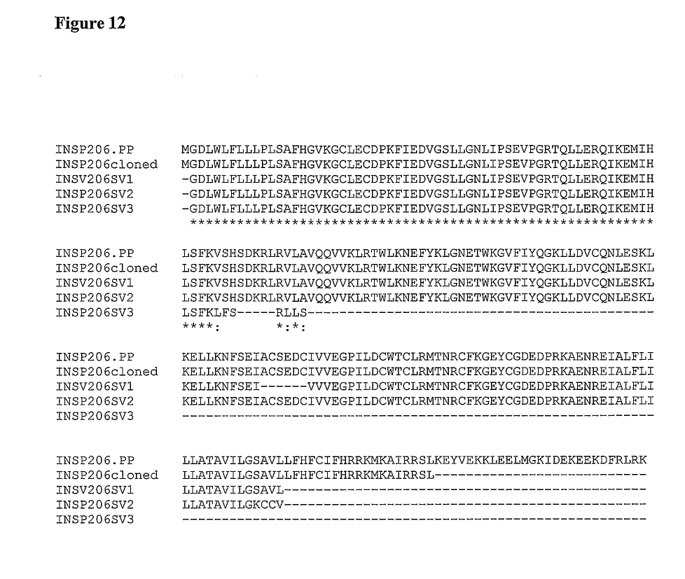

PCR products corresponding to the predicted INSP206 sequence were identified in 4 clones analysed from pool PS18 (a cDNA pool derived from colon, ovary, prostate, testis and normal adult skin cDNAs) amplified using INSP206 F1/R1. The sequence was identical to the original prediction except for a single point mutation in the position corresponding to nucleotide 336 of the original prediction (FIG. 7). This may represent a polymorphic variant. After the nested PCR, 3 possible splice variants were also identified in PS18: PS-18-1 is a splice variant INSP206SV1, which contains an 18 bp deletion (exon 4). The sequence of the cloned cDNA fragment is shown in FIG. 8. The plasmid containing the cloned PCR product is pCR4-TOPO-INSP206-PS18-1. PS-18-2 (INSP206SV2) is a variant of INSP206 which is identical to the predicted INSP206 sequence over the region amplified except it contains a single nucleotide insertion (G) at the 3′ end of the cloned sequence leading to a frame shift. The result of the frameshift is that the sequence of the last 4 amino acids shifts from SAVL to KCCV. The sequence of the cloned cDNA fragment is shown in FIG. 9. The plasmid containing the cloned PCR product is pCR4-TOPO-INSP206-PS18-2. PS-18-3 (INSP206SV3) is a truncated version of INSP206 which contains a 34 bp deletion in exon 1 leading to a frameshift and premature termination. The sequence of the cloned cDNA fragment is shown in FIG. 10. The plasmid containing the cloned PCR product is pCR4-TOPO-INSP206-PS18-3.

### 3.7 Construction of Mammalian Cell Expression Vectors for INSP206-SV3

Plasmid pCR4-TOPO-INSP206-PS18-3 was used as PCR template to generate pEAK12d and pDEST12.2 expression clones containing the INSP206-SV3 ORF sequence with a 3′ sequence encoding a 6HIS tag using the Gateway™ cloning methodology (Invitrogen).

INSP206SV3 was cloned by nested PCR and therefore the cDNA insert in the pCR4-TOPO clone (plasmid pCR4-TOPO-INSP206-PS18-3) was missing the start codon at the 5′ end of the coding sequence. Incorporation of start codon (ATG), kozak sequence (GCC ACC), C-terminal 6HIS tag and stop codon were all accomplished by including the appropriate nucleotides in the primers used for PCR amplification.

### Example 4

### Gateway Cloning

### 4.1 Generation of Gateway Compatible INSP206SV3 ORF Fused to an in Frame 6HIS Tag Sequence.

Plasmid pCR4-TOPO-INSP206-PS18-3 was used as PCR template to generate the full-length ORF containing a C-terminal 6HIS tag and a stop codon. The first stage of this Gateway cloning process involved a two step PCR reaction which generates the full-length ORF of INSP206SV3 flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction PCR1, (in a final volume of 50 μl) contains respectively: 1 μl (25 ng) of plasmid pCR4-TOPO-INSP206-PS18-3, 4.0 μl dNTPs (10 mM), 5 μl of 10× Pfx polymerase buffer, 1.5 μl MgSO4 (50 mM), 1.0 μl each of gene specific primer (to give a final concentration of 100 pico-moles) (INSP206 attB FP and INSP206SV3 attB RP), and 0.5 μl Platinum Pfx DNA polymerase (Invitrogen). The PCR reaction was performed using an initial denaturing step of 95° C. for 2 min, followed by 30 cycles of 94° C. for 30 s; 60° C. for 30 s and 68° C. for 30 s; and a final extension cycle of 68° C. for 5 minutes and a holding cycle of 4° C. The amplification product was directly purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (213 bp+30 bp=243 bp)

The second PCR reaction (in a final volume of 50 μl) contained 1 μl of diluted purified PCR1 product (to a final concentration of 10 ng), 4.0 μl dNTPs (10 mM), 5 μl of 10× Pfx polymerase buffer, 1.5 μl MgSO₄ (50 mM), 1.0 μl of each Gateway conversion primer (to give a final concentration of 100 picomoles) (ATTB PCR FP [unique] and ATTB PCR RP [unique]) and 0.5 μl of Platinum Pfx DNA polymerase. The conditions for the 2nd PCR reaction were: 95° C. for 2 min, followed by 30 cycles of 94° C. for 30 s; 60° C. for 30 s and 68° C. for 30 s; and a final extension cycle of 68° C. for 5 minutes and a holding cycle of 4° C. The PCR gel purified using the Perfectprep Gel cleanup kit (Eppendorf) and recovered in 50 μl sterile water according to the manufacturer's instructions. A 2 μl aliquot was visualized on 1.6% agarose gel in 1×TAE buffer in order to verify that the product was of the expected molecular weight (243 bp+58 bp=301 bp)

4.2 Subcloning of Gateway Compatible INSP206SV3-6HIS ORF into Gateway Entry Vector pDONR221

The second stage of the Gateway cloning process involved sub cloning of the Gateway modified PCR product into the Gateway entry vector pDONR221 (Invitrogen) as follows: 5 μl of gel extracted product from PCR2 was incubated with 1.5 μl pDONR21 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 45 s. Samples were returned to ice and 250 μl of warm SOC media (room temperature) was added. Samples were incubated with shaking (250 rpm) for 1 h at 37° C. The transformation mixture was then plated on L-broth (LB) plates containing kanamycin (50 μg/ml) and incubated overnight at 37° C.

Five numbers of transformants were picked and patched on LB agar plates containing kanamycin (50 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 picomoles) (21M13 FP and ATTB1 PCR RP [unique]) and 0.5 μl of Taq DNA polymerase.

The conditions for the screening PCR reaction were: 95° C. for 2 min, followed by 30 cycles of 94° C. for 30 s; 60° C. for 30 s and 72° C. for 45 s; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from 5 ml cultures using QIAprep Spin Miniprep kit (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 5. Sequencing reactions were analyzed on CEQ 2000 XL DNA analysis system (Beckman Coulter P/N 608450). After sequence confirmation of the insert, pDONR221_INSP206SV3-6HIS, was then used for creating the expression clones.

4.3 Subcloning of Gateway Compatible INSP206SV3 ORF into Expression Vectors pEAK12d and pDEST12.2

Plasmid DNA (2 μl or approx. 150 ng) of pDONR221-INSP206SV3-6HIS was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl.

The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 45 s. Samples were returned to ice and 250 μl of warm SOC media (room temperature) was added. Samples were incubated with shaking (250 rpm) for 1 h at 37° C. The transformation mixture was then plated on L-broth (LB) plates containing Ampicillin (100 μg/ml) and incubated overnight at 37° C.

Five numbers of transformants were picked and patched on LB agar plates containing Ampicillin (100 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 picomoles and 0.5 μl of Taq DNA polymerase. pEAK12d clones were screened using the primers pEAK12 FP and pEAK12 RP and pDEST12.2 clones were screened using the primers 21M13FP and M13Rev RP.

The conditions for the screening PCR reaction were: 95° C. for 2 min, followed by 30 cycles of 94° C. for 30 s; 60° C. for 30 s and 72° C. for 45 s; and a final extension cycle of 72° C. for 5 minutes and a holding cycle of 4° C. The PCR products were loaded onto a 1.6% agarose gel to verify the fragment size.

One positive clone was selected and plasmid mini-prep DNA was prepared from 5 ml cultures using QIAprep Spin Miniprep kit (Qiagen).

Plasmid DNA (150-200 ng) in the pEAK12d vector was subjected to DNA sequencing with the sequencing primers pEAK12 FP and pEAK12 RP as described above. Plasmid DNA (150-200 ng) in the pDEST12.2 vector was subjected to DNA sequencing with the sequencing primers 21M13 FP and M13Rev RP as described above.

Sequence confirmed clones were designated as pEAK12d_INSP206SV3-6HIS and pDEST12.2_INSP206SV3-6HIS.

### 4.4 DNA Maxipreps

Maxi-prep DNA was prepared from a 500 ml culture of the sequence verified clone (pEAK12d_INSP206SV3-6HIS) using a Qiagen mega plasmid prep kit (cat no. 12183). Plasmid DNA was resuspended at a minimum concentration of 1 μg/μl in sterile water (or 10 mM Tris-HCl pH 8.5) and stored at −20° C.

Endotoxin-free maxi-prep DNA was prepared from a 500 ml culture of a sequence verified clone pDEST12.2_INSP206SV3-6HIS using the EndoFree Plasmid Mega kit (Qiagen cat no. 12381) according to the manufacturer's instructions. Purified plasmid DNA was resuspended in endotoxin free TE buffer at a final concentration of at least 3 μg/μl and stored at −20° C.

TABLE 5

| | |
|---|---|
| Primer | Sequence (5′-3′) |

| | |
|---|---|
| INSP206-F1 | ATGGGTGACCTGTGGTTATT (SEQ ID NO: |
| | 47) |
| INSP206-R2 | TTTAGCGACCTTCGTATTGC |
| | (SEQ ID NO: 48) |
| INSP206-F1 nest | GGTGACCTGTGGTTATTC (SEQ ID NO: |
| | 49) |
| INSP206-R1 nest | GTAACACAGCACTTCCCAGTA (SEQ ID NO: |
| | 50) |
| INSP206 attB FP | GCAGGCTTCGCCACCATGGGTGACCTGTGGTTAT |
| | TC (SEQ ID NO: 51) |
| INSP206SV3 attB | GTGATGGTGATGGTGACTTAACAACCTGCTGAAC |
| RP | (SEQ ID NO: 52) |
| ATTB PCR FP | GGGGACAAGTTTGTACAAAAAAGCAGGCTTC |
| | GCCACC (SEQ ID NO: 53) |
| ATTB PCR RP | GGGGACCACTTTGTACAAGAAAGCTGGGTTTCAA |
| | TGGTGATGGTGATGGTG (SEQ ID NO: 54) |
| pEAK12FP | AGCCTCAGACAGTGGTTCAA (SEQ ID NO: |
| | 55) |
| pEAK12RP | GATGGAGGTGGACGTGTCAG (SEQ ID NO: |
| | 56) |
| 21M13 FP | CGACGTTGTAAAACGACGGCCAGT (SEQ ID |
| | NO: 57) |
| M13REV RP | CAGGAAACAGCTATGAC (SEQ ID NO: 58) |
| T7 | TAATACGACTCACTATAGG (SEQ ID NO: |
| | 59) |
| T3 | ATTAACCCTCACTAAAGG (SEQ ID NO: 60) |

Underlined sequence = Kozak sequence
Bold = Stop codon
Italic sequence = His tag

### Example 5

### Cloning of INSP208SV1

### 5.1 Gene Specific Cloning Primers for PCR

PCR primers were designed for amplifying the predicted coding sequence of the virtual cDNA using Primer Designer Software (Scientific & Educational Software, PO Box 72045, Durham, N.C. 27722-2045, USA). Primers were selected which had high selectivity for the target sequence (INSP208).

5.2 Preparation of Human cDNA Templates

First strand cDNA was prepared from a variety of human tissue total RNA samples (Clontech, Stratagene, Ambion, Biochain Institute and in-house preparations) using SuperScript II or SuperScript III RNase H⁻ Reverse Transcriptase (Invitrogen) according to the manufacturer's protocol.

For SuperScript II: Oligo (dT)₁₅ primer (1 μl at 500 μg/ml) (Promega), 2 μg human total RNA, 1 μl 10 mM dNTP mix (10 mM each of dATP, dGTP, dCTP and dTTP at neutral pH) and sterile distilled water to a final volume of 12 μl were combined in a 1.5 ml Eppendorf tube, heated to 65° C. for 5 min and chilled on ice. The contents were collected by brief centrifugation and 4 μl of 5× First-Strand Buffer, 2 μl 0.1 M DTT, and 1 μl RnaseOUT™ Recombinant Ribonuclease Inhibitor (40 units/μl, Invitrogen) were added. The contents of the tube were mixed gently and incubated at 42° C. for 2 min, then 1 μl (200 units) of SuperScript II™ enzyme was added and mixed gently by pipetting. The mixture was incubated at 42° C. for 50 min and then inactivated by heating at 70° C. for 15 min. To remove RNA complementary to the cDNA, 1 μl (2 units) of E. coli RNase H (Invitrogen) was added and the reaction mixture incubated at 37° C. for 20 min.

For SuperScript III: 1 μl Oligo(dT)₂₀ primer (50 μM, Invitrogen), 2 μg human total RNA, 1 μl 10 mM dNTP mix (101n M each of dATP, dGTP, dCTP and dTTP at neutral pH) and sterile distilled water to a final volume of 10 μl were combined in a 1.5 ml Eppendorf tube, heated to 65° C. for 5 min and then chilled on ice. For each RT reaction a cDNA synthesis mix was prepared as follows: 2 μl 10×RT buffer, 4 μl 25 mM MgCl₂, 2 μl 0.1M DTT, 1 μl RNaseOUT™ (40 U/μl) and 1 μl SuperScript III™ RT enzyme were combined in a separate tube and then 10 μl of this mix added to the tube containing the RNA/primer mixture. The contents of the tube were mixed gently, collected by brief centrifugation, and incubated at 50° C. for 50 min. The reaction was terminated by incubating at 80° C. for 5 min and the reaction mixture then chilled on ice and collected by brief centrifugation. To remove RNA complementary to the cDNA, 1 μl (2 units) of E. coli RNase H (Invitrogen) was added and the reaction mixture incubated at 37° C. for 20 min.

The final 21 μl reaction mix was diluted by adding 179 μl sterile water to give a total volume of 200 μl. This represented approximately 20 ng/μl of each individual cDNA template.

5.3 PCR Amplification of INSP208 from Human cDNA Templates

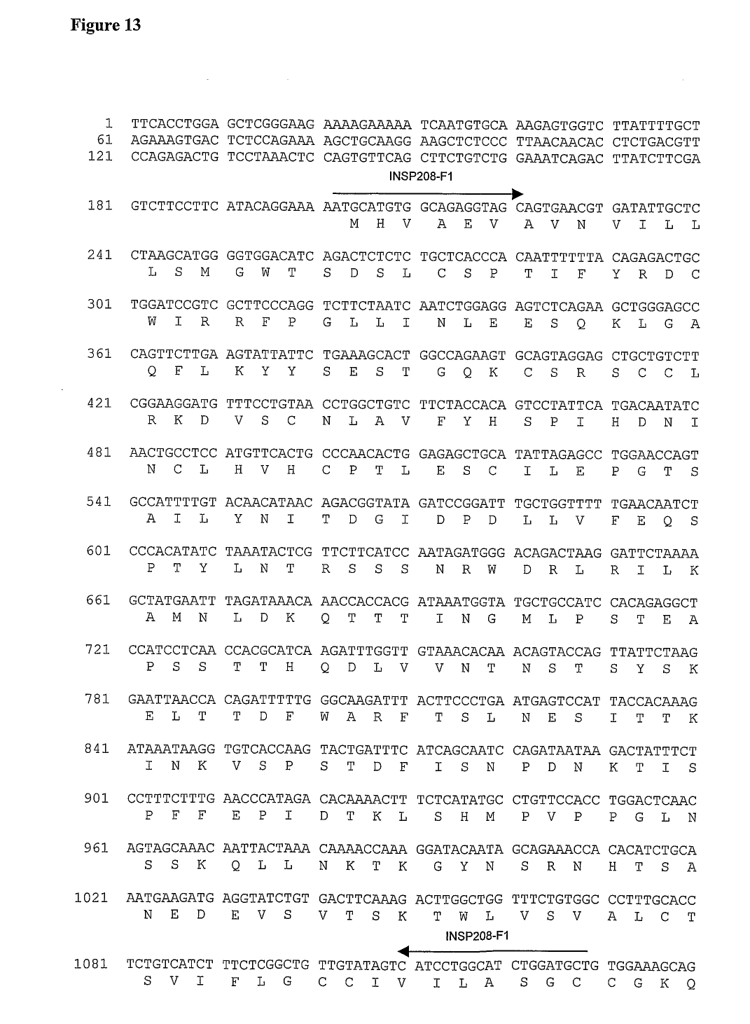

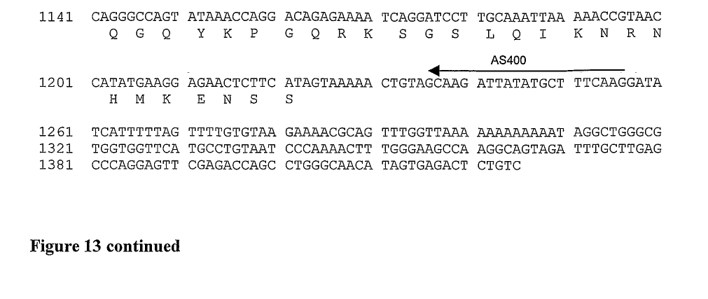

Gene-specific cloning primers (INSP208-F1 and INSP206-F2, Table 6 and FIG. 13) were designed to amplify a cDNA fragment of 927 bp containing the extracellular and transmembrane domain of the INSP208 cds. The primer pair was used to screen human cDNA templates and libraries derived from lung and pancreas as well as the universal reference RNA. PCR reactions were performed in a final volume of 50 μl containing 1× Taq High fidelity buffer, 200 μM dNTPs, 2 mM MgSO4, 0.2 μM of each cloning primer, 5 μl of 10× enhancer solution, 0.2 μl of Taq High fidelity, and 1 μl of each cDNA template. Cycling was performed using an MJ Research DNA Engine, programmed as follows: 94° C., 2 min; 40 cycles of 94° C., 30 sec, 52° C., 30 sec, and 68° C., 2 min; followed by 1 cycle at 68° C. for 8 min and a holding cycle at 4° C. PCR products (40 μl) were visualized on a 0.8% agarose gel in 1×TAE buffer (Invitrogen).

Products of approximately the expected molecular weight (927 bp) were purified from the gel using the MinElute Gel extraction kit (Qiagen), eluted in 10 μl of nuclease free water and subcloned directly.

### 5.4 Subcloning of PCR Products

The PCR products were subcloned into the topoisomerase I modified cloning vector (pCR4-TOPO) using the TA cloning kit purchased from the Invitrogen Corporation using the conditions specified by the manufacturer. Briefly, 4 μl of gel purified PCR product was incubated for 15 min at room temperature with 1 μl of TOPO vector and 1 μl salt solution. The reaction mixture was then transformed into E. coli strain TOP10 (Invitrogen) as follows: a 50 μl aliquot of One Shot TOP10 cells was thawed on ice and 2 μl of TOPO reaction was added. The mixture was incubated for 15 min on ice and then heat shocked by incubation at 42° C. for exactly 30 s. Samples were returned to ice and 250 μl of warm (room temperature) SOC media was added. Samples were incubated with shaking (220 rpm) for 1 h at 37° C. The transformation mixture was then plated on L-broth (LB) plates containing ampicillin (100 μg/ml) and incubated overnight at 37° C.

### 5.5 Colony PCR

Colonies were inoculated into 50 μl sterile water using a sterile toothpick. A 10 μl aliquot of the inoculum was then subjected to PCR in a total reaction volume of 20 μl containing 1× AmpliTaq™ buffer, 200 μM dNTPs, 20 pmoles of T7 primer, 20 pmoles of T3 primer, 1 unit of AmpliTaq™ (Applied Biosystems) using an MJ Research DNA Engine. The cycling conditions were as follows: 94° C., 2 min; 30 cycles of 94° C., 30 sec, 47° C., 30 sec and 72° C. for 1 min. Samples were maintained at 4° C. (holding cycle) before further analysis.

PCR products were analyzed on 1% agarose gels in 1×TAE buffer. Colonies which gave PCR products of approximately the expected molecular weight (927 bp+187 bp due to the multiple cloning site (MCS) were grown up overnight at 37° C. in 5 ml L-Broth (LB) containing ampicillin (100 μg/ml), with shaking at 220 rpm.

### 5.6 Plasmid DNA Preparation and Sequencing

Colonies which gave the expected band size by colony PCR were inoculated into 5 ml L-Broth (LB) containing ampicillin (100 μg/ml) and grown overnight at 37° C. with shaking at 220 rpm. Miniprep plasmid DNA was prepared from the 5 ml culture using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Plasmid DNA (200-500 ng) was subjected to DNA sequencing with the T7 and T3 primers using the BigDye Terminator system (Applied Biosystems cat. no. 4390246) according to the manufacturer's instructions. The primer sequences are shown in Table 6. Sequencing reactions were purified using Dye-Ex columns (Qiagen) or Montage SEQ 96 cleanup plates (Millipore cat. no. LSKS09624) then analyzed on an Applied Biosystems 3700 sequencer.

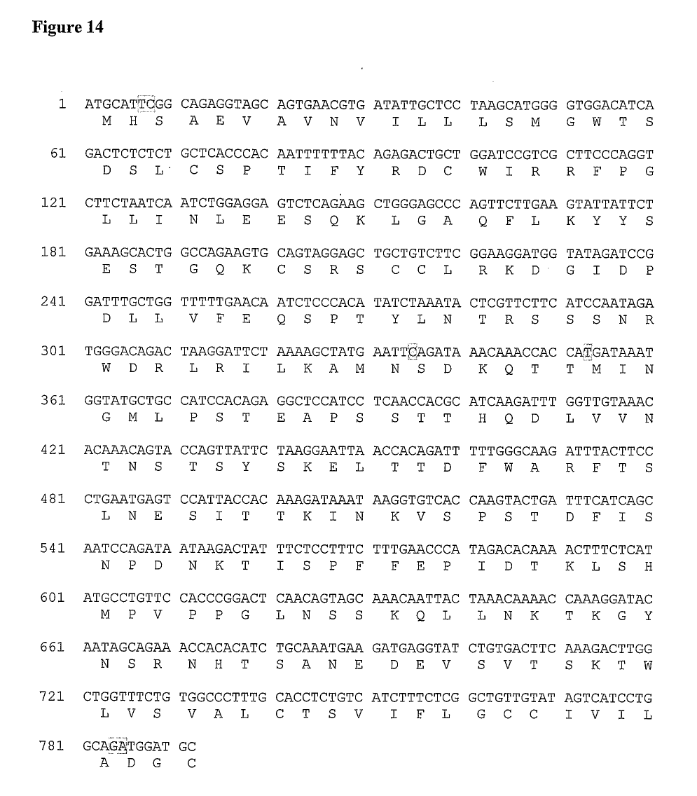

PCR products corresponding to the predicted INSP208 sequence were identified in clones analysed from S117 (cDNA derived from the universal reference RNA sample). The sequence was identical to the original prediction except that it contained a 135 bp in frame deletion (exon 2) and so appears to be a splice variant of INSP208 (INSP208SV). The cloned sequence also contained 6 point mutations G7T, T8C, T335C, C352T, T784G and C785A which would give rise to 4 amino acid substitutions corresponding to V3S, L112S, T118M and S262D in the original prediction. The mutations G7T, T8C, T784G and C785A are found in the sequence encoded by the INSP208F1 and INSP208R1 primer sequences respectively. Therefore it cannot be ruled out that these errors result from contaminants in the primer synthesis. L112S and T118M most probably represent PCR induced mutations. It is unlikely that any of the mutations represent polymorphisms as no ambiguity was detected at these positions when the sequence was blasted against the EMBL and Celera databases. These mutations were also absent in a full length INSP208 cDNA clone isolated subsequently (see example 8). The sequence of the cloned INSP208SV cDNA fragment is shown in FIG. 14. The plasmid containing the cloned PCR product is pCR4-TOPO-INSP208SV-S117-7.

### Example 6

### Construction of Mammalian Cell Expression Vectors for INSP208Sv

Plasmid pCR4-TOPO-INSP208SV-S117-7 was used as PCR template to generate pEAK12d and pDEST12.2 expression clones containing the ORF for the EC domain of INSP208SV (amino acids 1-240) with a 3′ sequence encoding a C-terminal 6HIS tag, using the Gateway™ cloning methodology (Invitrogen). The cDNA insert in plasmid 17642 contains 3 mutations (L112S, T118M and V3S) that needed to be corrected. The V3S mutation was corrected by introducing the change into the forward primer used to generate Gateway cloning compatible cDNA. Site directed mutagenesis was carried out to correct the other two mutations after the INSP208SV entry clone was created.

### 6.1 Generation of Gateway Compatible INSP208SV ORF Fused to an in Frame 6HIS Tag Sequence.

Plasmid pCR4-TOPO-INSP208SV-S117-7 was used as PCR template to generate the full-length ORF containing a C-terminal 6HIS tag and a stop codon. The first stage of this Gateway cloning process involved a two step PCR reaction which generates the full-length ORF of INSP208SV flanked at the 5′ end by an attB1 recombination site and Kozak sequence, and flanked at the 3′ end by a sequence encoding an in-frame 6 histidine (6HIS) tag, a stop codon and the attB2 recombination site (Gateway compatible cDNA). The first PCR reaction PCR1, (in a final volume of 50 μl) contains respectively: 1 μl (25 ng) of plasmid (17642), 4.0 μl dNTPs (10 mM), 5 μl of 10× Pfx polymerase buffer, 1.5 μl MgSO4 (50 mM), 1.0 μl each of gene specific primer (to give a final concentration of 100 pico-moles) (INSP208SV (S3V) attB FP and INSP208SV attB RP), and 0.5 μl Platinum Pfx DNA polymerase (Invitrogen). The PCR reaction was performed using an initial denaturing step of 95° C. for 2 min, followed by 30 cycles of 94° C. for 30 s; 60° C. for 30 s and 68° C. for 1 min; and a final extension cycle of 68° C. for 5 minutes and a holding cycle of 4° C. The amplification product was directly purified using the Perfectprep Gel cleanup kit (Eppendorf) and recovered in 50 μl sterile water according to the manufacturer's instructions. A 2 μl aliquot was visualized on 0.8% agarose gel in 1×TAE buffer in order to verify that the product was of the expected molecular weight (720 bp+30 bp=750 bp)

The second PCR reaction (in a final volume of 50 μl) contained 1 μl of diluted purified PCR1 product (to a final concentration of 10 ng), 4.0 μl dNTPs (10 mM), 5 μl of 10× Pfx polymerase buffer, 1.5 μl MgSO₄ (50 mM), 1.0 μl of each Gateway conversion primer (to give a final concentration of 100 picomoles) (ATTB PCR FP [unique] and ATTB PCR RP [unique]) and 0.5 μl of Platinum Pfx DNA polymerase. The conditions for the 2nd PCR reaction were: 95° C. for 2 min, followed by 30 cycles of 94° C. for 30 s; 60° C. for 30 s and 68° C. for 1 min; and a final extension cycle of 68° C. for 5 minutes and a holding cycle of 4° C. The PCR gel purified using the Perfectprep Gel cleanup kit (Eppendorf) and recovered in 50 μl sterile water according to the manufacturer's instructions. A 2 μl aliquot was visualized on 0.8% agarose gel in 1×TAE buffer in order to verify that the product was of the expected molecular weight (750 bp+58 bp=808 bp)

6.2 Sub Cloning of Gateway Compatible INSP208SV-6HIS ORF into Gateway Entry Vector pDONR221

The second stage of the Gateway cloning process involved sub cloning of the Gateway modified PCR product into the Gateway entry vector pDONR221 (Invitrogen) as follows: 5 μl of gel extracted product from PCR2 was incubated with 1.5 μl pDONR221 vector (0.1 μg/μl), 2 μl BP buffer and 1.5 μl of BP clonase enzyme mix (Invitrogen) in a final volume of 10 μl at RT for 1 h. The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 45 s. Samples were returned to ice and 250 μl of warm SOC media (room temperature) was added. Samples were incubated with shaking (250 rpm) for 1 h at 37° C. The transformation mixture was then plated on L-broth (LB) plates containing kanamycin (50 μg/ml) and incubated overnight at 37° C.

Five numbers of transformants were picked and patched on LB agar plates containing kanamycin (50 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μL dNTPs (10 mM), 2.5 μl of Taq polymerase buffer, 0.5 μl of screening primers (to give a final concentration of 100 picomoles) (21M13 FP and ATTB1 PCR RP [unique]) and 0.5 μl of Taq DNA polymerase.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 0.8% agarose gel to verify the fragment size.

One positive clone was selected and plasmid mini-prep DNA was prepared from 5 ml cultures using QIAprep Spin Miniprep kit (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 6. Sequencing reactions were analyzed on CEQ 2000 XL DNA analysis system (Beckman Coulter P/N 608450). After sequence confirmation of the insert, pDONR221-INSP208SV [L112S, T118M]-6HIS was used as a template for site-directed mutagenesis.

### 6.3 Site Directed Mutagenesis of INSP208SV-6HIS

The INSP208SV sequence cloned by PCR differed from the predicted INSP208SV, resulting in the two amino acid changes, L112S and T118M. These mutations were PCR induced as they were not detected in genomic DNA. In order to create a pDONR221 clone containing the correct INSP208SV sequence, the pDONR21_INSP208SV [L112S, T118M]-6HIS plasmid was used as a template for site-directed mutagenesis.

### 6.3.1 Gene Specific Cloning Primers for Site-Directed Mutagenesis of INSP208SV

The PCR primers, INSP208SV (S112L, M118T) FP and INSP208SV (S112L, M118T) RP (Table 6), were designed such that the primers annealed to opposite strands of the INSP208SV sequence and each primer annealed to 15-25 bases on either side of the amino acid to be mutated. The PCR primers were designed according to the instructions given in the Instruction manual for QuickChange® II XL Site-Directed Mutagenesis Kit (Stratagene).

### 6.3.2 Site-Direct Mutagenesis of INSP208SV

Site-directed mutagenesis, was carried out using the QuikChange® II Site-Directed Mutagenesis Kit (Stratagene) according to the manufacturer's instructions. The reaction was performed in a final volume of 50 μl containing 1× reaction buffer, 10 ng plasmid template DNA (pDONR221_INSP208SV [L112S, T118M]-6HIS), 125 ng INSP208SV (S112L, M118T) FP and INSP208SV (S112L, M118T) RP, 1 μl dNTP mix, and 2.5 units PfuUltra HF DNA polymerase. Thermal cycling was performed using a MJ Research DNA Engine, programmed as follows: 95° C., 1 min; 18 cycles of 95° C., 30 sec, 55° C., 1 min, and 68° C., 3 min 30 sec; followed by a holding cycle at 4° C.

Dpn I digestion was used to digest the methylated or hemimethylated parental DNA template (plasmid pDONR221_INSP208SV [L112S, T118M]-6HIS in the sample reaction). 1 μl of Dpn I restriction enzyme (10 U/μl, Stratagene) was added to the products of the amplification reactions. The reactions were mixed gently and incubated at 37° C. for 1 hour. The reaction mixture was then transformed into XL1-Blue supercompetent cells (Stratagene) as follows. A 50 μl aliquot of XL1-Blue cells was thawed on ice and 1 μl of Dpn I-treated DNA was added. The mixture was incubated for 30 min on ice and then heat shocked by incubation at 42° C. for exactly 45 s. Samples were returned to ice for 2 min and 250 μl of pre-warmed (42° C.) NZY media was added. Samples were incubated with shaking (220 rpm) for 1 h at 37° C. The transformation mixture (250 μl on each of 2 plates) was plated on L-broth (LB) plates containing kanamycin (50 μg/ml). Plates were incubated overnight at 37° C.

### 6.3.3 Plasmid DNA Preparation and Sequencing

One transformant was selected and plasmid mini-prep DNA was prepared from 5 ml cultures using QIAprep Spin Miniprep ldt (Qiagen). Plasmid DNA (150-200 ng) was subjected to DNA sequencing with 21M13 and M13Rev primers using the CEQ Dye Terminator Cycle sequencing Quick Start Kit (Beckman Coulter P/N 608120) according to the manufacturer's instructions. The primer sequences are shown in Table 6. Sequencing reactions were analyzed on CEQ 2000 XL DNA analysis system (Beckman Coulter P/N 608450). Sequence analysis identified a clone which contained the expected INSP208SV insert sequence (pDONR221_INSP208SV-6HIS).

6.4 Sub Cloning of Gateway Compatible INSP208SV ORF into Expression Vectors pEAK12d and pDEST12.2

Plasmid DNA (2 μl or approx. 150 ng) of pDONR221_INSP208SV-6HIS was then used in a recombination reaction containing 1.5 μl of either pEAK12d vector or pDEST12.2 vector (0.1 μg/μl), 2 μl LR buffer and 1.5 μl of LR clonase (Invitrogen) in a final volume of 10 μl.

The reaction was stopped by addition of 1 μl proteinase K (2 μg/μl) and incubated at 37° C. for a further 10 min. An aliquot of this reaction (2 μl) was used to transform DH5α strain (Invitrogen) as follows: a 50 μl aliquot of DH5α cells was thawed on ice and 2 μl of reaction mixture added. The mixture was incubated for 30 min on ice and then heat shocked by incubation at 42° C. for exactly 45 s. Samples were returned to ice and 250 μl of warm SOC media (room temperature) was added. Samples were incubated with shaking (250 rpm) for 1 h at 37° C. The transformation mixture was then plated on L-broth (LB) plates containing Ampicillin (100 μg/ml) and incubated overnight at 37° C.

Five numbers of transformants were picked and patched on LB agar plates containing Ampicillin (100 μg/ml) and incubated overnight at 37° C. A scoop of the grown culture from the patched plate was resuspended in 50 μl of water and boiled for 5 minutes to lyse the cells. The cell lysate was centrifuged to remove the cell debris and the supernatant obtained was used as a template for colony PCR screening.

The PCR mixture (in a final volume of 25 μl) contained 10 μl of the centrifuged cell lysate, 2.0 μl dNTPs (10 mM), 2.5 μl of Taq polymerase buffer, 0.5 μl of screening primers (to give a final concentration of 100 picomoles and 0.5 μl of Taq DNA polymerase. pEAK12d clones were screened using the primers pEAK12 FP and pEAK12 RP and pDEST12.2 clones were screened using the primers 21M13FP and M13Rev RP.

The conditions for the screening PCR reaction were: 95° C. for 2 min, followed by 30 cycles of 94° C. for 30 s; 60° C. for 30 s and 72° C. for 1 min; and a final extension cycle of 72° C. for 5 minutes and a holding cycle of 4° C. The PCR products were loaded onto a 0.8% agarose gel to verify the fragment size.

One positive clone was selected and plasmid mini-prep DNA was prepared from 5 ml cultures using QIAprep Spin Miniprep kit (Qiagen).

Plasmid DNA (150-200 ng) in the pEAK12d vector was subjected to DNA sequencing with the sequencing primers pEAK12 FP and pEAK12 RP as described above. Plasmid DNA (150-200 ng) in the pDEST12.2 vector was subjected to DNA sequencing with the sequencing primers 21M13 FP and M13Rev RP as described above.

Sequence confirmed clones were designated as pEAK12d_INSP208SV-6HIS and pDEST12.2_INSP208SV-6HIS (plasmid ID: 17994).

### 6.5 DNA Maxipreps

Maxi-prep DNA was prepared from a 500 ml culture of the sequence verified clone (pEAK12d_INSP208SV-6HIS) using a Qiagen mega plasmid prep kit (cat no. 12183). Plasmid DNA was resuspended at a minimum concentration of 1 μg/μl in sterile water (or 10 mM Tris-HCl pH 8.5) and stored at −20° C.

Endotoxin-free maxi-prep DNA was prepared from a 500 ml culture of a sequence verified clone pDEST12.2_INSP208SV-6HIS using the EndoFree Plasmid Mega kit (Qiagen cat no. 12381) according to the manufacturer's instructions. Purified plasmid DNA was resuspended in endotoxin free TE buffer at a final concentration of at least 3 μg/μl and stored at −20° C.

### Example 7

### Cloning of INSP208

7.1 RT-PCR From Human Multi-Tissue mRNA
7.1.1 Preparation of a Human Multi-Tissue cDNA Template

A preparation of human RNA was prepared by mixing approximately 10 μg total RNA from each of the following sources: Brain (Clontech), Heart (Clontech), Kidney (Clontech), Liver (Clontech), Lung (Clontech), Placenta (Clontech), Skeletal Muscle (Clontech), Small Intestine (Clontech), Spleen (Clontech), Thymus (Clontech), Uterus (Clontech) Bone Marrow (Clontech) Thyroid (Clontech), Ovary (Ambion), Prostate (Ambion), Skin (Resgen), Pancreas (Clontech), Salivary gland (BD Biosciences), Adrenal gland (BD Biosciences), Breast (Ambion), Pituitary gland (BioChain Institut), Stomach (Ambion), Mammary gland (Clontech), Lymph Node (BioChain Institut), Adipose tissue (BioChain Institut), Bladder (BioChain Institut), Appendix (BioChain Institut), Artery (BioChain Institut), Throat (BioChain Institut), Universal Human Reference (Stratagene), Foetal Kidney (Stratagene), Foetal Brain (BioChain Institut), Foetal Spleen (BioChain Institut), Foetal Liver (BioChain Institut), Foetal Heart (BioChain Institut), Foetal Lung (BioChain Institut).

The resulting pool of total RNA was fractionated by chromatography on a pre-packed oligo-dT column (Stratagene) according to the protocol supplied by the manufacturer. Approximately 400 μg total RNA yielded 12.6 μg polyA+ mRNA which was aliquoted and stored frozen at −80 C.

7.1.2 Synthesis of Gene Specific cDNAs

The gene specific cDNA primer for INSP208, AS400, was pooled with gene specific cDNA primers for 9 other predictions, each at a final concentration of 1 pM. The pooled cDNA primer set was diluted 10 fold into 50 μl of a mixture containing 1×RT buffer, 500 μM each dNTPs, 10 U/μl RNAguard (Pharmacia) and 1 μg denatured polyA⁺ RNA (prepared as described above). cDNA synthesis was initiated by addition of 10 U Omniscript reverse transcriptase (Qiagen) and allowed to proceed for 1 h at 37° C. At the end of the reaction, 5 μl of the cDNA mix was used for PCR amplification as described below.

### 7.1.3 PCR Amplification for INSP208

Top strand (AS401) and bottom strand (AS402) PCR primers (see Table 6) were designed to span the entire predicted coding sequence of INSP208. EcoR1 restriction sites were added at the 5′ end of each primer since no internal sites for this enzyme were predicted. A reaction mixture was set up containing 1×PCR buffer, 0.2 mM each dNTP, 0.5 μM each PCR primer, 5 μl cDNA template, and the PCR reaction was initiated by addition of 5 U PfuTurbo (Stratagene). Cycling conditions were: 95° C., 3 min (1 cycle); 95° C., 30 sec; 50° C., 30 sec and 75° C., 70 sec (35 cycles); 75° C., 10 min (1 cycle). An aliquot of the PCR reaction was analysed by electrophoresis on 0.8% agarose gels and the remainder was purified using the Wizard PCR Cleanup System (Promega) as recommended by the manufacturer, prior to subcloning of the PCR products.

### 7.2 Subcloning PCR Products

An aliquot of the purified PCR reation was digested with EcoR1 (New England Biolabs) for 2 h at 37° C. using the enzyme buffer supplied by the manufacturer.

In parallel, an appropriate amount of the Bluescript BSK cloning vector (Stratagene) was digested with EcoR1 and dephosphorylated using calf intestinal alkaline phosphatase (Roche Diagnostics) according to the supplier's recommendations. The full length linearized and dephosphorylated cloning vector was separated on a 0.8% agarose gel, and excised and purified using the Wizard Cleanup System (Promega) according to the protocol provided by the manufacturer. The purified vector DNA and PCR products were mixed in a molar ratio of 1:3 and precipitated overnight at −20° C. The precipitated DNA was recovered by centrifugation, washed in 70% ethanol, dried under vacuum and ligated in a final volume of 10 μl using the Rapid Ligation Kit (Roche Diagnostics) according to the protocol supplied by the manufacturer. The ligation mixture was then used to transform E. coli strain JM101 as follows: 50 μl aliquots of competent JM101 cells were thawed on ice and 1 μl or 5 μl of the ligation mixture reaction was added. The cells was incubated for 40 min on ice and then heat shocked by incubation at 42° C. for exactly 2 min. Warm (room temperature) L-Broth (LB) (1 ml) was added and samples were incubated for a further 1 h at 37° C. with shaking. The transformation mixture was then plated on LB plates containing ampicillin (100 μg/ml), IPTG (0.1 μM) and X-gal (50 μg/ml) and incubated overnight at 37° C. Single white colonies were chosen for plasmid isolation.

### 7.3 Plasmid DNA Preparation, Restriction Digestion and Sequence Analysis.

Miniprep plasmid DNA was prepared from 5 ml cultures using a Biorobot 8000 robotic system (Qiagen) or Wizard Plus SV Minipreps kit (Promega cat. no. 1460) according to the manufacturer's instructions. Plasmid DNA was eluted in 80 μl of sterile water. The DNA concentration was measured using an Eppendorf BO photometer or Spectramax 190 photometer (Molecular Devices). Aliquots of miniprep plasmid DNA (100-200 ng) were digested with EcoR1 for 2 h at 37° C. and analysed by electrophoresis on 0.8% agarose gels. Plasmids with inserts of the appropriate size were selected for DNA sequence analysis. Inserts were sequenced in both directions by mixing 200-500 ng plasmid DNA with either the T7 or T3 sequencing primers (see Table 6), and processed using the BigDye Terminator system (Applied Biosystems cat. no. 4390246) according to the manufacturer's instructions. Sequencing reactions were purified using Dye-Ex columns (Qiagen) or Montage SEQ 96 cleanup plates (Millipore cat. no. LSKS09624) then analyzed on an Applied Biosystems 3700 sequencer.

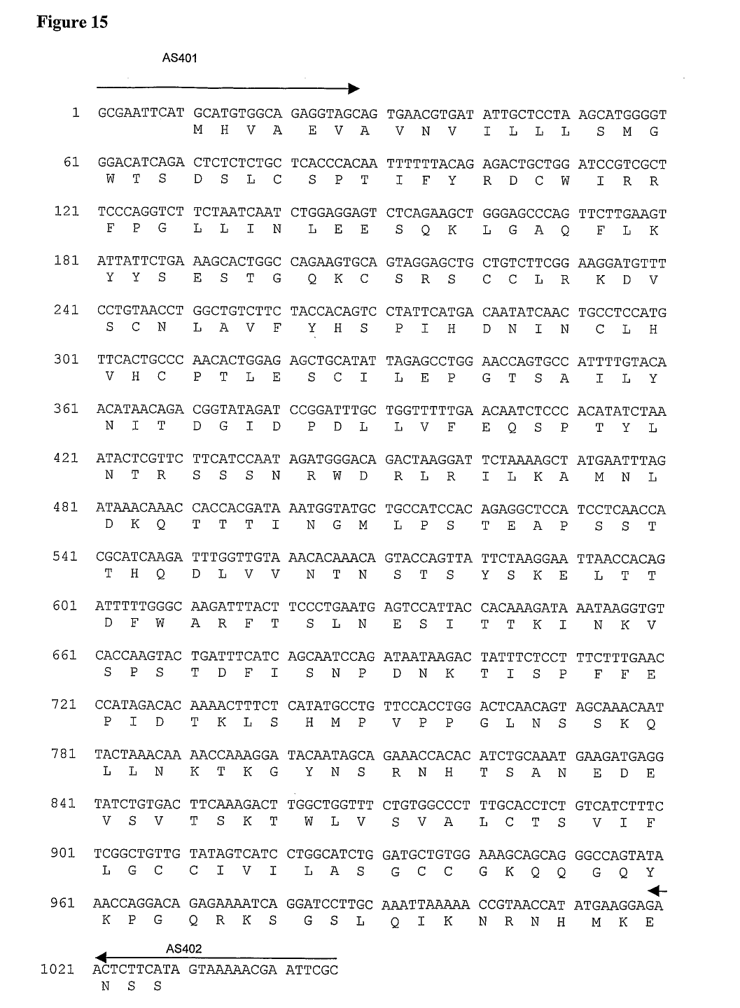

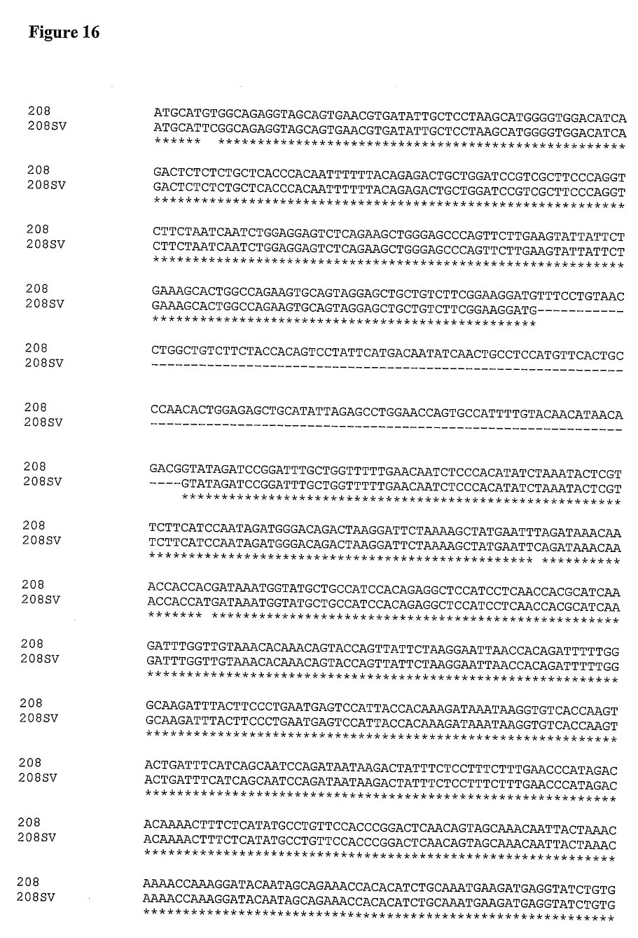

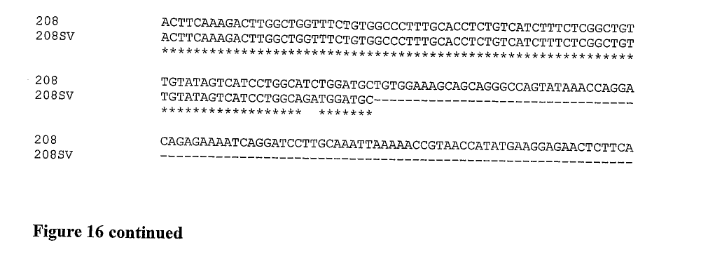

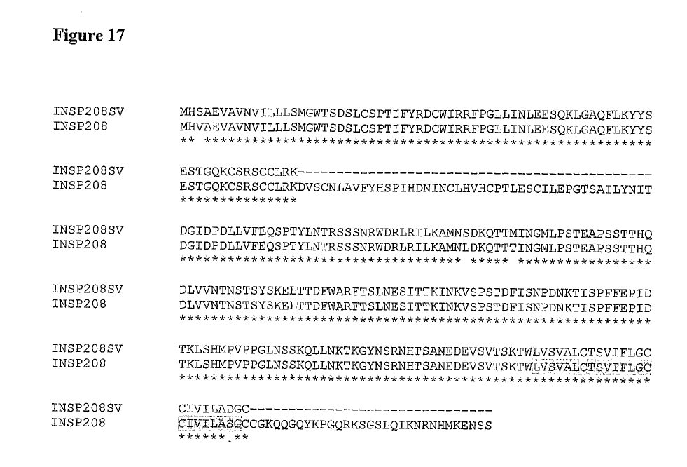

PCR products corresponding to the predicted INSP208 sequence were identified in several of the clones analysed. The sequence of the cloned cDNA fragment is shown in FIG. 15. The plasmid containing the sequence verified full length INSP208 PCR product is INSP208_pBSK.

TABLE 6

| | |
|---|---|
| Primer | Sequence (5′-3′) |

| | |
|---|---|
| INSP208-F1 | ATGCATGTGGCAGAGGTAGC (SEQ ID NO: |
| | 61) |
| INSP208-R2 | GCATCCAGATGCCAGGATGA (SEQ ID NO: |
| | 62) |
| INSP208SV | CTAAAAGCTATGAATTTAGATAAACAAACCACCACGATAAATGGTATGCTGC |
| (S112L, M118T) FP | (SEQ ID NO: 63) |
| AS400 | CTTGAAAGCATATAATCTTGC (SEQ ID NO: 64) |
| AS401 | GCGAATTCATGCATGTGGCAGAGGTAGC (SEQ ID NO: 65) |
| AS402 | GCGAATTCGTTTTTACTATGAAGAGTTC (SEQ ID NO: 66) |
| INSP208SV | GCAGCATACCATTTATCGTGGTGGTTTGTTTATCTAAATTCATAGCTTTTAG |
| (S112L, M118T) RP | (SEQ ID NO: 67) |
| INSP208SV [S3V] | GCAGGCTTCGCCACCATGCATGTGGCAGAGGTAGCAG |
| attB FP | (SEQ ID NO: 68) |
| INSP208SV attB | GTGATGGTGATGGTGCCAAGTCTTTGAAGTC (SEQ ID NO: 69) |
| RP | |
| ATTB PCR FP | GGGGACAAGTTTGTACAAAAAAGCAGGCTTCGCCACC (SEQ ID NO: 53) |
| ATTB PCR RP | GGGGACCACTTTGTACAAGAAAGCTGGGTTTCAATGGTGATGGTGATGGTG |
| | (SEQ ID NO: 54) |
| pEAK12FP | AGCCTCAGACAGTGGTTCAA (SEQ ID NO: 55) |
| pEAK12RP | GATGGAGGTGGACGTGTCAG (SEQ ID NO: 56) |
| 21M13 FP | CGACGTTGTAAAACGACGGCCAGT (SEQ ID NO: 57) |
| M13REV RP | CAGGAAACAGCTATGAC (SEQ ID NO: 58) |
| T7 | TAATACGACTCACTATAGG (SEQ ID NO: 59) |
| T3 | ATTAACCCTCACTAAAGG (SEQ ID NO: 60) |

Underlined sequence = Kozak sequence
Bold = Stop codon
Italic sequence = His tag

### Example 8

### Analysis of INSP206 Gene Expression

### 8.1 Analysis of INSP206 Gene Expression Levels by Real Time PCR (Taqman)

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, and dTTP, and DEPC-treated water in a volume of 10 μl. The mixture was incubated at 65° C. for 5 min then chilled on ice for 1 min. The following 10 μl cDNA synthesis mix was prepared in a separate tube: 2 μl 10×RT buffer, 4 μl 125 mM MgCl₂, 2 μl 0.1 M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700). PCR primers for human INSP206 and the housekeeping control gene glyceraldehyde 3-phosphate dehydrogenase (GAPDH) were designed using the Primer Express software (PE Biosystems). The INSP206 forward primer was designed in exon 1 and the reverse primer was designed in exon 2.

The sequences of the primers are shown in Table 7. The specificity and the optimal primer concentration to use for the TaqMan analysis were determined by testing the INSP206 primers on a series of dilutions of plasmid pEAK12d/INSP206SV2-6HIS (plasmid ID. 17992), pEAK12d/INSP206-6HIS (plasmid ID. 17963) and pEAK12d/INSP206SV1-6HIS (plasmid ID. 17957). Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the INSP206 gene in each cDNA sample was expressed as a difference in Ct value, i.e. Delta (δ) Ct=Ct (GAPDH)−Ct (INSP206). Results for each sample were then expressed as a fold difference in the number of cycles required for detectable INSP206 gene expression relative to that for GAPDH, according to the formula Fold Difference=2^{(-δCt)}. Finally, the expression level of the INSP206 gene in each cDNA sample was shown relative to the GAPDH gene expression level, where GAPDH expression level=100%, by dividing 100 by the Fold Difference for INSP206.

### 8.2 Results

This primer pair used in this study is specific for INSP206. INSP206 primers were tested on a panel of approx. 100 normal and diseased human tissue samples, primary cells and cell lines in addition to inflammatory bowel disease colon and ileum biopsies and psoriasis biopsies from an IL18BP clinical trial. Results are shown in tables 8-12. Using primers in exon 1 (F) and exon 2(R), INSP206 mRNA was detectable at low levels in testis (0.13% of GAPDH) and skin (0.12% of GAPDH) (Table 8). Very high expression was detected in two osteoarthritis (OA) synovium samples (13.5% and 2.2% of GAPDH respectively) (Table 10). Low expression was also detected in one normal dermal fibroblast sample; in a basophilic leukaemia cell line (KU812) (0.56% of GAPDH) as well as in granulocytes, PBMCs and the eosinophilic cell line, EOL-3 (0.28% of GAPDH) (Table 11). Low expression of INSP206 was also detected in most of the IBD samples tested (Table 12).

TABLE 7
TaqMan PCR primer sequences

| | |
|---|---|
| Primer | Sequence (5′-3′) |

| | |
|---|---|
| INSP206-98F | GCTCCTTGCTGGGAAATCTG (SEQ ID NO: 70) |
| INSP206-255R | TGTTCTCAACTTAACAACCTGCTGA (SEQ ID |
| | NO: 71) |
| HGAPDH-F | CCACCCATGGCAAATTCC (SEQ ID NO: 72) |
| HGAPDH-R | GATGGGATTTCCATTGATGACA (SEQ ID |
| | NO: 73) |
| Intron-hGAPDH- | CCTAGTCCCAGGGCTTTGATT (SEQ ID |
| F | NO: 74) |
| Intron-hGAPDH- | CTGTGCTCCCACTCCTGATTT (SEQ ID |
| R | NO: 75) |

TABLE 8
Expression of INSP206 in major human tissues as measured by Sybr green RT-
PCR.

| major tissues | Ct | Ct | delta | | |
|---|---|---|---|---|---|
| collection | hGAPDH | hINSP206 | ct | Fold difference | Relative to GAPDH (=100) |
| S76 Brain | 20.61 | 34.73 | −14.11 | 17724.74 | 0.01 |
| S77 Heart | 22.39 | 34.54 | −12.15 | 4542.14 | 0.02 |
| S78 Kidney | 21.30 | 32.59 | −11.29 | 2504.78 | 0.04 |
| S79 liver | 22.00 | 36.20 | −14.20 | 18803.57 | 0.01 |
| S80 Lung | 23.17 | 40.00 | −16.83 | 116713.10 | 0.00 |
| S81 Placenta | 21.63 | 34.90 | −13.28 | 9919.55 | 0.01 |
| S82 skeletal Muscle | 16.68 | 40.00 | −23.32 | 10507178.66 | 0.00 |
| S83 small intestine | 21.85 | 40.00 | −18.15 | 291286.94 | 0.00 |
| S84 Spleen | 21.08 | 34.37 | −13.29 | 9989.98 | 0.01 |
| S85 Thymus | 19.87 | 40.00 | −20.13 | 1146562.84 | 0.00 |
| S86 Uterus | 21.80 | 33.64 | −11.85 | 3682.62 | 0.03 |
| S89 Spinal cord | 19.90 | 33.83 | −13.93 | 15577.23 | 0.01 |
| S90 Cervix | 23.55 | 40.00 | −16.45 | 89804.62 | 0.00 |
| S91 colon | 21.85 | 40.00 | −18.15 | 291514.78 | 0.00 |
| S92 ovary | 23.87 | 40.00 | −16.13 | 71883.20 | 0.00 |
| S93 prostate | 20.99 | 40.00 | −19.01 | 528815.71 | 0.00 |
| S94 testis | 21.57 | 31.13 | −9.56 | 754.99 | 0.13 |
| S95 skin | 25.61 | 35.26 | −9.65 | 804.85 | 0.12 |
| S113 pancreas | 23.04 | 40.00 | −16.96 | 127149.26 | 0.00 |
| S119 Breast | 21.33 | 40.00 | −18.67 | 418167.23 | 0.00 |
| S120 Stomach | 23.57 | 39.30 | −15.73 | 54485.40 | 0.00 |
| S122 Eye | 22.71 | 38.67 | −15.96 | 63695.13 | 0.00 |
| S147 Bladder | 21.35 | 38.64 | −17.29 | 160689.10 | 0.00 |

TABLE 9
Expression of INSP206 in comparative human tissues as
measured by Sybr green RT-PCR.

| | Ct | Ct | delta | | |
|---|---|---|---|---|---|
| Comparative tissues | hGAPDH | hINSP206 | ct | Fold difference | Relative to GAPDH (=100) |
| S76 Brain | 21.11 | 38.19 | −17.08 | 138140.37 | 0.00 |
| S140 fetal Brain | 20.50 | 40.00 | −19.50 | 741080.89 | 0.00 |
| S77 Heart | 23.68 | 40.00 | −16.32 | 82059.88 | 0.00 |
| S143 fetal Heart | 21.54 | 40.00 | −18.46 | 361178.88 | 0.00 |
| S78 Kidney | 21.53 | 40.00 | −18.47 | 363796.21 | 0.00 |
| S121 fetal Kidney | 20.97 | 37.01 | −16.03 | 67130.28 | 0.00 |
| S130 Lupus total RNA | 23.14 | 40.00 | −16.86 | 119357.77 | 0.00 |
| Kidney | | | | | |
| S135 Kidney tumor | 21.00 | 40.00 | −19.00 | 524549.72 | 0.00 |
| S79 liver | 24.54 | 40.00 | −15.46 | 45230.07 | 0.00 |
| S142 fetal liver | 21.60 | 40.00 | −18.40 | 344820.38 | 0.00 |
| S127 Lupus total RNA liver | 24.57 | 40.00 | −15.43 | 44118.31 | 0.00 |
| S131 Liver Cirrhosis | 22.88 | 40.00 | −17.12 | 142808.62 | 0.00 |
| S136 Liver Tumor | 18.68 | 40.00 | −21.32 | 2610780.07 | 0.00 |
| S80 Lung | 24.91 | 40.00 | −15.09 | 34840.42 | 0.00 |
| S144 Fetal Lung | 19.30 | 40.00 | −20.70 | 1706886.86 | 0.00 |
| S128 Lupus total RNA Lung | 21.44 | 38.04 | −16.60 | 99042.63 | 0.00 |
| S132 Cirrhosis Lung | 18.20 | 40.00 | −21.80 | 3652086.49 | 0.00 |
| S137 Lung Tumor | 21.40 | 30.97 | −9.56 | 756.46 | 0.13 |
| S84 Spleen | 23.46 | 38.50 | −15.04 | 33583.57 | 0.00 |
| S141 Fetal Spleen | 21.01 | 40.00 | −18.99 | 521953.23 | 0.00 |
| S129 Lupus total RNA | 24.32 | 40.00 | −15.68 | 52663.89 | 0.00 |
| Spleen | | | | | |
| S133 Cirrhosis Spleen | 22.29 | 40.00 | −17.71 | 214256.06 | 0.00 |
| S117 Human Universal | 17.90 | 31.16 | −13.26 | 9788.95 | 0.01 |
| Reference | | | | | |

TABLE 10
Expression of INSP206 in human secretory and immune tissues as measured by
Sybr green RT-PCR.

| Secretory and immunology | Ct | Ct | delta | | |
|---|---|---|---|---|---|
| samples | hGAPDH | hINSP206 | ct | Fold difference | Relative to GAPDH (=100) |
| S87 Bone Marrow | 22.21 | 40.00 | −17.79 | 227040.63 | 0.00 |
| S88 Thyroid | 20.85 | 34.17 | −13.31 | 10186.92 | 0.01 |
| S115 Salivary Gland | 21.27 | 34.56 | −13.29 | 10027.03 | 0.01 |
| S116 Adrenal Gland | 19.76 | 40.00 | −20.24 | 1241081.56 | 0.00 |
| S123 Mammary gland | 23.66 | 33.67 | −10.01 | 1032.21 | 0.10 |
| S125 Pituitary Gland | 21.59 | 29.65 | −8.06 | 266.98 | 0.37 |
| S145 Lymph Node | 23.59 | 40.00 | −16.41 | 87069.03 | 0.00 |
| S146 Adipose | 19.47 | 40.00 | −20.53 | 1515608.28 | 0.00 |
| S148 Appendix | 21.78 | 40.00 | −18.22 | 306233.38 | 0.00 |
| S149 Blood vessel Artery | 22.00 | 40.00 | −18.00 | 262120.38 | 0.00 |
| S150 Throat | 20.03 | 37.64 | −17.62 | 201122.02 | 0.00 |
| S54 Stroma | 22.30 | 40.00 | −17.70 | 212651.20 | 0.00 |
| S153 cells HDMEC | 22.28 | 40.00 | −17.72 | 215825.60 | 0.00 |
| S157 cells HDMEC stimuled | 22.80 | 40.00 | −17.20 | 150660.74 | 0.00 |
| S155 cell HAoEC | 21.48 | 40.00 | −18.52 | 375997.10 | 0.00 |
| S158 cell HAoEC stimuled | 20.50 | 40.00 | −19.50 | 741610.17 | 0.00 |
| S11 RA2 | 21.43 | 29.08 | −7.65 | 200.63 | 0.50 |
| S12 RA3 | 21.63 | 32.34 | −10.71 | 1674.81 | 0.06 |
| S13 OA1 | 26.05 | 28.94 | −2.89 | 7.40 | 13.52 |
| S19 OA4 | 23.76 | 29.29 | −5.53 | 46.26 | 2.16 |

TABLE 11
Expression of INSP206 in human primary cells and cell lines as measured by Sybr
green RT-PCR.

| Primary cells and cell | Ct | Ct | delta | | |
|---|---|---|---|---|---|
| lines 1 | hGAPDH | hINSP206 | ct | Fold difference | Relative to GAPDH (=100) |
| S1 AG1518 fibroblast | 20.54 | 40.00 | −19.46 | 719990.08 | 0.00 |
| S2 Howard Ab | 21.55 | 32.39 | −10.84 | 1835.17 | 0.05 |
| S3 Clark N | 21.72 | 40.00 | −18.28 | 317930.52 | 0.00 |
| S4 NF1 | 19.55 | 28.04 | −8.49 | 359.63 | 0.28 |
| S5 NF2 | 22.12 | 33.96 | −11.83 | 3648.38 | 0.03 |
| S6 SScN2 | 22.34 | 35.44 | −13.10 | 8800.66 | 0.01 |
| S7 SSCA2 | 18.76 | 34.03 | −15.27 | 39490.09 | 0.00 |
| S15 LN1 | 20.85 | 33.56 | −12.71 | 6704.99 | 0.01 |
| S16 Lab1 | 19.57 | 40.00 | −20.43 | 1414861.10 | 0.00 |
| S17 LN14 | 18.62 | 29.87 | −11.25 | 2438.66 | 0.04 |
| S18 LA13 | 19.10 | 32.28 | −13.18 | 9307.14 | 0.01 |
| S9 NHDF2 | 23.05 | 37.60 | −14.55 | 23909.16 | 0.00 |
| S10 NHDF3 | 21.61 | 34.54 | −12.92 | 7763.50 | 0.01 |
| S55 JEHC | 20.49 | 40.00 | −19.51 | 748660.44 | 0.00 |
| S56 HT 1080 | 19.10 | 40.00 | −20.90 | 1961060.35 | 0.00 |
| S57 MRC-5 | 20.29 | 33.50 | −13.21 | 9470.11 | 0.01 |
| S152 Cells Mob | 24.16 | 40.00 | −15.84 | 58504.76 | 0.00 |
| S155 Cells Mob stimuled | 22.32 | 40.00 | −17.68 | 209769.30 | 0.00 |
| S156 Cells Mob stimuled | 18.90 | 35.14 | −16.23 | 77064.00 | 0.00 |
| S20 K1 Keratinocytes | 23.20 | 40.00 | −16.80 | 114454.05 | 0.00 |
| Skin | | | | | |
| S21 K2 Keratinocytes | 23.12 | 38.19 | −15.07 | 34514.60 | 0.00 |
| Skin | | | | | |
| S30 THP-1 mono/mac | 20.48 | 34.25 | −13.77 | 13975.16 | 0.01 |
| S35 KU812 basophil | 21.93 | 29.40 | −7.47 | 177.26 | 0.56 |
| S37 KU812/PMA | 21.33 | 33.36 | −12.02 | 4158.73 | 0.02 |
| S43 Jurkat | 20.32 | 33.47 | −13.15 | 9068.97 | 0.01 |
| S58 PBMC1 | 23.52 | 40.00 | −16.48 | 91320.81 | 0.00 |
| S59 Granulocytes 1 | 24.47 | 33.34 | −8.88 | 470.57 | 0.21 |
| S61 PBMC2.2 | 21.79 | 31.65 | −9.86 | 926.25 | 0.11 |
| S97 SK-N-AS | 19.44 | 31.92 | −12.48 | 5709.19 | 0.02 |
| S98 TE671 subclone 2 | 21.14 | 33.85 | −12.72 | 6729.71 | 0.01 |
| S99 KELLY | 19.36 | 40.00 | −20.64 | 1629374.72 | 0.00 |
| S100 U-373 MG | 21.60 | 40.00 | −18.40 | 345841.99 | 0.00 |
| S101 U-87 MG | 22.35 | 40.00 | −17.65 | 204986.16 | 0.00 |
| S102 T98G | 17.93 | 40.00 | −22.07 | 4415243.27 | 0.00 |
| S103 BE(2)-C | 19.78 | 35.36 | −15.58 | 48935.03 | 0.00 |
| S104 CCF-STGG1 | 22.97 | 40.00 | −17.03 | 133961.09 | 0.00 |
| S105 TE671 | 26.70 | 40.00 | −13.30 | 10097.55 | 0.01 |
| S106 A172 | 21.35 | 35.22 | −13.87 | 15010.54 | 0.01 |
| S107 132N1 | 22.40 | 40.00 | −17.60 | 198921.47 | 0.00 |
| S108 SK-PN-DW | 21.18 | 35.16 | −13.98 | 16200.38 | 0.01 |
| S109 SK-N-SH | 27.22 | 40.00 | −12.78 | 7021.36 | 0.01 |
| S38 MOLT-4 | 24.43 | 40.00 | −15.57 | 48794.74 | 0.00 |
| S41 EOL-3 | 24.45 | 32.92 | −8.47 | 354.33 | 0.28 |
| S44 EOL-3 + IL2 | 25.24 | 35.51 | −10.26 | 1230.30 | 0.08 |

TABLE 12
Expression of INSP206 in human colon and ileum biopsies

| IBD | Ct | Ct | delta | | |
|---|---|---|---|---|---|
| plate | hGAPDH | hINSP206 | ct | Fold difference | Relative to GAPDH (=100) |
| N1 | 25.08 | 30.93 | −5.85 | 57.76 | 1.73 |
| N2 | 24.53 | 32.11 | −7.58 | 191.05 | 0.52 |
| N3 | 25.02 | 32.41 | −7.39 | 167.88 | 0.60 |
| N4 | 23.21 | 30.83 | −7.62 | 196.38 | 0.51 |
| N5 | 24.67 | 37.06 | −12.40 | 5391.06 | 0.02 |
| N9 | 22.49 | 40.00 | −17.51 | 187107.13 | 0.00 |
| N10 | 24.50 | 31.37 | −6.88 | 117.39 | 0.85 |
| CD1 | 24.26 | 32.59 | −8.32 | 320.42 | 0.31 |
| CD2 | 24.40 | 31.97 | −7.57 | 190.33 | 0.53 |
| CD3 | 22.57 | 29.80 | −7.22 | 149.21 | 0.67 |
| CD4 | 25.05 | 29.48 | −4.44 | 21.68 | 4.61 |
| CD5 | 25.85 | 40.00 | −14.15 | 18171.27 | 0.01 |
| CD6 | 23.60 | 30.98 | −7.38 | 166.36 | 0.60 |
| CD8 | 23.65 | 29.93 | −6.28 | 77.77 | 1.29 |
| CD9 | 22.51 | 30.07 | −7.56 | 188.13 | 0.53 |
| CD10 | 24.07 | 30.62 | −6.55 | 93.75 | 1.07 |
| CD13 | 23.08 | 29.08 | −6.00 | 64.07 | 1.56 |
| CD16 | 25.40 | 31.17 | −5.78 | 54.80 | 1.82 |
| CD17 | 24.99 | 31.68 | −6.69 | 103.45 | 0.97 |
| CD18 | 26.43 | 40.00 | −13.57 | 12175.94 | 0.01 |
| CD19 | 22.56 | 30.92 | −8.36 | 328.10 | 0.30 |
| CD20 | 23.31 | 31.41 | −8.10 | 274.42 | 0.36 |
| CD22 | 24.43 | 31.69 | −7.26 | 153.58 | 0.65 |
| N11 | 24.36 | 31.56 | −7.20 | 147.20 | 0.68 |
| N14 | 24.49 | 40.00 | −15.51 | 46508.41 | 0.00 |
| N26 | 25.45 | 32.36 | −6.90 | 119.78 | 0.83 |
| N27 | 25.48 | 31.27 | −5.79 | 55.30 | 1.81 |
| N29 | 26.24 | 31.98 | −5.74 | 53.47 | 1.87 |
| N30 | 23.37 | 29.11 | −5.73 | 53.21 | 1.88 |
| CD4 bis | 24.77 | 33.39 | −8.62 | 392.30 | 0.25 |
| CD6 bis | 24.50 | 39.57 | −15.08 | 34546.43 | 0.00 |
| CD23 | 24.97 | 31.16 | −6.19 | 73.09 | 1.37 |
| CD24 | 23.61 | 29.82 | −6.22 | 74.54 | 1.34 |
| CD25 | 23.67 | 32.43 | −8.76 | 432.46 | 0.23 |
| CD26 | 24.19 | 37.27 | −13.08 | 8655.01 | 0.01 |
| CD27 | 26.94 | 40.00 | −13.06 | 8543.61 | 0.01 |
| CD28 | 26.25 | 40.00 | −13.75 | 13757.91 | 0.01 |
| UC11 | 25.66 | 33.07 | −7.41 | 170.48 | 0.59 |
| UC12 | 23.61 | 34.87 | −11.26 | 2447.79 | 0.04 |
| UC13 | 25.93 | 34.21 | −8.28 | 310.87 | 0.32 |
| UC14 | 24.25 | 30.74 | −6.50 | 90.43 | 1.11 |
| UC16 | 25.63 | 32.18 | −6.54 | 93.26 | 1.07 |
| UC18 | 26.15 | 39.86 | −13.71 | 13411.76 | 0.01 |
| UC19 | 25.86 | 40.00 | −14.14 | 18000.48 | 0.01 |

### Example 9

### Analysis of INSP208 Gene Expression

### 9.1 Analysis of INSP208 Gene Expression Levels by Real Time PCR (Taqman)

Total RNA from each sample was reverse transcribed using the Superscript III First-Strand Synthesis System for RT-PCR (Invitrogen, Cat. No. 18080-051) in a final reaction volume of 20 μl. 2 μg of total RNA was combined with 50 ng random hexamer primers, 10 mM each of dATP, dGTP, dCTP, and dTTP, and DEPC-treated water in a volume of 10 μl. The mixture was incubated at 65° C. for 5 min then chilled on ice for 1 min. The following 10 μl cDNA synthesis mix was prepared in a separate tube: 2 μl 10×RT buffer, 4 μl 25 mM MgCl₂, 2 μl 0.1 M DTT, 1 μl RnaseOUT™ (40 units/μl), and 1 μl SuperScript™ III RT enzyme (200 units/μl). The cDNA synthesis mix was added to the RNA/primer mixture, mixed gently and incubated at 25° C. for 10 min then at 50° C. for 50 min. The RT enzyme was then inactivated by incubating at 85° C. for 5 min. The mixture was chilled on ice and then 1 μl of E. coli Rnase H (2 units/μl) was added and the mixture incubated at 37° C. for 20 min. The mixture was chilled on ice and then diluted 1/250 with sterile water. Dilutions of the reverse transcriptase reaction were then subjected to real time PCR analysis on a TaqMan instrument (PE Biosystems 7700). PCR primers for human INSP208 and the housekeeping control gene glyceraldehyde 3-phosphate dehydrogenase (GAPDH) were designed using the Primer Express software (PE Biosystems). The INSP208 forward primer was designed in exon 2 and the reverse primer was designed in exon 3.

The sequences of the primers are shown in Table 13. The specificity and the optimal primer concentration to use for the TaqMan analysis were determined by testing the INSP208 primers on a series of dilutions of plasmid 18451 (pEAK12D INSP208EC-6HIS). Potential genomic DNA contamination of the cDNA was excluded by performing PCR reactions using primers specific for GAPDH intronic sequence. The absence of non-specific amplification was controlled by analyzing the PCR products on 4% agarose gels to ensure a single band of the expected molecular weight was produced.

SYBR Green Real-Time PCR reactions were carried out in a reaction volume of 50 μl containing 25 μl SYBR Green PCR master mix (PE Biosystems) (to which 0.5 units AmpErase Uracil N-Glycosylase (UNG, PE Biosystems) had previously been added), 300 nM of each amplification primer, and 5 μl of RT-PCR product. Cycling was performed using the ABI PRISM 7700 (TaqMan) Detection System programmed as follows: 1 cycle of 50° C. for 2 min; 1 cycle of 95° C. for 10 min; 40 cycles of 95° C. for 15 sec, 60° C. for 1 min. Each reaction was carried out in duplicate and the results averaged.

The primer-specific regions of the reverse-transcribed cDNA samples were thus amplified and their cycle threshold (Ct) values determined. The Ct value for each cDNA sample was normalized to that of the housekeeping gene GAPDH as follows. The difference in expression level between the GAPDH gene and the INSP208 gene in each cDNA sample was expressed as a difference in Ct value, i.e. Delta (δ) Ct=Ct (GAPDH)−Ct (INSP208). Results for each sample were then expressed as a fold difference in the number of cycles required for detectable INSP208 gene expression relative to that for GAPDH, according to the formula Fold Difference=2^{(-δCt)}. Finally, the expression level of the INSP208 gene in each cDNA sample was shown relative to the GAPDH gene expression level, where GAPDH expression level=100%, by dividing 100 by the Fold Difference for INSP208.

### 9.2 Results

This primer pair used in this study is specific for INSP208. INSP208 primers were tested on a panel of approx. 100 normal and diseased human tissue samples, primary cells and cell lines in addition to inflammatory bowel disease colon and ileum biopsies and psoriasis biopsies from an IL 18BP clinical trial. Results are shown in tables 14-18. Using primers in exon 2 (F) and exon 3 (R), INSP208 mRNA was detectable at low levels in stomach (0.26% of GAPDH), pancreas (0.23% of GAPDH), testis (0.13% of GAPDH) and ovary (0.12% of GAPDH) (Table 14); in tonsil (0.26% of GAPDH) (Table 16); in normal primary keratinocytes (0.11% of GAPDH) (Table 17) as well as in a number of psoriasis skin biopsy samples (Table 18).

TABLE 13
TaqMan PCR primer sequences

| | |
|---|---|
| Primer | Sequence (5′-3′) |

| | |
|---|---|
| INSP208-337F2 | TGCATATTAGAGCCTGGAACCA (SEQ |
| | ID NO: 76) |
| INSP208-412R1 | CCATCTATTGGATGAAGAACGAGTATT |
| | (SEQ ID NO: 77) |
| hGAPDH-F | CCACCCATGGCAAATTCC (SEQ ID |
| | NO: 72) |
| hGAPDH-R | GATGOGATTTCCATTGATGACA (SEQ |
| | ID NO: 73) |
| Intron-hGAPDH-F | CCTAGTCCCAGGGCTTTGATT (SEQ ID |
| | NO: 74) |
| Intron-hGAPDH-R | CTGTGCTCCCACTCCTGATTT (SEQ ID |
| | NO: 75) |

TABLE 14
Expression of INSP208 in major human tissues as measured by
Sybr green RT-PCR.

| major tissues | Ct | Ct | delta | | |
|---|---|---|---|---|---|
| collection | hGAPDH | hINSP208 | ct | Fold difference | Relative to GAPDH (=100) |
| S76 Brain | 20.66 | 33.42 | −12.75 | 6912.22 | 0.01 |
| S77 Heart | 22.34 | 37.19 | −14.84 | 29352.22 | 0.00 |
| S78 Kidney | 21.26 | 33.33 | −12.07 | 4285.41 | 0.02 |
| S79 liver | 22.56 | 34.07 | −11.51 | 2916.16 | 0.03 |
| S80 Lung | 22.84 | 34.91 | −12.07 | 4291.26 | 0.02 |
| S81 Placenta | 23.08 | 34.04 | −10.96 | 1991.61 | 0.05 |
| S82 skeletal Muscle | 16.81 | 36.03 | −19.21 | 607252.29 | 0.00 |
| S83 small intestine | 21.38 | 35.60 | −14.21 | 18992.21 | 0.01 |
| S84 Spleen | 23.83 | 36.34 | −12.51 | 5846.77 | 0.02 |
| S85 Thymus | 20.40 | 33.59 | −13.19 | 9338.56 | 0.01 |
| S86 Uterus | 21.39 | 32.58 | −11.19 | 2332.90 | 0.04 |
| S89 Spinal cord | 21.54 | 33.60 | −12.06 | 4269.09 | 0.02 |
| S90 Cervix | 23.51 | 35.49 | −11.97 | 4025.39 | 0.02 |
| S91 colon | 21.87 | 34.63 | −12.76 | 6951.34 | 0.01 |
| S92 ovary | 23.51 | 33.22 | −9.71 | 839.55 | 0.12 |
| S93 prostate | 20.80 | 33.54 | −12.74 | 6841.92 | 0.01 |
| S94 testis | 21.64 | 31.18 | −9.54 | 744.82 | 0.13 |
| S95 skin | 26.01 | 36.13 | −10.12 | 1113.73 | 0.09 |
| S113 pancreas | 24.48 | 33.48 | −9.00 | 512.08 | 0.20 |
| S119 Breast | 21.92 | 35.05 | −13.13 | 8985.44 | 0.01 |
| S120 Stomach | 23.59 | 32.16 | −8.58 | 381.45 | 0.26 |
| S122 Eye | 21.92 | 32.69 | −10.77 | 1751.22 | 0.06 |
| S147 Bladder | 20.96 | 32.76 | −11.80 | 3554.44 | 0.03 |

TABLE 15
Expression of INSP208 in comparative human tissues as measured by Sybr green
RT-PCR.

| | Ct | Ct | delta | | |
|---|---|---|---|---|---|
| Comparative tissues | hGAPDH | hINSP208 | ct | Fold difference | Relative to GAPDH (=100) |
| S76 Brain | 21.50 | 35.05 | −13.56 | 12056.34 | 0.01 |
| S140 fetal Brain | 20.38 | 31.47 | −11.10 | 2190.23 | 0.05 |
| S77 Heart | 23.73 | 33.95 | −10.21 | 1187.37 | 0.08 |
| S143 fetal Heart | 21.32 | 34.01 | −12.69 | 6626.55 | 0.02 |
| S78 Kidney | 21.89 | 33.53 | −11.64 | 3198.96 | 0.03 |
| S121 fetal Kidney | 20.61 | 32.67 | −12.06 | 4280.46 | 0.02 |
| S130 Lupus total RNA | 22.71 | 34.32 | −11.61 | 3120.75 | 0.03 |
| Kidney | | | | | |
| S135 Kidney tumor | 21.49 | 35.12 | −13.63 | 12658.35 | 0.01 |
| S79 liver | 23.44 | 33.66 | −10.22 | 1195.37 | 0.08 |
| S142 fetal liver | 21.28 | 33.92 | −12.64 | 6378.16 | 0.02 |
| S131 Liver Cirrhosis | 22.96 | 34.32 | −11.36 | 2626.09 | 0.04 |
| S136 Liver Tumor | 21.14 | 34.36 | −13.22 | 9548.30 | 0.01 |
| S80 Lung | 24.39 | 35.05 | −10.67 | 1626.08 | 0.06 |
| S144 Fetal Lung | 19.22 | 33.73 | −14.51 | 23358.45 | 0.00 |
| S128 Lupus total RNA Lung | 21.12 | 33.34 | −12.22 | 4760.87 | 0.02 |
| S132 Cirrhosis Lung | 18.62 | 32.00 | −13.38 | 10696.67 | 0.01 |
| S137 Lung Tumor | 23.72 | 34.80 | −11.09 | 2173.86 | 0.05 |
| S84 Spleen | 23.21 | 34.06 | −10.84 | 1838.24 | 0.05 |
| S141 Fetal Spleen | 22.41 | 34.77 | −12.36 | 5258.55 | 0.02 |
| S129 Lupus total RNA | 23.47 | 36.19 | −12.72 | 6736.89 | 0.01 |
| Spleen | | | | | |
| S133 Cirrhosis Spleen | 23.03 | 33.81 | −10.78 | 1761.30 | 0.06 |
| S117 Human Universal | 17.60 | 33.54 | −15.94 | 62857.53 | 0.00 |
| Reference | | | | | |

TABLE 16
Expression of INSP208 in human secretory and immune tissues as measured by
Sybr green RT-PCR.

| Secretory and immunology | Ct | Ct | delta | | |
|---|---|---|---|---|---|
| samples | hGAPDH | hINSP208 | ct | Fold difference | Relative to GAPDH (=100) |
| S87 Bone Marrow | 22.45 | 32.65 | −10.20 | 1178.16 | 0.08 |
| S88 Thyroid | 21.28 | 34.92 | −13.64 | 12781.73 | 0.01 |
| S115 Salivary Gland | 21.77 | 34.00 | −12.23 | 4803.97 | 0.02 |
| S116 Adrenal Gland | 22.03 | 34.30 | −12.27 | 4948.32 | 0.02 |
| S123 Mammary gland | 24.35 | 35.18 | −10.83 | 1826.56 | 0.05 |
| S125 Pituitary Gland | 23.02 | 34.64 | −11.62 | 3142.86 | 0.03 |
| S145 Lymph Node | 23.50 | 34.03 | −10.53 | 1480.65 | 0.07 |
| S146 Adipose | 21.39 | 34.14 | −12.76 | 6918.66 | 0.01 |
| S148 Appendix | 22.94 | 34.66 | −11.72 | 3371.14 | 0.03 |
| S149 Blood vessel Artery | 21.83 | 34.21 | −12.38 | 5324.46 | 0.02 |
| S150 Throat | 21.47 | 34.71 | −13.24 | 9692.65 | 0.01 |
| S75 Tonsil | 24.53 | 33.11 | −8.59 | 384.81 | 0.26 |
| S54 Stroma | 21.84 | 33.12 | −11.29 | 2500.30 | 0.04 |
| S153 cells HDMEC | 22.25 | 35.32 | −13.07 | 8570.97 | 0.01 |
| S157 cells HDMEC stimuled | 22.63 | 34.72 | −12.09 | 4369.59 | 0.02 |
| S155 cell HAoEC | 21.24 | 33.89 | −12.66 | 6458.24 | 0.02 |
| S158 cell HAoEC stimuled | 20.61 | 34.50 | −13.89 | 15204.17 | 0.01 |
| S11 RA2 | 21.72 | 34.21 | −12.49 | 5753.52 | 0.02 |
| S12 RA3 | 21.08 | 34.86 | −13.78 | 14103.70 | 0.01 |
| S19 OA4 | 22.30 | 36.35 | −14.05 | 16940.80 | 0.01 |

TABLE 17
Expression of INSP208 in human primary cells and cell lines as measured by Sybr
green RT-PCR.

| Primary cells and cell | Ct | Ct | delta | | |
|---|---|---|---|---|---|
| lines 1 | hGAPDH | hINSP208 | ct | Fold difference | Relative to GAPDH (=100) |
| S1 AG1518 fibroblast | 20.66 | 33.52 | −12.86 | 7439.73 | 0.01 |
| S2 Howard Ab | 20.55 | 34.60 | −14.05 | 16940.05 | 0.01 |
| S3 Clark N | 22.10 | 34.97 | −12.87 | 7511.72 | 0.01 |
| S4 NF1 | 20.80 | 33.42 | −12.62 | 6313.89 | 0.02 |
| S5 NF2 | 22.00 | 35.54 | −13.54 | 11888.32 | 0.01 |
| S6 SScN2 | 20.43 | 35.48 | −15.06 | 34047.52 | 0.00 |
| S7 SSCA2 | 20.15 | 31.87 | −11.72 | 3383.63 | 0.03 |
| S15 LN1 | 18.82 | 33.62 | −14.80 | 28600.63 | 0.00 |
| S16 Lab1 | 17.98 | 34.25 | −16.27 | 78976.56 | 0.00 |
| S17 LN14 | 18.78 | 32.91 | −14.13 | 17879.13 | 0.01 |
| S18 LA13 | 18.00 | 34.33 | −16.33 | 82233.81 | 0.00 |
| S9 NHDF2 | 21.18 | 33.13 | −11.94 | 3936.63 | 0.03 |
| S10 NHDF3 | 21.22 | 36.61 | −15.39 | 42926.43 | 0.00 |
| S55 JEHC | 19.64 | 35.37 | −15.73 | 54286.49 | 0.00 |
| S56 HT 1080 | 18.37 | 35.12 | −16.75 | 110237.61 | 0.00 |
| S57 MRC-5 | 18.92 | 34.03 | −15.12 | 35544.08 | 0.00 |
| S152 Cells Mob | 20.68 | 31.98 | −11.30 | 2522.88 | 0.04 |
| S155 Cells Mob stimuled | 20.42 | 34.50 | −14.08 | 17373.06 | 0.01 |
| S156 Cells Mob stimuled | 21.94 | 33.41 | −13.85 | 14802.80 | 0.01 |
| S20 K1 Keratinocytes | 22.35 | 33.90 | −11.55 | 2999.20 | 0.03 |
| Skin | | | | | |
| S21 K2 Keratinocytes | 24.05 | 33.83 | −9.78 | 879.19 | 0.11 |
| Skin | | | | | |
| S160 JAR Trophoblast | 20.60 | 37.06 | −16.46 | 90158.79 | 0.00 |
| S30 THP-1 mono/mac | 20.81 | 34.04 | −13.23 | 9612.42 | 0.01 |
| S35 KU812 basophil | 19.67 | 33.96 | −14.29 | 19983.51 | 0.01 |
| S37 KU812/PMA | 20.89 | 33.42 | −12.53 | 5910.41 | 0.02 |
| S43 Jurkat | 19.37 | 34.28 | −14.91 | 30717.62 | 0.00 |
| S58 PBMC1 | 23.65 | 34.68 | −11.03 | 2089.50 | 0.05 |
| S59 Granulocytes 1 | 24.71 | 33.54 | −8.84 | 457.02 | 0.22 |
| S61 PBMC2.2 | 22.57 | 33.90 | −11.33 | 2566.56 | 0.04 |
| S97 SK-N-AS | 22.31 | 33.80 | −11.50 | 2889.54 | 0.03 |
| S98 TE671 subclone 2 | 20.78 | 34.08 | −13.30 | 10070.69 | 0.01 |
| S99 KELLY | 19.79 | 34.59 | −14.80 | 28602.37 | 0.00 |
| S100 U-373 MG | 21.70 | 33.81 | −12.11 | 4420.78 | 0.02 |
| S101 U-87 MG | 20.06 | 33.91 | −13.85 | 14816.70 | 0.01 |
| S102 T98G | 21.30 | 33.21 | −11.90 | 3832.80 | 0.03 |
| S103 BE(2)-C | 21.34 | 34.30 | −12.96 | 7961.98 | 0.01 |
| S104 CCF-STGG1 | 21.75 | 34.23 | −12.49 | 5735.72 | 0.02 |
| S106 A172 | 21.47 | 32.79 | −11.32 | 2558.77 | 0.04 |
| S107 132N1 | 22.93 | 33.92 | −10.99 | 2033.38 | 0.05 |
| S108 SK-PN-DW | 20.91 | 33.86 | −12.95 | 7913.12 | 0.01 |
| S38 MOLT-4 | 23.21 | 33.54 | −10.33 | 1289.16 | 0.08 |
| S41 EOL-3 | 23.88 | 34.88 | −10.99 | 2037.66 | 0.05 |
| S44 EOL-3 + IL2 | 24.08 | 34.11 | −10.03 | 1048.17 | 0.10 |

TABLE 18
Expression of INSP208 in human psoriasis skin biopsy samples

| | | | | | Relative to |
|---|---|---|---|---|---|
| Psoriasis | Ct | Ct | delta | | GAPDH |
| Patient ID | hGAPDH | hINSP208 | ct | Fold difference | (=100) |
| 11 | 20.94 | 32.82 | −11.88 | 3769.09 | 0.03 |
| 16 | 23.45 | 32.81 | −9.36 | 657.11 | 0.15 |
| 28 | 23.42 | 32.22 | −8.80 | 445.72 | 0.22 |
| 36 | 24.20 | 32.30 | −8.10 | 274.37 | 0.36 |
| 39 | 22.98 | 34.47 | −11.49 | 2876.30 | 0.03 |
| 59 | 24.66 | 33.37 | −8.71 | 418.77 | 0.24 |
| 60 | 21.79 | 33.15 | −11.36 | 2628.46 | 0.04 |
| 63 | 23.64 | 31.29 | −7.65 | 200.85 | 0.50 |
| 64 | 21.54 | 34.06 | −12.52 | 5873.48 | 0.02 |
| 66 | 21.67 | 31.86 | −10.19 | 1168.14 | 0.09 |
| 68 | 23.13 | 33.81 | −10.68 | 1640.59 | 0.06 |
| 69 | 23.99 | 32.84 | −8.85 | 461.44 | 0.22 |
| 70 | 24.38 | 34.30 | −9.92 | 968.76 | 0.10 |
| 71 | 26.53 | 33.46 | −6.93 | 121.94 | 0.82 |
| 72 | 23.88 | 33.38 | −9.50 | 724.08 | 0.14 |
| 73 | 23.25 | 34.53 | −11.28 | 2486.67 | 0.04 |
| 74 | 22.83 | 33.07 | −10.24 | 1209.34 | 0.08 |
| 75 | 18.37 | 33.10 | −14.73 | 27175.14 | 0.00 |
| 77 | 22.80 | 33.15 | −10.35 | 1305.15 | 0.08 |
| 78 | 22.92 | 32.22 | −9.30 | 630.35 | 0.16 |
| 79 | 20.55 | 33.15 | −12.60 | 6208.38 | 0.02 |
| 80 | 23.27 | 33.68 | −10.41 | 1360.57 | 0.07 |
| 81 | 23.42 | 31.71 | −8.29 | 313.00 | 0.32 |
| 82 | 24.02 | 31.98 | −7.96 | 249.00 | 0.40 |
| 83 | 23.20 | 32.71 | −9.51 | 729.11 | 0.14 |
| 84 | 22.52 | 32.41 | −9.89 | 948.83 | 0.11 |
| 85 | 23.43 | 33.31 | −9.88 | 942.27 | 0.11 |
| 86 | 23.52 | 32.64 | −9.12 | 556.41 | 0.18 |
| 88 | 23.62 | 33.83 | −10.21 | 1184.45 | 0.08 |
| 91 | 25.21 | 33.01 | −7.80 | 222.86 | 0.45 |
| 96 | 24.16 | 33.20 | −9.04 | 526.39 | 0.19 |
| 97 | 25.53 | 32.59 | −7.06 | 133.44 | 0.75 |
| 98 | 24.31 | 32.69 | −8.38 | 333.14 | 0.30 |
| 99 | 21.17 | 32.74 | −11.57 | 3040.30 | 0.03 |
| 101 | 21.87 | 34.27 | −12.40 | 5404.70 | 0.02 |
| 102 | 24.34 | 32.88 | −8.54 | 372.22 | 0.27 |
| 103 | 21.37 | 33.02 | −11.65 | 3213.66 | 0.03 |

1-53. (canceled)

54. An isolated polypeptide that:
(a) comprises SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40;
(b) consists of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40;
(c) has greater than 80% sequence identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40; or
(d) a fusion protein comprising a heterologous sequence fused to a polypeptide comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40.

55. The polypeptide according to claim 54, wherein said polypeptide has greater than 85% sequence identity.

56. The polypeptide according to claim 54, wherein said polypeptide has greater than 90% sequence identity.

57. The polypeptide according to claim 54, wherein said polypeptide has greater than 95% sequence identity.

58. The polypeptide according to claim 54, wherein said polypeptide has greater than 98% sequence identity.

59. A purified nucleic acid molecule that:
(a) encodes SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40;
(b) comprises SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 or SEQ ID NO:39;
(c) consists of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, SEQ ID NO:37 or SEQ ID NO:39;
(d) hybridizes under high stringency conditions with a nucleic acid molecule encoding encodes SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40; or
(e) is a vector comprising a nucleic acid molecule that encodes SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40.

60. A host cell transformed with a nucleic acid according to claim 59.

61. A ligand which binds specifically to a polypeptide according to claim 54.

62. The ligand according to claim 61, wherein said ligand is an antibody.

63. A method of diagnosing a disease in a patient comprising assessing the level of expression of a natural gene or a polypeptide in tissue from said patient and comparing said level of expression or activity to a control level, wherein a level that is different to said control level is indicative of disease and wherein said:
a) natural gene encodes a gene product that encodes a polypeptide comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40; or
b) polypeptide comprises SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40.

64. The method according to claim 63, wherein said method is performed in vitro.

65. The method according to claim 63, wherein said method assesses the level of polypeptide expression and comprises the steps of: (a) contacting a ligand that specifically binds to said polypeptide with a biological sample under conditions suitable for the formation of a ligand-polypeptide complex; and (b) detecting said complex.

66. The method according to claim 63, wherein said method assesses the level natural gene expression and comprises:
a) contacting a sample of tissue from the patient with a nucleic acid probe that hybridizes with said gene product under stringent conditions that allow the formation of a hybrid complex between said natural gene and the probe;
b) contacting a control sample with said probe under the same conditions used in step a); and
c) detecting the presence of hybrid complexes in said samples; wherein detection of levels of the hybrid complex in the patient sample that differ from levels of the hybrid complex in the control sample is indicative of disease.

67. The method according to claim 63, wherein said method assesses the level of a natural gene expression and comprises:
a) contacting a sample of nucleic acid from tissue of the patient with a nucleic acid primer under stringent conditions that allow the formation of a hybrid complex between a primer and said natural gene product;
b) contacting a control sample with said primer under the same conditions used in step a);
c) amplifying the sampled nucleic acid; and
d) detecting the level of amplified nucleic acid from both patient and control samples; wherein detection of levels of the amplified nucleic acid in the patient sample that differ significantly from levels of the amplified nucleic acid in the control sample is indicative of disease.

68. The method according to claim 63, wherein said method assesses the level of a natural gene and comprises:
a) obtaining a tissue sample from a patient being tested for disease;
b) isolating a nucleic acid molecule encoding a polypeptide comprising SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:38 or SEQ ID NO:40; and
c) diagnosing the patient for disease by detecting the presence of a mutation which is associated with disease in the nucleic acid molecule as an indication of the disease.

69. The method according to claim 68, further comprising amplifying the nucleic acid molecule to form an amplified product and detecting the presence or absence of a mutation in the amplified product.

70. The method according to claim 68, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

71. The method according to claim 69, wherein the presence or absence of the mutation in the patient is detected by contacting said nucleic acid molecule with a nucleic acid probe that hybridizes to said nucleic acid molecule under stringent conditions to form a hybrid double-stranded molecule, the hybrid double-stranded molecule having an unhybridized portion of the nucleic acid probe strand at any portion corresponding to a mutation associated with disease; and detecting the presence or absence of an unhybridized portion of the probe strand as an indication of the presence or absence of a disease-associated mutation.

72. The method according to claim 63, wherein said disease is a fertility disorder, neoplasm, melanoma, lung, colorectal, breast, pancreas, head and neck or other solid tumor, leukemia, non-Hodgkin lymphoma, leukopenia, thrombocytopenia, angiogenesis disorder, Kaposi's' sarcoma, autoimmune/inflammatory disorders, allergy, inflammatory bowel disease, arthritis, osteoarthritis, psoriasis, respiratory tract inflammation, asthma, organ transplant rejection, hypertension, edema, angina, atherosclerosis, thrombosis, sepsis, shock, reperfusion injury, ischemia, Alzheimer's disease, brain injury, amyotrophic lateral sclerosis, pain, diabetes mellitus, osteoporosis, obesity, AIDS viral infection, bacterial infection, fungal infection or parasitic infection.

73. A pharmaceutical composition comprising a polypeptide according to claim 54 and a carrier.

74. A method of treating a disease in a patient comprising administering to the patient a polypeptide according to claim 54 or a nucleic acid encoding said polypeptide in an amount effective to treat said disease in said patient.

75. A transgenic or knockout non-human animal that has been transformed to express higher, lower or absent levels of a polypeptide according to claim 54.

76. A method for screening for a compound effective to treat disease, by contacting a non-human transgenic animal according to claim 75 with a candidate compound and determining the effect of the compound on the disease of the animal.

77. A method of selecting biologically active compounds that bind to a Cys-rich polypeptide comprising:
i) contacting a candidate compound with recombinant host cells expressing an INSP206 or INSP208 polypeptide; and
ii) selecting compounds that bind said INSP206 or INSP208 polypeptide at the surface of said cells and/or that modulate the activity of the INSP206 or INSP208 polypeptide.
